SEQUENCE LISTING (24 sequences)

SEQ ID NO: 1 - DNA, 4679 bp, synthetic construct
  source 1..4679: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttccttgcgtcgacagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagaagcttagggtgagtctatgggacccttgatgttttctttccccttcttttctatggttaagttcatgtcataggaaggggataagtaacagggtacacatattgaccaaatcagggtaattttgcatttgtaattttaaaaaatgctttcttcttttaatatacttttttgtttatcttatttctaatactttccctaatctctttctttcaaggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctccaccggtcgccaccatgaagcgagcagctgcaaagcatctaatagaacgctactaccaccagttaactgagggctgtggaaatgaagcctgcacgaatgagttttgtgcttcctgtccaacttttcttcgtatggataataatgcagcagctattaaagccctcgagctttataagattaatgcaaaactctgtgatcctcatccctccaagaaaggagcaagctcagcttaccttgagaactcgaaaggtgcccccaacaactcctgctctgagataaaaatgaacaagaaaggcgctagaattgattttaaagatgtgacttacttaacagaagagaaggtatatgaaattcttgaattatgtagagaaagagaggattattcccctttaatccgtgttattggaagagttttttctagtgctgaggcattggtacagagcttccggaaagttaaacaacacaccaaggaagaactgaaatctcttcaagcaaaagatgaagacaaagatgaagatgaaaaggaaaaagctgcatgttctgctgctgctatggaagaagactcagaagcatcttcctcaaggataggtgatagctcacagggagacaacaatttgcaaaaattaggccctgatgatgtgtctgtggatattgatgccattagaagggtctacaccagattgctctctaatgaaaaaattgaaactgcctttctcaatgcacttgtatatttgtcacctaacgtggaatgtgacttgacgtatcacaatgtatactctcgagatcctaattatctgaatttgttcattatcgtaatggagaatagaaatctccacagtcctgaatatctggaaatggctttgccattattttgcaaagcgatgagcaagctaccccttgcagcccaaggaaaactgatcagactgtggtctaaatacaatgcagaccagattcggagaatgatggagacatttcagcaacttattacttataaagtcataagcaatgaatttaacagtcgaaatctagtgaatgatgatgatgccattgttgctgcttcgaagtgcttgaaaatggtttactatgcaaatgtagtgggaggggaagtggacacaaatcacaatgaagaagatgatgaagagcccatccctgagtccagcgagctgacacttcaggaacttttgggagaagaaagaagaaacaagaaaggtcctcgagtggaccccctggaaactgaacttggtgttaaaaccctggattgtcgaaaaccacttatcccttttgaagagtttattaatgaaccactgaatgaggttctagaaatggataaagattatacttttttcaaagtagaaacagagaacaaattctcttttatgacatgtccctttatattgaatgctgtcacaaagaatttgggattatattatgacaatagaattcgcatgtacagtgaacgaagaatcactgttctctacagcttagttcaaggacagcagttgaatccatatttgagactcaaagttagacgtgaccatatcatagatgatgcacttgtccggctagagatgatcgctatggaaaatcctgcagacttgaagaagcagttgtatgtggaatttgaaggagaacaaggagttgatgagggaggtgtttccaaagaattttttcagctggttgtggaggaaatcttcaatccagatattggtatgttcacatacgatgaatctacaaaattgttttggtttaatccatcttcttttgaaactgagggtcagtttactctgattggcatagtactgggtctggctatttacaataactgtatactggatgtacattttcccatggttgtctacaggaagctaatggggaaaaaaggaacttttcgtgacttgggagactctcacccagttctatatcagagtttaaaagatttattggagtatgaagggaatgtggaagatgacatgatgatcactttccagatatcacagacagatctttttggtaacccaatgatgtatgatctaaaggaaaatggtgataaaattccaattacaaatgaaaacaggaaggaatttgtcaatctttattctgactacattctcaataaatcagtagaaaaacagttcaaggcttttcggagaggttttcatatggtgaccaatgaatctcccttaaagtacttattcagaccagaagaaattgaattgcttatatgtggaagccggaatctagatttccaagcactagaagaaactacagaatatgacggtggctataccagggactctgttctgattagggagttctgggaaatcgttcattcatttacagatgaacagaaaagactcttcttgcagtttacaacgggcacagacagagcacctgtgggaggactaggaaaattaaagatgattatagccaaaaatggcccagacacagaaaggttacctacatctcatacttgctttaatgtgcttttacttccggaatactcaagcaaagaaaaacttaaagagagattgttgaaggccatcacgtatgccaaaggatttggcatgctctaagtttaaacgattcgaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtgaagtgggtaacctttatttcccttctttttctctttagctctgcttattccaggggtgtgtttaggagagatgcacacaagagtgaggttgctcataggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttagggaaacctatggtgaataggctgactgctgtgcaaaacaagaacctgagagaaatgaatgcttcttgcaacacaaagatgacaacccaaacctccccagattggtgagaccagaggttgatgtgtagtgcactgcttttcatgacaatgaagagacctttttgaaaaaatacttatatgaaattgccagaagacatccttacttttatgcccctgaactccttttctttgctaaaaggtataaagctgcttttacagaatgttgccaagctgctgataaagctgccggcgcgccatcgatgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 2 - DNA, 145 bp, synthetic construct
  source 1..145: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 3 - DNA, 463 bp, synthetic construct
  source 1..463: mol_type = other DNA
agggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 4 - DNA, 482 bp, synthetic construct
  source 1..482: mol_type = other DNA
agggtgagtctatgggacccttgatgttttctttccccttcttttctatggttaagttcatgtcataggaaggggataagtaacagggtacacatattgaccaaatcagggtaattttgcatttgtaattttaaaaaatgctttcttcttttaatatacttttttgtttatcttatttctaatactttccctaatctctttctttcaaggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctc

SEQ ID NO: 5 - DNA, 2559 bp, synthetic construct
  source 1..2559: mol_type = other DNA
atgaagcgagcagctgcaaagcatctaatagaacgctactaccaccagttaactgagggctgtggaaatgaagcctgcacgaatgagttttgtgcttcctgtccaacttttcttcgtatggataataatgcagcagctattaaagccctcgagctttataagattaatgcaaaactctgtgatcctcatccctccaagaaaggagcaagctcagcttaccttgagaactcgaaaggtgcccccaacaactcctgctctgagataaaaatgaacaagaaaggcgctagaattgattttaaagatgtgacttacttaacagaagagaaggtatatgaaattcttgaattatgtagagaaagagaggattattcccctttaatccgtgttattggaagagttttttctagtgctgaggcattggtacagagcttccggaaagttaaacaacacaccaaggaagaactgaaatctcttcaagcaaaagatgaagacaaagatgaagatgaaaaggaaaaagctgcatgttctgctgctgctatggaagaagactcagaagcatcttcctcaaggataggtgatagctcacagggagacaacaatttgcaaaaattaggccctgatgatgtgtctgtggatattgatgccattagaagggtctacaccagattgctctctaatgaaaaaattgaaactgcctttctcaatgcacttgtatatttgtcacctaacgtggaatgtgacttgacgtatcacaatgtatactctcgagatcctaattatctgaatttgttcattatcgtaatggagaatagaaatctccacagtcctgaatatctggaaatggctttgccattattttgcaaagcgatgagcaagctaccccttgcagcccaaggaaaactgatcagactgtggtctaaatacaatgcagaccagattcggagaatgatggagacatttcagcaacttattacttataaagtcataagcaatgaatttaacagtcgaaatctagtgaatgatgatgatgccattgttgctgcttcgaagtgcttgaaaatggtttactatgcaaatgtagtgggaggggaagtggacacaaatcacaatgaagaagatgatgaagagcccatccctgagtccagcgagctgacacttcaggaacttttgggagaagaaagaagaaacaagaaaggtcctcgagtggaccccctggaaactgaacttggtgttaaaaccctggattgtcgaaaaccacttatcccttttgaagagtttattaatgaaccactgaatgaggttctagaaatggataaagattatacttttttcaaagtagaaacagagaacaaattctcttttatgacatgtccctttatattgaatgctgtcacaaagaatttgggattatattatgacaatagaattcgcatgtacagtgaacgaagaatcactgttctctacagcttagttcaaggacagcagttgaatccatatttgagactcaaagttagacgtgaccatatcatagatgatgcacttgtccggctagagatgatcgctatggaaaatcctgcagacttgaagaagcagttgtatgtggaatttgaaggagaacaaggagttgatgagggaggtgtttccaaagaattttttcagctggttgtggaggaaatcttcaatccagatattggtatgttcacatacgatgaatctacaaaattgttttggtttaatccatcttcttttgaaactgagggtcagtttactctgattggcatagtactgggtctggctatttacaataactgtatactggatgtacattttcccatggttgtctacaggaagctaatggggaaaaaaggaacttttcgtgacttgggagactctcacccagttctatatcagagtttaaaagatttattggagtatgaagggaatgtggaagatgacatgatgatcactttccagatatcacagacagatctttttggtaacccaatgatgtatgatctaaaggaaaatggtgataaaattccaattacaaatgaaaacaggaaggaatttgtcaatctttattctgactacattctcaataaatcagtagaaaaacagttcaaggcttttcggagaggttttcatatggtgaccaatgaatctcccttaaagtacttattcagaccagaagaaattgaattgcttatatgtggaagccggaatctagatttccaagcactagaagaaactacagaatatgacggtggctataccagggactctgttctgattagggagttctgggaaatcgttcattcatttacagatgaacagaaaagactcttcttgcagtttacaacgggcacagacagagcacctgtgggaggactaggaaaattaaagatgattatagccaaaaatggcccagacacagaaaggttacctacatctcatacttgctttaatgtgcttttacttccggaatactcaagcaaagaaaaacttaaagagagattgttgaaggccatcacgtatgccaaaggatttggcatgctctaa

SEQ ID NO: 6 - DNA, 225 bp, synthetic construct
  source 1..225: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 7 - DNA, 600 bp, synthetic construct
  source 1..600: mol_type = other DNA
gtgaagtgggtaacctttatttcccttctttttctctttagctctgcttattccaggggtgtgtttaggagagatgcacacaagagtgaggttgctcataggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttagggaaacctatggtgaataggctgactgctgtgcaaaacaagaacctgagagaaatgaatgcttcttgcaacacaaagatgacaacccaaacctccccagattggtgagaccagaggttgatgtgtagtgcactgcttttcatgacaatgaagagacctttttgaaaaaatacttatatgaaattgccagaagacatccttacttttatgcccctgaactccttttctttgctaaaaggtataaagctgcttttacagaatgttgccaagctgctgataaagctgcc

SEQ ID NO: 8 - DNA, 145 bp, synthetic construct
  source 1..145: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 9 - DNA, 141 bp, synthetic construct
  source 1..141: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 10 - DNA, 141 bp, synthetic construct
  source 1..141: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 11 - DNA, 4671 bp, synthetic construct
  source 1..4671: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgcgtcgacagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagaagcttagggtgagtctatgggacccttgatgttttctttccccttcttttctatggttaagttcatgtcataggaaggggataagtaacagggtacacatattgaccaaatcagggtaattttgcatttgtaattttaaaaaatgctttcttcttttaatatacttttttgtttatcttatttctaatactttccctaatctctttctttcaaggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctccaccggtcgccaccatgaagcgagcagctgcaaagcatctaatagaacgctactaccaccagttaactgagggctgtggaaatgaagcctgcacgaatgagttttgtgcttcctgtccaacttttcttcgtatggataataatgcagcagctattaaagccctcgagctttataagattaatgcaaaactctgtgatcctcatccctccaagaaaggagcaagctcagcttaccttgagaactcgaaaggtgcccccaacaactcctgctctgagataaaaatgaacaagaaaggcgctagaattgattttaaagatgtgacttacttaacagaagagaaggtatatgaaattcttgaattatgtagagaaagagaggattattcccctttaatccgtgttattggaagagttttttctagtgctgaggcattggtacagagcttccggaaagttaaacaacacaccaaggaagaactgaaatctcttcaagcaaaagatgaagacaaagatgaagatgaaaaggaaaaagctgcatgttctgctgctgctatggaagaagactcagaagcatcttcctcaaggataggtgatagctcacagggagacaacaatttgcaaaaattaggccctgatgatgtgtctgtggatattgatgccattagaagggtctacaccagattgctctctaatgaaaaaattgaaactgcctttctcaatgcacttgtatatttgtcacctaacgtggaatgtgacttgacgtatcacaatgtatactctcgagatcctaattatctgaatttgttcattatcgtaatggagaatagaaatctccacagtcctgaatatctggaaatggctttgccattattttgcaaagcgatgagcaagctaccccttgcagcccaaggaaaactgatcagactgtggtctaaatacaatgcagaccagattcggagaatgatggagacatttcagcaacttattacttataaagtcataagcaatgaatttaacagtcgaaatctagtgaatgatgatgatgccattgttgctgcttcgaagtgcttgaaaatggtttactatgcaaatgtagtgggaggggaagtggacacaaatcacaatgaagaagatgatgaagagcccatccctgagtccagcgagctgacacttcaggaacttttgggagaagaaagaagaaacaagaaaggtcctcgagtggaccccctggaaactgaacttggtgttaaaaccctggattgtcgaaaaccacttatcccttttgaagagtttattaatgaaccactgaatgaggttctagaaatggataaagattatacttttttcaaagtagaaacagagaacaaattctcttttatgacatgtccctttatattgaatgctgtcacaaagaatttgggattatattatgacaatagaattcgcatgtacagtgaacgaagaatcactgttctctacagcttagttcaaggacagcagttgaatccatatttgagactcaaagttagacgtgaccatatcatagatgatgcacttgtccggctagagatgatcgctatggaaaatcctgcagacttgaagaagcagttgtatgtggaatttgaaggagaacaaggagttgatgagggaggtgtttccaaagaattttttcagctggttgtggaggaaatcttcaatccagatattggtatgttcacatacgatgaatctacaaaattgttttggtttaatccatcttcttttgaaactgagggtcagtttactctgattggcatagtactgggtctggctatttacaataactgtatactggatgtacattttcccatggttgtctacaggaagctaatggggaaaaaaggaacttttcgtgacttgggagactctcacccagttctatatcagagtttaaaagatttattggagtatgaagggaatgtggaagatgacatgatgatcactttccagatatcacagacagatctttttggtaacccaatgatgtatgatctaaaggaaaatggtgataaaattccaattacaaatgaaaacaggaaggaatttgtcaatctttattctgactacattctcaataaatcagtagaaaaacagttcaaggcttttcggagaggttttcatatggtgaccaatgaatctcccttaaagtacttattcagaccagaagaaattgaattgcttatatgtggaagccggaatctagatttccaagcactagaagaaactacagaatatgacggtggctataccagggactctgttctgattagggagttctgggaaatcgttcattcatttacagatgaacagaaaagactcttcttgcagtttacaacgggcacagacagagcacctgtgggaggactaggaaaattaaagatgattatagccaaaaatggcccagacacagaaaggttacctacatctcatacttgctttaatgtgcttttacttccggaatactcaagcaaagaaaaacttaaagagagattgttgaaggccatcacgtatgccaaaggatttggcatgctctaagtttaaacgattcgaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtgaagtgggtaacctttatttcccttctttttctctttagctctgcttattccaggggtgtgtttaggagagatgcacacaagagtgaggttgctcataggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttagggaaacctatggtgaataggctgactgctgtgcaaaacaagaacctgagagaaatgaatgcttcttgcaacacaaagatgacaacccaaacctccccagattggtgagaccagaggttgatgtgtagtgcactgcttttcatgacaatgaagagacctttttgaaaaaatacttatatgaaattgccagaagacatccttacttttatgcccctgaactccttttctttgctaaaaggtataaagctgcttttacagaatgttgccaagctgctgataaagctgccggcgcgccatcgatgaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 12 - DNA, 2559 bp, synthetic construct
  source 1..2559: mol_type = other DNA
atgaagcgagcagctgcaaagcatctaatagaacgctactaccaccagttaactgagggctgtggaaatgaagcctgcacgaatgagttttgtgcttcctgtccaacttttcttcgtatggataataatgcagcagctattaaagccctcgagctttataagattaatgcaaaactctgtgatcctcatccctccaagaaaggagcaagctcagcttaccttgagaactcgaaaggtgcccccaacaactcctgctctgagataaaaatgaacaagaaaggcgctagaattgattttaaagatgtgacttacttaacagaagagaaggtatatgaaattcttgaattatgtagagaaagagaggattattcccctttaatccgtgttattggaagagttttttctagtgctgaggcattggtacagagcttccggaaagttaaacaacacaccaaggaagaactgaaatctcttcaagcaaaagatgaagacaaagatgaagatgaaaaggaaaaagctgcatgttctgctgctgctatggaagaagactcagaagcatcttcctcaaggataggtgatagctcacagggagacaacaatttgcaaaaattaggccctgatgatgtgtctgtggatattgatgccattagaagggtctacaccagattgctctctaatgaaaaaattgaaactgcctttctcaatgcacttgtatatttgtcacctaacgtggaatgtgacttgacgtatcacaatgtatactctcgagatcctaattatctgaatttgttcattatcgtaatggagaatagaaatctccacagtcctgaatatctggaaatggctttgccattattttgcaaagcgatgagcaagctaccccttgcagcccaaggaaaactgatcagactgtggtctaaatacaatgcagaccagattcggagaatgatggagacatttcagcaacttattacttataaagtcataagcaatgaatttaacagtcgaaatctagtgaatgatgatgatgccattgttgctgcttcgaagtgcttgaaaatggtttactatgcaaatgtagtgggaggggaagtggacacaaatcacaatgaagaagatgatgaagagcccatccctgagtccagcgagctgacacttcaggaacttttgggagaagaaagaagaaacaagaaaggtcctcgagtggaccccctggaaactgaacttggtgttaaaaccctggattgtcgaaaaccacttatcccttttgaagagtttattaatgaaccactgaatgaggttctagaaatggataaagattatacttttttcaaagtagaaacagagaacaaattctcttttatgacatgtccctttatattgaatgctgtcacaaagaatttgggattatattatgacaatagaattcgcatgtacagtgaacgaagaatcactgttctctacagcttagttcaaggacagcagttgaatccatatttgagactcaaagttagacgtgaccatatcatagatgatgcacttgtccggctagagatgatcgctatggaaaatcctgcagacttgaagaagcagttgtatgtggaatttgaaggagaacaaggagttgatgagggaggtgtttccaaagaattttttcagctggttgtggaggaaatcttcaatccagatattggtatgttcacatacgatgaatctacaaaattgttttggtttaatccatcttcttttgaaactgagggtcagtttactctgattggcatagtactgggtctggctatttacaataactgtatactggatgtacattttcccatggttgtctacaggaagctaatggggaaaaaaggaacttttcgtgacttgggagactctcacccagttctatatcagagtttaaaagatttattggagtatgaagggaatgtggaagatgacatgatgatcactttccagatatcacagacagatctttttggtaacccaatgatgtatgatctaaaggaaaatggtgataaaattccaattacaaatgaaaacaggaaggaatttgtcaatctttattctgactacattctcaataaatcagtagaaaaacagttcaaggcttttcggagaggttttcatatggtgaccaatgaatctcccttaaagtacttattcagaccagaagaaattgaattgcttatatgtggaagccggaatctagatttccaagcactagaagaaactacagaatatgacggtggctataccagggactctgttctgattagggagttctgggaaatcgttcattcatttacagatgaacagaaaagactcttcttgcagtttacaacgggcacagacagagcacctgtgggaggactaggaaaattaaagatgattatagccaaaaatggcccagacacagaaaggttacctacatctcatacttgctttaatgtgcttttacttccggaatactcaagcaaagaaaaacttaaagagagattgttgaaggccatcacgtatgccaaaggatttggcatgctgtaa

SEQ ID NO: 13 - DNA, 476 bp, synthetic construct
  source 1..476: mol_type = other DNA
gtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 14 - DNA, 2165 bp, synthetic construct
  source 1..2165: mol_type = other DNA
gcgcgccatcgatgtgaagtgggtaacctttatttcccttctttttctctttagctcggcttattccaggggtgtgtttcgtcgagatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacctttttgaaaaaatacttatacgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacctatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgtcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacctttttgaaaaaatacttatacgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagacttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgc

SEQ ID NO: 15 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIGVSLGGGQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 16 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTRQDQPINAQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSSKVESWTEWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 17 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQNAVGALSTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 18 - DNA, 301 bp, synthetic construct
  source 1..301: mol_type = other DNA
tacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 19 - protein, 852 aa, synthetic construct
MKRAAAKHLIERYYHQLTEGCGNEACTNEFCASCPTFLRMDNNAAAIKALELYKINAKLCDPHPSKKGASSAYLENSKGAPNNSCSEIKMNKKGARIDFKDVTYLTEEKVYEILELCREREDYSPLIRVIGRVFSSAEALVQSFRKVKQHTKEELKSLQAKDEDKDEDEKEKAACSAAAMEEDSEASSSRIGDSSQGDNNLQKLGPDDVSVDIDAIRRVYTRLLSNEKIETAFLNALVYLSPNVECDLTYHNVYSRDPNYLNLFIIVMENRNLHSPEYLEMALPLFCKAMSKLPLAAQGKLIRLWSKYNADQIRRMMETFQQLITYKVISNEFNSRNLVNDDDAIVAASKCLKMVYYANVVGGEVDTNHNEEDDEEPIPESSELTLQELLGEERRNKKGPRVDPLETELGVKTLDCRKPLIPFEEFINEPLNEVLEMDKDYTFFKVETENKFSFMTCPFILNAVTKNLGLYYDNRIRMYSERRITVLYSLVQGQQLNPYLRLKVRRDHIIDDALVRLEMIAMENPADLKKQLYVEFEGEQGVDEGGVSKEFFQLVVEEIFNPDIGMFTYDESTKLFWFNPSSFETEGQFTLIGIVLGLAIYNNCILDVHFPMVVYRKLMGKKGTFRDLGDSHPVLYQSLKDLLEYEGNVEDDMMITFQISQTDLFGNPMMYDLKENGDKIPITNENRKEFVNLYSDYILNKSVEKQFKAFRRGFHMVTNESPLKYLFRPEEIELLICGSRNLDFQALEETTEYDGGYTRDSVLIREFWEIVHSFTDEQKRLFLQFTTGTDRAPVGGLGKLKMIIAKNGPDTERLPTSHTCFNVLLLPEYSSKEKLKERLLKAITYAKGFGML

SEQ ID NO: 20 - DNA, 122 bp, synthetic construct
  source 1..122: mol_type = other DNA
taagatacattgatgagtttggacaaaccacaactagaatgcagtgaaaaaaatgctttatttgtgaaatttgtgatgctattgctttatttgtaaccattataagctgcaataaacaagtt

SEQ ID NO: 21 - DNA, 56 bp, synthetic construct
  source 1..56: mol_type = other DNA
aataaaggaaatttattttcattgcaatagtgtgttggaattttttgtgtctctca

SEQ ID NO: 22 - DNA, 6202 bp, synthetic construct
  source 1..6202: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcacgatatcgggtccccaattgacattattgaagcatttatcagggttattgtctcagaatttaaatgacgcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgcgtcgacagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagatgcatgtaagtttagtctttttgtcttttatttcaggtcccggatccggtggtggtgcaaatcaaagaactgctcctcagtggatgttgcctttacttctagcaccggtcgccaccatgaagcgagcagctgcaaagcatctaatagaacgctactaccaccagttaactgagggctgtggaaatgaagcctgcacgaatgagttttgtgcttcctgtccaacttttcttcgtatggataataatgcagcagctattaaagccctcgagctttataagattaatgcaaaactctgtgatcctcatccctccaagaaaggagcaagctcagcttaccttgagaactcgaaaggtgcccccaacaactcctgctctgagataaaaatgaacaagaaaggcgctagaattgattttaaagatgtgacttacttaacagaagagaaggtatatgaaattcttgaattatgtagagaaagagaggattattcccctttaatccgtgttattggaagagttttttctagtgctgaggcattggtacagagcttccggaaagttaaacaacacaccaaggaagaactgaaatctcttcaagcaaaagatgaagacaaagatgaagatgaaaaggaaaaagctgcatgttctgctgctgctatggaagaagactcagaagcatcttcctcaaggataggtgatagctcacagggagacaacaatttgcaaaaattaggccctgatgatgtgtctgtggatattgatgccattagaagggtctacaccagattgctctctaatgaaaaaattgaaactgcctttctcaatgcacttgtatatttgtcacctaacgtggaatgtgacttgacgtatcacaatgtatactctcgagatcctaattatctgaatttgttcattatcgtaatggagaatagaaatctccacagtcctgaatatctggaaatggctttgccattattttgcaaagcgatgagcaagctaccccttgcagcccaaggaaaactgatcagactgtggtctaaatacaatgcagaccagattcggagaatgatggagacatttcagcaacttattacttataaagtcataagcaatgaatttaacagtcgaaatctagtgaatgatgatgatgccattgttgctgcttcgaagtgcttgaaaatggtttactatgcaaatgtagtgggaggggaagtggacacaaatcacaatgaagaagatgatgaagagcccatccctgagtccagcgagctgacacttcaggaacttttgggagaagaaagaagaaacaagaaaggtcctcgagtggaccccctggaaactgaacttggtgttaaaaccctggattgtcgaaaaccacttatcccttttgaagagtttattaatgaaccactgaatgaggttctagaaatggataaagattatacttttttcaaagtagaaacagagaacaaattctcttttatgacatgtccctttatattgaatgctgtcacaaagaatttgggattatattatgacaatagaattcgcatgtacagtgaacgaagaatcactgttctctacagcttagttcaaggacagcagttgaatccatatttgagactcaaagttagacgtgaccatatcatagatgatgcacttgtccggctagagatgatcgctatggaaaatcctgcagacttgaagaagcagttgtatgtggaatttgaaggagaacaaggagttgatgagggaggtgtttccaaagaattttttcagctggttgtggaggaaatcttcaatccagatattggtatgttcacatacgatgaatctacaaaattgttttggtttaatccatcttcttttgaaactgagggtcagtttactctgattggcatagtactgggtctggctatttacaataactgtatactggatgtacattttcccatggttgtctacaggaagctaatggggaaaaaaggaacttttcgtgacttgggagactctcacccagttctatatcagagtttaaaagatttattggagtatgaagggaatgtggaagatgacatgatgatcactttccagatatcacagacagatctttttggtaacccaatgatgtatgatctaaaggaaaatggtgataaaattccaattacaaatgaaaacaggaaggaatttgtcaatctttattctgactacattctcaataaatcagtagaaaaacagttcaaggcttttcggagaggttttcatatggtgaccaatgaatctcccttaaagtacttattcagaccagaagaaattgaattgcttatatgtggaagccggaatctagatttccaagcactagaagaaactacagaatatgacggtggctataccagggactctgttctgattagggagttctgggaaatcgttcattcatttacagatgaacagaaaagactcttcttgcagtttacaacgggcacagacagagcacctgtgggaggactaggaaaattaaagatgattatagccaaaaatggcccagacacagaaaggttacctacatctcatacttgctttaatgtgcttttacttccggaatactcaagcaaagaaaaacttaaagagagattgttgaaggccatcacgtatgccaaaggatttggcatgctgtaagtttaaacaagctttaagatacattgatgagtttggacaaaccacaactagaatgcagtgaaaaaaatgctttatttgtgaaatttgtgatgctattgctttatttgtaaccattataagctgcaataaacaagttctcgagccatgggcgcgccatcgatgaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggctgatttaaatcctgcaggcatttaattaagcaagctgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccggccggccaatgcctgacgatgcgtggagacctgtacaggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacgggccagagctgca

SEQ ID NO: 23 - DNA, 6996 bp, synthetic construct
  source 1..6996: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcacgatatcgggtccccaattgacattattgaagcatttatcagggttattgtctcagaatttaaatgacgcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgcgtcgacacacacacacccacacacttggctatacaaaaaagatgttccgagactgacagttaaaaattacactgctggccgagcactgtggctcatgcctgtagtcccaaaactttgggaggccgaggtgggaggatcacttgaggccaggagctcgagaccagcctgggcctaacatagcaagacaccatttctaagtttaaaaataaaaataaatttaaaaaagatacactgctatgtacgactgttgatataaaaaatctgaatactagacatgggtactcatctagtacttcaagggcttatcaacaaagttgcaagttgtaacactatgaattgttagtgatactcttttggctttgctagcaagtgttgtaaagctatacacacacacacacacacacacacacatacacacacacaccttttaaaatggtgaccctggtaccaaatatgactttaaatggatttaattttaatggctttaactacgttcagctgtcatatggatcaaaattagcctctatccagctggggtcaaccagggagccacttttcttaaccgacgacctactgaacgtcaacaactgcaggagacgggactttaccttcgtctctggtaaactagttgacacatcctgtgttggcaagaggcctaagtagatgaccttggtcctctaaaatctggcctgcactctcggggcacccctgcaacatctacaaaggcagctccagatagaaaagggttggggtcgaaaagccaataacggcaggcacctgccccgcctcggggctggggggctattccagcggcttcagctaactttcagagccattcgtttcccaacaaagtctgaggcgttcctctgctgggtacaccaaggggctctgcaaccctcctgggggggggggtgcccagagggcttccggaagtcccaggtttattctttcgggtcacagacagcagaaactaaaaagagggattaccctttctgtccagtcgcaagatggcgaccgagcctggtgggactccgaggggccgcaggccacctcctcttcccaatggcccgtgcgccggcggcgacggcaagcgggagggaggcggggccggcgaaggaaggaggggcggagcgcggcgccctcccgcgcgtcttggccccgccccacgtccccgcgtcccggcctggagccctcgcccggccgggcggcgcgcgctgcctgccgggatactcggcccgccccaccggtatgcatgtaagtttagtctttttgtcttttatttcaggtcccggatccggtggtggtgcaaatcaaagaactgctcctcagtggatgttgcctttacttctagcaccggtcgccaccatgaagcgagcagctgcaaagcatctaatagaacgctactaccaccagttaactgagggctgtggaaatgaagcctgcacgaatgagttttgtgcttcctgtccaacttttcttcgtatggataataatgcagcagctattaaagccctcgagctttataagattaatgcaaaactctgtgatcctcatccctccaagaaaggagcaagctcagcttaccttgagaactcgaaaggtgcccccaacaactcctgctctgagataaaaatgaacaagaaaggcgctagaattgattttaaagatgtgacttacttaacagaagagaaggtatatgaaattcttgaattatgtagagaaagagaggattattcccctttaatccgtgttattggaagagttttttctagtgctgaggcattggtacagagcttccggaaagttaaacaacacaccaaggaagaactgaaatctcttcaagcaaaagatgaagacaaagatgaagatgaaaaggaaaaagctgcatgttctgctgctgctatggaagaagactcagaagcatcttcctcaaggataggtgatagctcacagggagacaacaatttgcaaaaattaggccctgatgatgtgtctgtggatattgatgccattagaagggtctacaccagattgctctctaatgaaaaaattgaaactgcctttctcaatgcacttgtatatttgtcacctaacgtggaatgtgacttgacgtatcacaatgtatactctcgagatcctaattatctgaatttgttcattatcgtaatggagaatagaaatctccacagtcctgaatatctggaaatggctttgccattattttgcaaagcgatgagcaagctaccccttgcagcccaaggaaaactgatcagactgtggtctaaatacaatgcagaccagattcggagaatgatggagacatttcagcaacttattacttataaagtcataagcaatgaatttaacagtcgaaatctagtgaatgatgatgatgccattgttgctgcttcgaagtgcttgaaaatggtttactatgcaaatgtagtgggaggggaagtggacacaaatcacaatgaagaagatgatgaagagcccatccctgagtccagcgagctgacacttcaggaacttttgggagaagaaagaagaaacaagaaaggtcctcgagtggaccccctggaaactgaacttggtgttaaaaccctggattgtcgaaaaccacttatcccttttgaagagtttattaatgaaccactgaatgaggttctagaaatggataaagattatacttttttcaaagtagaaacagagaacaaattctcttttatgacatgtccctttatattgaatgctgtcacaaagaatttgggattatattatgacaatagaattcgcatgtacagtgaacgaagaatcactgttctctacagcttagttcaaggacagcagttgaatccatatttgagactcaaagttagacgtgaccatatcatagatgatgcacttgtccggctagagatgatcgctatggaaaatcctgcagacttgaagaagcagttgtatgtggaatttgaaggagaacaaggagttgatgagggaggtgtttccaaagaattttttcagctggttgtggaggaaatcttcaatccagatattggtatgttcacatacgatgaatctacaaaattgttttggtttaatccatcttcttttgaaactgagggtcagtttactctgattggcatagtactgggtctggctatttacaataactgtatactggatgtacattttcccatggttgtctacaggaagctaatggggaaaaaaggaacttttcgtgacttgggagactctcacccagttctatatcagagtttaaaagatttattggagtatgaagggaatgtggaagatgacatgatgatcactttccagatatcacagacagatctttttggtaacccaatgatgtatgatctaaaggaaaatggtgataaaattccaattacaaatgaaaacaggaaggaatttgtcaatctttattctgactacattctcaataaatcagtagaaaaacagttcaaggcttttcggagaggttttcatatggtgaccaatgaatctcccttaaagtacttattcagaccagaagaaattgaattgcttatatgtggaagccggaatctagatttccaagcactagaagaaactacagaatatgacggtggctataccagggactctgttctgattagggagttctgggaaatcgttcattcatttacagatgaacagaaaagactcttcttgcagtttacaacgggcacagacagagcacctgtgggaggactaggaaaattaaagatgattatagccaaaaatggcccagacacagaaaggttacctacatctcatacttgctttaatgtgcttttacttccggaatactcaagcaaagaaaaacttaaagagagattgttgaaggccatcacgtatgccaaaggatttggcatgctgtaagtttaaacaagctttaagatacattgatgagtttggacaaaccacaactagaatgcagtgaaaaaaatgctttatttgtgaaatttgtgatgctattgctttatttgtaaccattataagctgcaataaacaagttctcgagccatgggcgcgccatcgatgaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggctgatttaaatcctgcaggcatttaattaagcaagctgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccggccggccaatgcctgacgatgcgtggagacctgtacaggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacgggccagagctgca

SEQ ID NO: 24 - DNA, 1235 bp, synthetic construct
  source 1..1235: mol_type = other DNA
acacacacacccacacacttggctatacaaaaaagatgttccgagactgacagttaaaaattacactgctggccgagcactgtggctcatgcctgtagtcccaaaactttgggaggccgaggtgggaggatcacttgaggccaggagctcgagaccagcctgggcctaacatagcaagacaccatttctaagtttaaaaataaaaataaatttaaaaaagatacactgctatgtacgactgttgatataaaaaatctgaatactagacatgggtactcatctagtacttcaagggcttatcaacaaagttgcaagttgtaacactatgaattgttagtgatactcttttggctttgctagcaagtgttgtaaagctatacacacacacacacacacacacacacatacacacacacaccttttaaaatggtgaccctggtaccaaatatgactttaaatggatttaattttaatggctttaactacgttcagctgtcatatggatcaaaattagcctctatccagctggggtcaaccagggagccacttttcttaaccgacgacctactgaacgtcaacaactgcaggagacgggactttaccttcgtctctggtaaactagttgacacatcctgtgttggcaagaggcctaagtagatgaccttggtcctctaaaatctggcctgcactctcggggcacccctgcaacatctacaaaggcagctccagatagaaaagggttggggtcgaaaagccaataacggcaggcacctgccccgcctcggggctggggggctattccagcggcttcagctaactttcagagccattcgtttcccaacaaagtctgaggcgttcctctgctgggtacaccaaggggctctgcaaccctcctgggggggggggtgcccagagggcttccggaagtcccaggtttattctttcgggtcacagacagcagaaactaaaaagagggattaccctttctgtccagtcgcaagatggcgaccgagcctggtgggactccgaggggccgcaggccacctcctcttcccaatggcccgtgcgccggcggcgacggcaagcgggagggaggcggggccggcgaaggaaggaggggcggagcgcggcgccctcccgcgcgtcttggccccgccccacgtccccgcgtcccggcctggagccctcgcccggccgggcggcgcgcgctgcctgccgggatactcggcccgccc

(19) United States
(12) Patent Application Publication
(10) Pub. No.: US 2024/0261438 A1
(43) Pub. Date: Aug. 8, 2024

(54) GENE THERAPY FOR ANGELMAN SYNDROME

(71) Applicant: Ginkgo Bioworks, Inc., Boston, MA (US)

(72) Inventors: Kevin Sheets, Wake Forest, NC (US); Erin Borchardt, Cary, NC (US)

(21) Appl. No.: 18/435,561

(22) Filed: Feb. 7, 2024

Related U.S. Application Data
(60) Provisional application No. 63/483,894, filed on Feb. 8, 2023.

(51) Int. Cl. A61K 48/00; A61K 38/53; A61P 25/28; C12N 7/00; C12N 15/86

(52) U.S. Cl. CPC A61K 48/0058; A61K 38/53; A61P 25/28; C12N 7/00; C12N 15/86; C12Y 603/02019; C12N 2750/14143; C12N 2750/14151

(57) ABSTRACT

The disclosure provides nucleic acids (comprising AAV expression cassettes), AAV vectors, and compositions for use in methods for treating and/or delaying the onset of diseases associated with mutations in genes, such as UBE3A, associated with Angelman syndrome. Also, provided herein are methods for treating and/or delaying the onset of Angelman syndrome.

### CROSS-REFERENCE TO RELATED APPLICATIONS

This application claims priority to U.S. provisional application No. 63/483,894 filed on Feb. 8, 2023, which is herein incorporated by reference in its entirety for all purposes.

### REFERENCE TO AN ELECTRONIC SEQUENCE LISTING

The contents of the electronic sequence listing (STRD_028_00 US_SeqList_ST26.xml; Size: 58,915 bytes; and Date of Creation: Feb. 6, 2024) are herein incorporated by reference in its entirety.

### BACKGROUND

Angelman syndrome is a genetic disorder, characterized by developmental delay, intellectual disability, speech impairment, difficulty in walking, frequent smiling and laughing, excitability, and trouble going to sleep. Other symptoms of the syndrome include seizures, jerky movements, microcephaly, tongue thrusting, hand flapping and curved spine. While developmental delays due to Angelman syndrome may be first noted at around 6 months of age, the clinical features of the syndrome are usually detectable around or after one year of age.

Angelman syndrome is usually caused by lack of function of the maternally inherited ubiquitin protein ligase E3A (UBE3A). The gene encoding UBE3A is located within a region of chromosome 15, known as 15q11-q13. Angelman syndrome is seen to be associated with genetic errors, such as, deletion or mutation of one or more nucleic acids of the UBE3A gene or a segment of chromosome 15, uniparental disomy, imprinting defect, or translocation, often resulting the maternal copy of UBE3A gene being absent or not functioning normally.

There is currently no cure for Angelman syndrome. Standard of care for Angelman syndrome focuses on managing the symptoms. Thus, there is a need for disease-modifying therapeutic compositions and methods to treat Angelman syndrome.

### SUMMARY

The disclosure provides nucleic acid molecules, comprising an adeno-associated virus (AAV) expression cassette, wherein the AAV expression cassette comprises, from 5′ to 3′: (i) a 5′ AAV inverted terminal repeat (ITR); (ii) a promoter; (iii) an Angelman syndrome-associated transgene; and (iv) a 3′ AAV ITR. In some embodiments, the promoter drives expression of the Angelman syndrome-associated transgene. In some embodiments, the promoter is capable of expressing the transgene in a neuronal cell. In some embodiments, the promoter comprises a synapsin (SYN) promoter. In some embodiments, the SYN promoter comprises a nucleic acid sequence derived from: (i) a human SYN promoter, (ii) a chicken SYN promoter, (iii) a mouse SYN promoter, or (iv) any combination thereof. In some embodiments, the SYN promoter comprises a human SYN (hSYN) promoter.

In some embodiments, the hSYN promoter comprises the nucleic acid sequence SEQ ID NO: 3, or a sequence at least 90% identical thereto. In some embodiments, the Angelman syndrome-associated transgene encodes a ubiquitin protein ligase E3A (UBE3A). In some embodiments, the Angelman syndrome-associated transgene encodes a human UBE3A (hUBE3A). In some embodiments, the Angelman syndrome-associated transgene comprises a mutation capable of removing a predicted cryptic splice site. In some embodiments, the Angelman syndrome-associated transgene comprises a nucleic acid substitution of G2556C, relative to the nucleic acid sequence of wild type human UBE3A gene. In some embodiments, the Angelman syndrome-associated transgene comprises a nucleic acid sequence having at least 90% identity of SEQ ID NO: 12, and a nucleic acid substitution of G2556C, relative to SEQ ID NO: 12. In some embodiments, the Angelman syndrome-associated transgene comprises a nucleic acid sequence having at least 90% identity to SEQ ID NO: 5. In some embodiments, the Angelman syndrome-associated transgene comprises a nucleic acid sequence having at least 90% identity of SEQ ID NO: 5, and a nucleic acid substitution of G2556C, relative to SEQ ID NO: 12.

In some embodiments, at least one of the 5′ ITR and the 3′ ITR is about 110 to about 160 nucleotides in length. In some embodiments, the 5′ ITR is the same length as the 3′ ITR. In some embodiments, the 5′ ITR and the 3′ ITR are each about 145 nucleotides in length. In some embodiments, the 5′ ITR and the 3′ ITR are each about 141 nucleotides in length. In some embodiments, at least one of the 5′ ITR and the 3′ ITR is isolated or derived from the genome of AAV1, AAV2, AAV3, AAV4, AAV5, AAV6, AAV7, AAV8, AAV9, AAV10, AAV11, AAV12, AAVrh8, AAVrh10, AAVrh32.33, AAVrh74, Avian AAV or Bovine AAV. In some embodiments, the 5′ ITR and the 3′ ITR are each isolated or derived from the genome of AAV2. In some embodiments, the 5′ ITR comprises the sequence of SEQ ID NO: 2 or SEQ ID NO: 9. In some embodiments, the 3′ ITR comprises the sequence of SEQ ID NO: 8 or SEQ ID NO: 10.

In some embodiments, the AAV expression cassette comprises an intron. In some embodiments, the intron is derived from the human beta-globin gene (hBGIN). In some embodiments, the intron comprises one or more of the following mutations relative to SEQ ID NO: 13: (i) mutation at the 5′ terminus to contain Exon 2 splicing donor (AGG), (ii) mutation at the 3′ terminus to contain Exon 3 splicing acceptor (CTC), and (iii) G74T and G205A. In some embodiments, the intron comprises a nucleic acid sequence of SEQ ID NO: 4, or a sequence at least 90% identical thereto.

In some embodiments, the AAV expression cassette comprises a polyadenylation signal. In some embodiments, the polyadenylation signal is a polyadenylation signal isolated or derived from one or more of the following genes: simian virus 40 (SV40), rBG, α-globin, β-globin, human collagen, human growth hormone (hGH), polyoma virus, human growth hormone (hGH) or bovine growth hormone (bGH). In some embodiments, the AAV expression cassette comprises a bGH polyadenylation signal. In some embodiments, the bGH polyadenylation signal comprises a nucleic acid sequence of SEQ ID NO: 6, or a sequence at least 90% identical thereto.

In some embodiments, the AAV expression cassette comprises at least one stuffer sequence. In some embodiments, the at least one stuffer sequence comprises a nucleic acid sequence of SEQ ID NO: 7, or a sequence at least 90% identical thereto. In some embodiments, the AAV expression cassette comprises a Kozak sequence. In some embodiments, the Kozak sequence comprises the nucleic acid sequence of SEQ ID NO: 14, or a sequence at least 90% identical thereto; or the nucleic acid sequence of acagccacc, or a sequence at least 90% identical thereto. In some embodiments, the AAV expression cassette comprises an enhancer.

In some embodiments, the AAV expression cassette comprises a nucleic acid sequence SEQ ID NO: 1, or a sequence at least 90% identical thereto. In some embodiments, the AAV expression cassette comprises a nucleic acid sequence SEQ ID NO: 11, or a sequence at least 90% identical thereto.

The disclosure also provides plasmids, comprising any one of the nucleic acid molecules disclosed herein, and cells comprising any one of the nucleic acid molecules disclosed herein or any one of the plasmids disclosed herein. The disclosure further provides methods of producing a recombinant AAV vector, the method comprising contacting an AAV producer cell with any one of the nucleic acid molecules disclosed herein or any one of the plasmids disclosed herein. The disclosure provides recombinant AAV vectors produced by any one of the methods of producing a recombinant AAV vector disclosed herein.

In some embodiments, the vector is of a serotype selected from AAV1, AAV2, AAV3, AAV4, AAV5, AAV6, AAV7, AAV8, AAV9, AAV10, AAV11, AAV12, AAVrh8, AAVrh10, AAVrh32.33, AAVrh74, Avian AAV and Bovine AAV. In some embodiments, the recombinant AAV vector is a single-stranded AAV (ssAAV). In some embodiments, the recombinant AAV vector is a self-complementary AAV (scAAV). In some embodiments, the AAV vector comprises a capsid protein of AAV1, AAV2, AAV3, AAV4, AAV5, AAV6, AAV7, AAV8, AAV9, AAV10, AAV11, AAV12, AAVrh8, AAVrh10, AAVrh32.33, AAVrh74, Avian AAV or Bovine AAV. In some embodiments, the AAV vector comprises a capsid protein with one or more substitutions or mutations, as compared to a wild type AAV capsid protein. In some embodiments, the AAV vector comprises a capsid protein comprising: (i) the amino acid sequence of SEQ ID NO: 15, or a sequence at least 90% identical thereto, or (ii) the amino acid sequence of SEQ ID NO: 16, or a sequence at least 90% identical thereto, or (iii) the amino acid sequence of SEQ ID NO: 17, or a sequence at least 90% identical thereto.

In some embodiments, the AAV vector comprises a capsid protein comprising the amino acid sequence of SEQ ID NO: 15, or a sequence at least 90% identical thereto. In some embodiments, the AAV vector comprises a capsid protein comprising the amino acid sequence of SEQ ID NO: 15. In some embodiments, the AAV vector comprises a capsid protein comprising the amino acid sequence of SEQ ID NO: 16, or a sequence at least 90% identical thereto. In some embodiments, the AAV vector comprises a capsid protein comprising the amino acid sequence of SEQ ID NO: 16. In some embodiments, the AAV vector comprises a capsid protein comprising the amino acid sequence of SEQ ID NO: 17, or a sequence at least 90% identical thereto. In some embodiments, the AAV vector comprises a capsid protein comprising the amino acid sequence of SEQ ID NO: 17.

The disclosure provides compositions, comprising: (a) any one of the nucleic acid molecules disclosed herein, any one of the plasmids disclosed herein, any one of the cells disclosed herein, or any one of the recombinant AAV vectors disclosed herein; and (b) a pharmaceutically acceptable carrier. The disclosure provides methods of expressing an Angelman syndrome-associated transgene in a tissue, comprising: contacting the tissue with any one of the nucleic acid molecules disclosed herein, any one of the plasmids disclosed herein, any one of the recombinant AAV vectors disclosed herein, or any one of the compositions disclosed herein, thereby expressing the Angelman syndrome-associated transgene in the tissue.

In some embodiments, the tissue comprises brain tissue. In some embodiments, the tissue comprises neuronal cells. In some embodiments, the contacting step is performed in vitro, ex vivo, or in vivo. In some embodiments, the contacting step is performed in vivo in a subject in need thereof. In some embodiments, the contacting step comprises administering a therapeutically effective amount of the nucleic acid molecule, the plasmid, the recombinant AAV vector, or the composition to the subject. In some embodiments, the subject suffers from, or is at a risk of developing, the Angelman syndrome.

The disclosure provides methods for treating Angelman syndrome in a subject in need thereof, comprising administering to the subject a therapeutically effective amount of any one of the nucleic acid molecules disclosed herein, any one of the plasmids disclosed herein, any one of the cells disclosed herein, any one of the recombinant AAV vectors disclosed herein, or any one of the compositions disclosed herein, thereby treating Angelman syndrome in the subject. In some embodiments, the subject suffers from, or is at a risk of developing, the Angelman syndrome. In some embodiments, the Angelman syndrome is associated with, promoted by, or caused by a genetic mutation. In some embodiments, the genetic mutation comprises a mutation in the human UBE3A gene. In some embodiments, the genetic mutation comprises a mutation in the chromosomal region 15q11-q13.

In some embodiments, the method comprises diminishing the severity of; delaying the onset or progression of; and/or eliminating a symptom of the Angelman syndrome. In some embodiments, the symptom of the Angelman syndrome comprises: (a) developmental delay, (b) intellectual disability, (c) speech impairment, (d) gait ataxia, (e) tremulousness of the limbs, (f) frequent laughing or smiling, (g) excitability, (h) microcephaly, (i) seizures, (j) trouble sleeping, (k) tongue thrusting, (l) hand flapping, (m) curved spine or (n) any combination thereof. In some embodiments, the method comprises prolonging the survival of the subject, as compared to a control subject having Angelman syndrome, wherein the control subject has not been administered the therapeutically effective amount, or as compared to the expected survival of the subject prior to administration of the therapeutically effective amount. In some embodiments, the subject is a human subject.

These and other embodiments are addressed in more detail in the detailed description set forth below.

### BRIEF DESCRIPTION OF FIGURES

FIG. 1 shows a schematic representation of the AAV expression cassettes generated for the expression of the human ubiquitin protein ligase E3A (hUBE3A) gene.

FIG. 2 is a graph showing the hUBE3A mRNA expression levels in induced pluripotent stem cells (iPSCs) upon transduction of either wild type (WT) isogenic, healthy iPSCs and mutant (MU) UBE3^{-/+} iPSCs with the cassettes indicated on the X axis.

FIG. 3 is a graph showing the cell body cluster area of WT iPSCs and mutant (MU) UBE3^{-/+} iPSCs upon transduction with each of the cassettes or buffer, as indicated in the figure legend.

FIG. 4 is a graph showing the cell body cluster area of MU UBE3^{-/+} iPSCs 13 days after transduction with each of the cassettes, as indicated in the figure legend.

FIG. 5 is a graph showing the vector copy number (VCN; on the Y axis) in the tissues listed on the X axis (anterior brain, posterior brain and left lateral liver) upon administration of control vehicle or AAV particles comprising the AAV cassettes indicated in the figure legend and Table A to WT or Ube3a−/+ mice.

FIG. 6 is a graph showing the levels of UBE3A mRNA (on the Y axis) in the tissues listed on the X axis (anterior brain, posterior brain and left lateral liver) resulting from the expression of the hUBE3A gene upon administration of control vehicle or AAV particles comprising the AAV cassettes indicated in the figure legend and Table A to WT or Ube3a−/+ mice.

FIG. 7 is a Western Blot showing the expression of UBE3A protein (dotted box) in the anterior brain tissue resulting from the expression of the hUBE3A gene upon administration of control vehicle or AAV particles comprising the AAV cassettes indicated in the figure legend and Table A to WT or Ube3a−/+ mice.

FIG. 8 is a Western Blot showing the expression of UBE3A protein (dotted box) in the posterior brain tissue resulting from the expression of the hUBE3A gene upon administration of control vehicle or AAV particles comprising the AAV cassettes indicated in the figure legend and Table A to WT or Ube3a−/+ mice.

FIG. 9 is a graph showing the quantitation of the expression of UBE3A protein in the anterior brain, posterior brain or left lateral liver tissue, resulting from the expression of the hUBE3A gene upon administration of control vehicle or AAV particles comprising the AAV cassettes indicated in the figure legend and Table A to WT or Ube3a−/+ mice.

FIGS. 10A-10F are images from immunohistochemistry analysis of UBE3A anti-hUBE3A antibody staining of brain tissues obtained from WT or Ube3a−/+ mice upon administration of control vehicle or AAV particles comprising the AAV cassettes indicated in the figure legend and Table A.

FIG. 11 shows zoomed-in images from immunohistochemistry analysis of UBE3A using anti-hUBE3A antibody staining of brain tissues obtained from WT or Ube3a−/+ mice upon administration of control vehicle or AAV particles comprising the AAV cassettes indicated in the figure legend and Table A.

### DETAILED DESCRIPTION

The disclosure provides nucleic acids (comprising AAV expression cassettes), AAV vectors, and compositions for use in methods for treating and/or delaying the onset of diseases associated with mutations in genes, such as UBE3A, associated with Angelman syndrome. Also, provided herein are methods for treating and/or delaying the onset of Angelman syndrome.

### Definitions

The following terms are used in the description herein and the appended claims:

The singular forms “a,” “an” and “the” are intended to include the plural forms as well, unless the context clearly indicates otherwise.

Furthermore, the term “about” as used herein when referring to a measurable value such as an amount of the length of a polynucleotide or polypeptide sequence, dose, time, temperature, and the like, is meant to encompass variations of ±20%, ±10%, ±5%, ±1%, ±0.5%, or even±0.1% of the specified amount.

Also as used herein, “and/or” refers to and encompasses any and all possible combinations of one or more of the associated listed items, as well as the lack of combinations when interpreted in the alternative (“or”).

The term “wild type” is a term of the art understood by skilled persons and means the typical form of an organism, strain, gene, protein, or characteristic as it occurs in nature as distinguished from mutant or variant forms. For example, a wild type protein is the typical form of that protein as it occurs in nature.

The term “mutant protein” is a term of the art understood by skilled persons and refers to a protein that is distinguished from the wild type form of the protein on the basis of the presence of amino acid modifications, such as, for example, amino acid substitutions, insertions and/or deletions. The term “mutant gene” is a term of the art understood by skilled persons and refers to a gene that is distinguished from the wild type form of the gene on the basis of the presence of nucleic acid modifications, such as, for example, nucleic acid substitutions, insertions and/or deletions. In some embodiments, the mutant gene encodes a mutant protein.

A “nucleic acid” or “polynucleotide” is a sequence of nucleotide bases, for example RNA, DNA or DNA-RNA hybrid sequences (including both naturally occurring and non-naturally occurring nucleotides). In some embodiments, the nucleic acids of the disclosure are either single or double stranded DNA sequences. A nucleic acid may be 1-1,000, 1,000-10,000, 10,000-100,000, 100,000-1 million or greater than 1 million nucleotides in length. A nucleic acid will generally contain phosphodiester bonds, although in some cases nucleic acid analogs are included that may have alternate backbones, comprising, for example, phosphoramide, phosphorothioate, phosphorodithioate, O-methylphophoroamidite linkages, and peptide nucleic acid backbones and linkages. Other analog nucleic acids include those with positive backbones, non-ionic backbones, and non-ribose backbones. Nucleic acids containing one or more carbocyclic sugars are also included within the definition of nucleic acids. These modifications of the ribose-phosphate backbone may facilitate the addition of labels, or to increase the stability and half-life of such molecules in physiological environments. Nucleic acids of the disclosure may be linear, or may be circular (e.g., a plasmid).

As used herein, the term “promoter” refers to one or more nucleic acid control sequences that direct transcription of an operably linked nucleic acid. Promoters may include nucleic acid sequences near the start site of transcription, such as a TATA element. Promoters may also include cis-acting polynucleotide sequences that can be bound by transcription factors.

A “constitutive” promoter is a promoter that is active under most environmental and developmental conditions. An “inducible” promoter is a promoter that is active under environmental or developmental regulation. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

An “AAV expression cassette” is a nucleic acid that gets packaged into a recombinant AAV vector, and comprises a sequence encoding one or more transgenes. When the AAV vector is contacted with a target cell, the transgenes are expressed by the target cell.

As used herein, the terms “virus vector,” “viral vector,” or “gene delivery vector” refer to a virus particle that functions as a nucleic acid delivery vehicle, and which comprises a nucleic acid (e.g., an AAV expression cassette) packaged within a virion. Exemplary virus vectors of the disclosure include adenovirus vectors, adeno-associated virus vectors, lentivirus vectors, and retrovirus vectors.

As used herein, the term “adeno-associated virus” (AAV), includes but is not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, AAV type 12, AAV type 13, AAV type rh32.33, AAV type rh8, AAV type rh10, AAV type rh74, AAV type hu.68, avian AAV, bovine AAV, canine AAV, equine AAV, ovine AAV, snake AAV, bearded dragon AAV, AAV2i8, AAV2g9, AAV-LK03, AAV7m8, AAV Anc80, AAV PHP.B, and any other AAV now known or later discovered. See, e.g., Table 1.

TABLE 1
Adeno-Associated Virus Serotypes

| | | | GenBank | | GenBank |
|---|---|---|---|---|---|
| | | | Accession | | Accession |
| | GenBank Accession Number | | Number | | Number |
| Complete Genomes | | Clade C | | Rh57 | AY530569 |
| Adeno-associated virus 1 | NC_002077, AF063497 | Hu9 | AY530629 | Rh50 | AY530563 |
| Adeno-associated virus 2 | NC_001401 | Hu10 | AY530576 | Rh49 | AY530562 |
| Adeno-associated virus 3 | NC_001729 | Hu11 | AY530577 | Hu39 | AY530601 |
| Adeno-associated virus 3B | NC_001863 | Hu53 | AY530615 | Rh58 | AY530570 |
| Adeno-associated virus 4 | NC_001829 | Hu55 | AY530617 | Rh61 | AY530572 |
| Adeno-associated virus 5 | Y18065, AF085716 | Hu54 | AY530616 | Rh52 | AY530565 |
| Adeno-associated virus 6 | NC_001862, AAB95450.1 | Hu7 | AY530628 | Rh53 | AY530566 |
| Avian AAV ATCC VR-865 | AY186198, AY629583, NC_004828 | Hu18 | AY530583 | Rh51 | AY530564 |
| Avian AAV strain DA-1 | NC_006263, AY629583 | Hu15 | AY530580 | Rh64 | AY530574 |
| Bovine AAV | NC_005889, AY388617, AAR26465 | Hu16 | AY530581 | Rh43 | AY530560 |
| AAV11 | AAT46339, AY631966 | Hu25 | AY530591 | AAV8 | AF513852 |
| AAV12 | ABI16639, DQ813647 | Hu60 | AY530622 | Rh8 | AY242997 |
| Clade A | | Ch5 | AY243021 | Rh1 | AY530556 |
| AAV1 | NC_002077, AF063497 | Hu3 | AY530595 | Clade F | |
| AAV6 | NC_001862 | Hu1 | AY530575 | Hu14 (AAV9) | AY530579 |
| Hu.48 | AY530611 | Hu4 | AY530602 | Hu31 | AY530596 |
| Hu 43 | AY530606 | Hu2 | AY530585 | Hu32 | AY530597 |
| Hu 44 | AY530607 | Hu61 | AY530623 | HSC1 | MI332400.1 |
| Hu 46 | AY530609 | Clade D | | HSC2 | MI332401.1 |
| Clade B | | Rh62 | AY530573 | HSC3 | MI332402.1 |
| Hu. 19 | AY530584 | Rh48 | AY530561 | HSC4 | MI332403.1 |
| Hu. 20 | AY530586 | Rh54 | AY530567 | HSC5 | MI332405.1 |
| Hu 23 | AY530589 | Rh55 | AY530568 | HSC6 | MI332404.1 |
| Hu22 | AY530588 | Cy2 | AY243020 | HSC7 | MI332407.1 |
| Hu24 | AY530590 | AAV7 | AF513851 | HSC8 | MI332408.1 |
| Hu21 | AY530587 | Rh35 | AY243000 | HSC9 | MI332409.1 |
| Hu27 | AY530592 | Rh37 | AY242998 | HSC11 | MI332406.1 |
| Hu28 | AY530593 | Rh36 | AY242999 | HSC12 | MI332410.1 |
| Hu 29 | AY530594 | Cy6 | AY243016 | HSC13 | MI332411.1 |
| Hu63 | AY530624 | Cy4 | AY243018 | HSC14 | MI332412.1 |
| Hu64 | AY530625 | Cy3 | AY243019 | HSC15 | MI332413.1 |
| Hu13 | AY530578 | Cy5 | AY243017 | HSC16 | MI332414.1 |
| Hu56 | AY530618 | Rh13 | AY243013 | HSC17 | MI332415.1 |
| Hu57 | AY530619 | Clade E | | Hu68 | |
| Hu49 | AY530612 | Rh38 | AY530558 | Clonal Isolate | |
| Hu58 | AY530620 | Hu66 | AY530626 | AAV5 | Y18065, AF085716 |
| Hu34 | AY530598 | Hu42 | AY530605 | AAV 3 | NC_001729 |
| Hu35 | AY530599 | Hu67 | AY530627 | AAV 3B | NC_001863 |
| AAV2 | NC_001401 | Hu40 | AY530603 | AAV4 | NC_001829 |
| Hu45 | AY530608 | Hu41 | AY530604 | Rh34 | AY243001 |
| Hu47 | AY530610 | Hu37 | AY530600 | Rh33 | AY243002 |
| Hu51 | AY530613 | Rh40 | AY530559 | Rh32 | AY243003 |
| Hu52 | AY530614 | Rh2 | AY243007 | Others | |
| Hu T41 | AY695378 | Bb1 | AY243023 | Rh74 | |
| Hu S17 | AY695376 | Bb2 | AY243022 | Bearded Dragon AAV | |
| Hu T88 | AY695375 | Rh10 | AY243015 | Snake AAV | NC_006148.1 |
| Hu T71 | AY695374 | Hu17 | AY530582 | | |
| Hu T70 | AY695373 | Hu6 | AY530621 | | |
| Hu T40 | AY695372 | Rh25 | AY530557 | | |
| Hu T32 | AY695371 | Pi2 | AY530554 | | |
| Hu T17 | AY695370 | Pi1 | AY530553 | | |
| Hu LG15 | AY695377 | Pi3 | AY530555 | | |

The terms “viral production cell”, “viral production cell line,” or “viral producer cell” refer to cells used to produce viral vectors. HEK293 and 239T cells are common viral production cell lines. Table 2, below, lists exemplary viral production cell lines for various viral vectors.

TABLE 2
Exemplary viral production cell lines

| Virus Vector | Exemplary Viral Production Cell Line(s) |
|---|---|
| Adenovirus | HEK293, 911, pTG6559, PER.C6, GH329, |
| | N52.E6, HeLa-E1, UR, VLI-293 |
| Adeno-Associated Virus | HEK293, Sf9 |
| (AAV) | |
| Retrovirus | HEK293 |
| Lentivirus | 293T |

“HEK293” refers to a cell line originally derived from human embryonic kidney cells grown in tissue culture. The HEK293 cell line grows readily in culture, and is commonly used for viral production. As used herein, “HEK293” may also refer to one or more variant HEK293 cell lines, i.e., cell lines derived from the original HEK293 cell line that additionally comprise one or more genetic alterations. Many variant HEK293 lines have been developed and optimized for one or more particular applications. For example, the 293T cell line contains the SV40 large T-antigen that allows for episomal replication of transfected plasmids containing the SV40 origin of replication, leading to increased expression of desired gene products.

“Sf9” refers to an insect cell line that is a clonal isolate derived from the parental Spodoptera frugiperda cell line IPLB-Sf-21-AE. Sf9 cells can be grown in the absence of serum and can be cultured attached or in suspension.

A “transfection reagent” means a composition that enhances the transfer of nucleic acid into cells. Some transfection reagents commonly used in the art include one or more lipids that bind to nucleic acids and to the cell surface (e.g., Lipofectamine™).

As used herein “sequence identity” refers to the extent to which two optimally aligned polynucleotides or polypeptide sequences are invariant throughout a window of alignment of components, e.g., nucleotides or amino acids. An “identity fraction” for aligned segments of a test sequence and a reference sequence is the number of identical components which are shared by the two aligned sequences divided by the total number of components in the reference sequence segment, i.e., the entire reference sequence or a smaller defined part of the reference sequence. “Percent identity” is the identity fraction times 100. The extent of identity (homology) between two sequences can be ascertained using a computer program and mathematical algorithm. Percentage identity can be calculated using the alignment program Clustal Omega, available at www.ebi.ac.uk/Tools/msa/clustalo using default parameters. See, Sievers et al., “Fast, scalable generation of high-quality protein multiple sequence alignments using Clustal Omega.” (2011 Oct. 11) Molecular systems biology 7:539.

As used herein, “treatment” or “treating,” or “palliating” or “ameliorating” are used interchangeably. These terms refer to an approach for obtaining beneficial or desired results including but not limited to a therapeutic benefit and/or a prophylactic benefit. Therapeutic benefit refers to any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested.

The terms “subject,” “individual,” and “patient” are used interchangeably herein to refer to a vertebrate, such as a mammal. The mammal may be, for example, a mouse, a rat, a rabbit, a cat, a dog, a pig, a sheep, a horse, a non-human primate (e.g., cynomolgus monkey, chimpanzee), or a human. A subject's tissues, cells, or derivatives thereof, obtained in vivo or cultured in vitro are also encompassed. A human subject may be an adult, a teenager, a child (2 years to 14 years of age), an infant (1 month to 24 months), or a neonate (up to 1 month). In some embodiments, the adults are seniors about 65 years or older, or about 60 years or older. In some embodiments, the subject is a pregnant woman or a woman intending to become pregnant.

The term “effective amount” or “therapeutically effective amount” refers to the amount of an agent that is sufficient to achieve an outcome, for example, to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The specific dose may vary depending on one or more of: the particular agent chosen, the dosing regimen to be followed, whether it is administered in combination with other compounds, timing of administration, the tissue to be imaged, and the physical delivery system in which it is carried.

As used herein, the term “gene therapy” refers to the process of introducing genetic material into cells to compensate for abnormal genes, or to make a therapeutic protein.

### AAV Expression Cassettes

The disclosure provides nucleic acid sequences comprising one or more adeno-associated virus (AAV) expression cassettes. In some embodiments, the AAV expression cassette comprises a 5′ inverted terminal repeat (ITR), a promoter, a transgene, and a 3′ ITR. In some embodiments, the transgene is an Angelman syndrome-associated gene. In some embodiments, the AAV expression cassette comprises a Kozak sequence, a polyadenylation sequence, and/or a stuffer sequence.

In some embodiments, the AAV expression cassette comprises a nucleic acid sequence of SEQ ID NO: 1, or a sequence at least 70% identical thereto (for example, at least 75%, at least 80%, at least 85%, at least 90%, at least 95%, at least 96%, at least 97%, at least 98%, at least 99%, at least 99.5%, or 100% identical thereto, inclusive of all values and subranges that lie therebetween). In some embodiments, the AAV expression cassette comprises a nucleic acid sequence of SEQ ID NO: 1.

In some embodiments, the AAV expression cassette comprises a nucleic acid sequence of SEQ ID NO: 11, or a sequence at least 70% identical thereto (for example, at least 75%, at least 80%, at least 85%, at least 90%, at least 95%, at least 96%, at least 97%, at least 98%, at least 99%, at least 99.5%, or 100% identical thereto, inclusive of all values and subranges that lie therebetween). In some embodiments, the AAV expression cassette comprises a nucleic acid sequence of SEQ ID NO: 11.

### (i) Inverted Terminal Repeat

Inverted Terminal Repeat or ITR sequences are sequences that mediate AAV proviral integration and packaging of AAV DNA into virions. ITRs are involved in a variety of activities in the AAV life cycle. For example, the ITR sequences, which can form hairpin structures, play roles in excision from the plasmid after transfection, replication of the vector genome and integration and rescue from a host cell genome.

The AAV expression cassettes of the disclosure may comprise a 5′ ITR and a 3′ ITR. The ITR sequences may be about 110 to about 160 nucleotides in length, for example 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159 or 160 nucleotides in length. In some embodiments, the ITR sequences may be about 141 nucleotides in length. In some embodiments, the 5′ ITR is the same length as the 3′ ITR. In some embodiments, the 5′ ITR and the 3′ ITR have different lengths. In some embodiments, the 5′ ITR is longer than the 3′ ITR, and in other embodiments, the 3′ ITR is longer than the 5′ ITR.

The ITRs may be isolated or derived from the genome of any AAV, for example the AAVs listed in Table 1. In some embodiments, at least one of the 5′ ITR and the 3′ ITR is isolated or derived from the genome of AAV1, AAV2, AAV3, AAV4, AAV5, AAV6, AAV7, AAV8, AAV9, AAV10, AAV11, AAV12, AAVrh8, AAVrh10, AAVrh32.33, AAVrh74, Avian AAV or Bovine AAV. In some embodiments, at least one of the 5′ ITR and the 3′ITR may be a wild type or mutated ITR isolated or derived from a member of another parvovirus species besides AAV. For example, in some embodiments, an ITR may be a wild type or mutant ITR isolated or derived from bocavirus or parvovirus B19.

In some embodiments, the ITR comprises a modification to promote production of a scAAV. In some embodiments, the modification to promote production of a scAAV is deletion of the terminal resolution sequence (TRS) from the ITR. In some embodiments, the 5′ ITR is a wild type ITR, and the 3′ ITR is a mutated ITR lacking the terminal resolution sequence. In some embodiments, the 3′ ITR is a wild type ITR, and the 5′ ITR is a mutated ITR lacking the terminal resolution sequence. In some embodiments, the terminal resolution sequence is absent from both the 5′ ITR and the 3′ITR. In other embodiments, the modification to promote production of a scAAV is replacement of an ITR with a different hairpin-forming sequence, such as a short hairpin (sh)RNA-forming sequence.

In some embodiments, the 5′ ITR may comprise the sequence of SEQ ID NO: 2, or a sequence at least 70% identical thereto (for example, at least 75%, at least 80%, at least 85%, at least 90%, at least 95%, at least 96%, at least 97%, at least 98%, at least 99%, at least 99.5%, or 100% identical thereto, inclusive of all values and subranges that lie there between). In some embodiments, the 5′ ITR may comprise the sequence of SEQ ID NO: 9, or a sequence at least 70% identical thereto (for example, at least 75%, at least 80%, at least 85%, at least 90%, at least 95%, at least 96%, at least 97%, at least 98%, at least 99%, at least 99.5%, or 100% identical thereto, inclusive of all values and subranges that lie there between).

In some embodiments, the 3′ ITR may comprise the sequence of SEQ ID NO: 8, or a sequence at least 70% identical thereto (for example, at least 75%, at least 80%, at least 85%, at least 90%, at least 95%, at least 96%, at least 97%, at least 98%, at least 99%, at least 99.5%, or 100% identical thereto, inclusive of all values and subranges that lie there between). In some embodiments, the 3′ ITR may comprise the sequence of SEQ ID NO: 10, or a sequence at least 70% identical thereto (for example, at least 75%, at least 80%, at least 85%, at least 90%, at least 95%, at least 96%, at least 97%, at least 98%, at least 99%, at least 99.5%, or 100% identical thereto, inclusive of all values and subranges that lie there between).

In some embodiments, the 5′ ITR comprises the sequence of SEQ ID NO: 2, and the 3′ ITR comprises the sequence of SEQ ID NO: 8. In some embodiments, the 5′ ITR comprises the sequence of SEQ ID NO: 9, and the 3′ ITR comprises the sequence of SEQ ID NO: 10.

In some embodiments, the AAV expression cassettes comprise one or more “surrogate” ITRs, i.e., non-ITR sequences that serve the same function as ITRs. See, e.g., Xie, J. et al., Mol. Ther., 25(6): 1363-1374 (2017). In some embodiments, an ITR in an AAV expression cassette is replaced by a surrogate ITR. In some embodiments, the surrogate ITR comprises a hairpin-forming sequence. In some embodiments, the surrogate ITR is a shRNA-forming sequence.

### (ii) Promoters

In some embodiments, the AAV expression cassettes described herein comprise a promoter. In some embodiments, the promoter is a synthetic promoter. In some embodiments, the promoter may comprise a nucleic acid sequence derived from an endogenous promoter and/or an endogenous enhancer.

In some embodiments, the promoter comprises a nucleic acid sequence derived from one or more promoters commonly used in the art for gene expression. For instance, in some embodiments, the promoter further comprises a nucleic acid sequence derived from the CMV promoter, the SV40 early promoter, the SV40 late promoter, the metallothionein promoter, the murine mammary tumor virus (MMTV) promoter, the Rous sarcoma virus (RSV) promoter, the polyhedrin promoter, the chicken β-actin (CBA) promoter, the dihydrofolate reductase (DHFR) promoter, and the phosphoglycerol kinase (PGK) promoter. In some embodiments, the promoter comprises a nucleic acid sequence derived from the chicken β-actin (CBA) promoter, the EF-1 alpha promoter, or the EF-1 alpha short promoter.

In some embodiments, the promoter is capable of expressing the transgene in a neuronal cell. In some embodiments, the promoter is a cell-specific promoter, such as, a neuronal cell-specific promoter. As used herein, a “cell-specific promoter” refers to a promoter that is capable of expressing a transgene at a level that is higher in a particular cell (e.g., neuronal cell), as compared to a control cell (e.g., a non-neuronal cell). Therefore, in some embodiments, the AAV expression cassettes disclosed herein comprise a promoter that expresses the transgene in a neuronal cell at a level that is higher than a level of the transgene expression by the promoter in a non-neuronal cell. In some embodiments, the promoter expresses the transgene in a neuronal cell at a level that is at least about 1.2 fold (for example, about 1.5 fold, about 2 fold, about 2.5 fold, about 3 fold, about 3.5 fold, about 4 fold, about 4.5 fold, about 5 fold, about 5.5 fold, about 6 fold, about 6.5 fold, about 7 fold, about 7.5 fold, about 8 fold, about 8.5 fold, about 9 fold, about 9.5 fold, about 10 fold, about 15 fold, about 20 fold, about 30 fold, about 40 fold, about 50 fold, about 60 fold, about 70 fold, about 80 fold about 90 fold, or about 100 fold, including all values and subranges that lie therebetween) higher than a level of the transgene expression by the promoter in a non-neuronal cell.

In some embodiments, the promoter may comprise a nucleic acid sequence derived from an endogenous promoter and/or an endogenous enhancer, for example, an endogenous promoter and/or an endogenous enhancer of a gene that is expressed at higher levels in a neuronal cell, as compared to a non-neuronal cell.

In some embodiments, the promoter comprises a synapsin (SYN) promoter. In some embodiments, the SYN promoter comprises a nucleic acid sequence derived from: (i) a human SYN promoter, (ii) a chicken SYN promoter, (iii) a mouse SYN promoter, or (iv) any combination thereof. In some embodiments, the SYN promoter comprises a human SYN (hSYN) promoter.

In some embodiments, the promoter comprises the sequence of SEQ ID NO: 3, or a sequence at least 70% identical thereto (for example, at least 75%, at least 80%, at least 85%, at least 90%, at least 95%, at least 96%, at least 97%, at least 98%, at least 99%, at least 99.5%, or 100% identical thereto, inclusive of all values and subranges that lie there between).

In some embodiments, the AAV expression cassettes described herein further comprise an enhancer. The enhancer may be, for example, the CMV enhancer. In some embodiments, the enhancer comprises the sequence of SEQ ID NO: 18, or a sequence at least 70% identical thereto (for example, at least 75%, at least 80%, at least 85%, at least 90%, at least 95%, at least 96%, at least 97%, at least 98%, at least 99%, at least 99.5%, or 100% identical thereto, inclusive of all values and subranges that lie therebetween).

In some embodiments, the promoter further comprises a nucleic acid sequence derived from any one or more of the following promoters: HMG-COA reductase promoter; sterol regulatory element 1 (SRE-1); phosphoenol pyruvate carboxy kinase (PEPCK) promoter; human C-reactive protein (CRP) promoter; human glucokinase promoter; cholesterol 7-alpha hydroylase (CYP-7) promoter; beta-galactosidase alpha-2,6 sialyltransferase promoter; insulin-like growth factor binding protein (IGFBP-1) promoter; aldolase B promoter; human transferrin promoter; collagen type I promoter; prostatic acid phosphatase (PAP) promoter; prostatic secretory protein of 94 (PSP 94) promoter; prostate specific antigen complex promoter; human glandular kallikrein gene promoter (hgt-1); the myocyte-specific enhancer binding factor MEF-2; muscle creatine kinase promoter; pancreatitis associated protein promoter (PAP); elastase 1 transcriptional enhancer; pancreas specific amylase and elastase enhancer promoter; pancreatic cholesterol esterase gene promoter; uteroglobin promoter; cholesterol side-chain cleavage (SCC) promoter; gamma-gamma enolase (neuron-specific enolase, NSE) promoter; neurofilament heavy chain (NF-H) promoter; human CGL-1/granzyme B promoter; the terminal deoxy transferase (TdT), lambda 5, VpreB, and lck (lymphocyte specific tyrosine protein kinase p561ck) promoter; the humans CD2 promoter and its 3′ transcriptional enhancer; the human NK and T cell specific activation (NKG5) promoter; pp60c-src tyrosine kinase promoter; organ-specific neoantigens (OSNs), mw 40 kDa (p40) promoter; colon specific antigen-P promoter; human alpha-lactalbumin promoter; phosphoeholpyruvate carboxykinase (PEPCK) promoter, HER2/neu promoter, casein promoter, IgG promoter, Chorionic Embryonic Antigen promoter, elastase promoter, porphobilinogen deaminase promoter, insulin promoter, growth hormone factor promoter, tyrosine hydroxylase promoter, albumin promoter, alphafetoprotein promoter, acetyl-choline receptor promoter, alcohol dehydrogenase promoter, alpha or beta globin promoter, T-cell receptor promoter, the osteocalcin promoter the IL-2 promoter, IL-2 receptor promoter, whey (wap) promoter, and the MHC Class II promoter. In some embodiments, the AAV expression cassettes disclosed herein further comprise a nucleic acid sequence derived from any one or more of the promoters, enhancers and/or other sequences described in U.S. Pat. No. 8,708,948B2, U.S. Pat. No. 9,1385,96B2, U.S. Pat. No. 10,286,085B2, and U.S. Pat. No. 8,538,520B2, the contents of each of which are incorporated herein by reference in their entireties.

(iii) Angelman Syndrome-Associated Gene

As used herein, an “Angelman syndrome-associated gene” refers to any gene in a subject with Angelman syndrome which can be targeted by gene therapy to alleviate at least one symptom of Angelman syndrome. In some embodiments, the level of the protein encoded by the Angelman syndrome-associated gene is reduced or undetectable in subjects with Angelman syndrome. In some embodiments, the Angelman syndrome-associated gene encodes a protein that contributes to normal neuron function.

In some embodiments, one or more mutations in the Angelman syndrome-associated gene (e.g., UBE3A gene) is present in subjects with Angelman syndrome. In some embodiments, loss of function of the Angelman syndrome-associated gene (e.g., UBE3A gene) is present in subjects with Angelman syndrome. In some embodiments, one or more mutations in the Angelman syndrome-associated gene; or reduced or loss of expression or function of the Angelman syndrome-associated gene, is associated with, promotes or causes Angelman syndrome. In some embodiments, mutations in the Angelman syndrome-associated gene results in the maternal copy of UBE3A gene being absent or not functioning normally.

The type of mutation in the Angelman syndrome-associated gene (e.g., UBE3A gene) is not limited, and may be an insertion, deletion, duplication and/or substitution. In some embodiments, the mutation in the UBE3A gene is associated with, promoted by, or caused by, uniparental disomy. In some embodiments, the mutation in the UBE3A gene is associated with, promoted by, or caused by, an imprinting defect. In some embodiments, the mutation in the UBE3A gene is associated with, promoted by, or caused by, one or more translocations. In some embodiments, the mutation in the UBE3A gene is any UBE3A mutation that has been identified in patients with Angelman syndrome. For instance, the mutation in the UBE3A gene is selected from one or more UBE3A gene mutations described in Dagli A I, et al. Angelman Syndrome. 1998 Sep. 15 GeneReviews, which is incorporated herein by reference in its entirety for all purposes.

The disclosure provides AAV expression cassettes comprising an Angelman syndrome-associated gene. In some embodiments, an AAV expression cassette comprises an Angelman syndrome-associated gene which encodes a protein, including therapeutic (e.g., for medical or veterinary uses) or immunogenic (e.g., for vaccines) polypeptide. In some embodiments, the AAV expression cassette comprises a mammalian Angelman syndrome-associated gene. In some embodiments, the AAV expression cassette comprises a human Angelman syndrome-associated gene. In some embodiments, the AAV expression cassette comprises an Angelman syndrome-associated gene that encodes ubiquitin protein ligase E3A (UBE3A).

In some embodiments, the transgene encodes a human UBE3A. In some embodiments, the human UBE3A comprises the amino acid sequence with at least 70% identity (for example, at least 75%, at least 80%, at least 85%, at least 90%, at least 95%, at least 96%, at least 97%, at least 98%, at least 99%, at least 99.5%, or 100% identity, inclusive of all values and subranges that lie therebetween) to SEQ ID NO: 19.

In some embodiments, the Angelman syndrome-associated transgene comprises a nucleic acid sequence having at least 70% identity (for example, at least 75%, at least 80%, at least 85%, at least 90%, at least 95%, at least 96%, at least 97%, at least 98%, at least 99%, at least 99.5%, or 100% identity, inclusive of all values and subranges that lie therebetween) to SEQ ID NO: 12. In some embodiments, the Angelman syndrome-associated transgene comprises a nucleic acid sequence having at least 90% identity of SEQ ID NO: 12.

In some embodiments, the Angelman syndrome-associated transgene comprises a mutation capable of removing a predicted cryptic splice site. In some embodiments, the Angelman syndrome-associated transgene comprises a nucleic acid substitution of G2556C, relative to the nucleic acid sequence of wild type human UBE3A. In some embodiments, the Angelman syndrome-associated transgene comprises a nucleic acid substitution of G2556C, relative to the nucleic acid sequence of SEQ ID NO: 12. In some embodiments, the Angelman syndrome-associated transgene comprises a nucleic acid sequence having at least 90% identity of SEQ ID NO: 12, and a nucleic acid substitution of G2556C, relative to SEQ ID NO: 12.

In some embodiments, the human UBE3A comprises the nucleic acid sequence with at least 70% identity (for example, at least 75%, at least 80%, at least 85%, at least 90%, at least 95%, at least 96%, at least 97%, at least 98%, at least 99%, at least 99.5%, or 100% identity, inclusive of all values and subranges that lie therebetween) to SEQ ID NO: 5. In some embodiments, the Angelman syndrome-associated transgene comprises a nucleic acid sequence having at least 90% identity of SEQ ID NO: 5.

In some embodiments, the AAV expression cassette comprises a Kozak sequence. The Kozak sequence is a nucleic acid sequence that functions as a protein translation initiation site in many eukaryotic mRNA transcripts. In some embodiments, the Kozak sequence overlaps with the start codon. In some embodiments, the Kozak sequence comprises a nucleic acid sequence having at least 70% identity (for example, at least 75%, at least 80%, at least 85%, at least 90%, at least 95%, at least 96%, at least 97%, at least 98%, at least 99%, at least 99.5%, or 100% identity, inclusive of all values and subranges that lie therebetween) to the nucleic acid sequence of SEQ ID NO: 14 or acagccacc. In some embodiments, the Kozak sequence comprises a nucleic acid sequence of SEQ ID NO: 14, or a sequence at least 90% identical thereto; or a nucleic acid sequence of acagccacc, or a sequence at least 90% identical thereto.

### (iv) Polyadenylation (PolyA) Signal

Polyadenylation signals are nucleotide sequences found in nearly all mammalian genes and control the addition of a string of approximately 200 adenosine residues (the poly(A) tail) to the 3′ end of the gene transcript. The poly(A) tail contributes to mRNA stability, and mRNAs lacking the poly(A) tail are rapidly degraded. There is also evidence that the presence of the poly(A) tail positively contributes to the translatability of mRNA by affecting the initiation of translation.

In some embodiments, the AAV expression cassettes of the disclosure comprise a polyadenylation signal. The polyadenylation signal may be selected from the polyadenylation signal of simian virus 40 (SV40), rabbit beta globin (rBG), α-globin, β-globin, human collagen, human growth hormone (hGH), polyoma virus, human growth hormone (hGH) and bovine growth hormone (bGH).

In some embodiments, the AAV expression cassette comprises a bGH polyadenylation signal. In some embodiments, the bGH polyadenylation signal comprises a nucleic acid sequence having at least 70% identity (for example, at least 75%, at least 80%, at least 85%, at least 90%, at least 95%, at least 96%, at least 97%, at least 98%, at least 99%, at least 99.5%, or 100% identity, inclusive of all values and subranges that lie therebetween) to the nucleic acid sequence of SEQ ID NO: 6. In some embodiments, the bGH polyadenylation signal comprises a nucleic acid sequence of SEQ ID NO: 6, or a sequence at least 90% identical thereto.

In some embodiments, the polyadenylation signal is the SV40 polyadenylation signal. In some embodiments, the polyadenylation signal is the rBG polyadenylation signal. In some embodiments, the polyadenylation signal comprises the sequence of SEQ ID NO: 20 or SEQ ID NO: 21. In some embodiments, the polyadenylation signal comprises a sequence at least 90%, at least 95%, at least 96%, at least 97%, at least 98%, or at least 99% identical to the sequence of SEQ ID NO: 20 or SEQ ID NO: 21.

### (v) Stuffer Sequences

AAV vectors typically accept inserts of DNA having a defined size range which is generally about 4 kb to about 5.2 kb, or slightly more. Thus, for shorter sequences, it may be necessary to include additional nucleic acid in the insert fragment to achieve the required length which is acceptable for the AAV vector. Accordingly, in some embodiments, the AAV expression cassettes of the disclosure may comprise a stuffer sequence. The stuffer sequence may be for example, a sequence between 1-10, 10-20, 20-30, 30-40, 40-50, 50-60, 60-75, 75-100, 100-150, 150-200, 200-250, 250-300, 300-400, 400-500, 500-750, 750-1,000, 1,000-1,500, 1,500-2,000, 2,000-2,500, 2,500-3,000, 3,000-3,500, 3,500-4,000, 4,000-4,500, or 4,500-5,000, or more nucleotides in length. The stuffer sequence can be located in the cassette at any desired position such that it does not prevent a function or activity of the vector.

In some embodiments, the AAV cassette comprises at least one stuffer sequence. In some embodiments, the stuffer sequence comprises a nucleic acid sequence having at least 70% identity (for example, at least 75%, at least 80%, at least 85%, at least 90%, at least 95%, at least 96%, at least 97%, at least 98%, at least 99%, at least 99.5%, or 100% identity, inclusive of all values and subranges that lie therebetween) to the nucleic acid sequence of SEQ ID NO: 7. In some embodiments, the stuffer sequence comprises a nucleic acid sequence of SEQ ID NO: 7, or a sequence at least 90% identical thereto. In some embodiments, the stuffer sequence comprises a nucleic acid sequence of SEQ ID NO: 7, or a portion thereof. In some embodiments, the stuffer sequence comprises a portion (e.g., a 500-nucleotide long portion) of the nucleic acid sequence of SEQ ID NO: 7, or a sequence at least 90% identical thereto.

### (vi) Intronic Sequences

In some embodiments, the AAV expression cassettes of the disclosure may comprise an intronic sequence. In some embodiments, inclusion of an intronic sequence enhances expression compared with expression in the absence of the intronic sequence.

In some embodiments, the intronic sequence is a hybrid or chimeric sequence. In some embodiments, the intronic sequence is isolated or derived from an intronic sequence of one or more of SV40 (SV40IN), β-globin, chicken beta-actin, minute virus of mice (MVM), factor IX, and/or human IgG (heavy or light chain). In some embodiments, the intronic sequence is chimeric.

In some embodiments, the intron is derived from the human β-globin gene (hBGIN). In some embodiments, the intron comprises one or more of the following mutations: (i) mutation at the 5′ terminus to contain Exon 2 splicing donor (AGG), (ii) mutation at the 3′ terminus to contain Exon 3 splicing acceptor (CTC), and (iii) G74T and G205A, relative to SEQ ID NO: 13. In some embodiments, the intron comprises a mutation at the 5′ terminus to contain Exon 2 splicing donor (AGG). In some embodiments, the intron comprises a mutation at the 3′ terminus to contain Exon 3 splicing acceptor (CTC). In some embodiments, the intron comprises the mutation G74T and/or G205A, relative to SEQ ID NO: 13. In some embodiments, the intron comprises the following mutations: (i) mutation at the 5′ terminus to contain Exon 2 splicing donor (AGG), (ii) mutation at the 3′ terminus to contain Exon 3 splicing acceptor (CTC), and (iii) G74T and G205A, relative to SEQ ID NO: 13.

In some embodiments, the intronic sequence comprises a nucleic acid sequence having at least 70% identity (for example, at least 75%, at least 80%, at least 85%, at least 90%, at least 95%, at least 96%, at least 97%, at least 98%, at least 99%, at least 99.5%, or 100% identity, inclusive of all values and subranges that lie therebetween) to the nucleic acid sequence of SEQ ID NO: 4. In some embodiments, the intronic sequence comprises the sequence of SEQ ID NO: 4, or a sequence at least 90% identical thereto. In some embodiments, the intronic sequence comprises the sequence of SEQ ID NO: 4.

### AAV Production Methods

The AAV expression cassettes described herein may be incorporated into a vector (e.g., a plasmid or a bacmid) using standard molecular biology techniques. The disclosure provides vectors comprising any one of the AAV expression cassettes described herein. The vector (e.g., plasmid or bacmid) may further comprise one or more genetic elements used during production of AAV, including, for example, AAV rep and cap genes, and helper virus protein sequences.

The AAV expression cassettes, and vectors (e.g., plasmids) comprising the AAV expression cassettes described herein may be used to produce recombinant AAV vectors.

The disclosure provides methods for producing a recombinant AAV vector comprising contacting an AAV producer cell (e.g., an HEK293 cell) with an AAV expression cassette, or vector (e.g., plasmid) of the disclosure. The disclosure further provides cells comprising any one of the AAV expression cassettes, or vectors disclosed herein. In some embodiments, the method further comprises contacting the AAV producer cell with one or more additional plasmids encoding, for example, AAV rep and cap genes, and helper virus protein sequences. In some embodiments, a method for producing a recombinant AAV vector comprises contacting an AAV producer cell (e.g., an insect cell such as a Sf9 cell) with at least one insect cell-compatible vector comprising an AAV expression cassette of the disclosure. An “insect cell-compatible vector” is any compound or formulation (biological or chemical), which facilitates transformation or transfection of an insect cell with a nucleic acid. In some embodiments, the insect cell-compatible vector is a baculoviral vector. In some embodiments, the method further comprises maintaining the insect cell under conditions such that AAV is produced.

The disclosure provides recombinant AAV vectors produced using any one of the methods disclosed herein. The recombinant AAV vectors produced may be of any serotype, for example AAV1, AAV2, AAV3, AAV4, AAV5, AAV6, AAV7, AAV8, AAV9, AAV10, AAV11, AAV12, AAVrh8, AAVrh10, AAVrh32.33, AAVrh74, Avian AAV or Bovine AAV. In some embodiments, the recombinant AAV vectors produced may comprise one or more AAV capsid protein having one or more amino acid modifications (e.g., substitutions and/or deletions) compared to the native AAV capsid. For example, the recombinant AAV vectors may be modified AAV vectors derived from AAV1, AAV2, AAV3, AAV4, AAV5, AAV6, AAV7, AAV8, AAV9, AAV10, AAV11, AAV12, AAVrh8, AAVrh10, AAVrh32.33, AAVrh74, Avian AAV and Bovine AAV. In some embodiments, the recombinant AAV vector is a single-stranded AAV (ssAAV). In some embodiments, the recombinant AAV vector is a self-complementary AAV (scAAV).

In some embodiments, the AAV vector comprises a capsid protein of AAV1, AAV2, AAV3, AAV4, AAV5, AAV6, AAV7, AAV8, AAV9, AAV10, AAV11, AAV12, AAVrh8, AAVrh10, AAVrh32.33, AAVrh74, Avian AAV or Bovine AAV. In some embodiments, the AAV vector comprises a capsid protein with one or more substitutions or mutations, as compared to a wild type AAV capsid protein. The recombinant AAV vectors disclosed herein may be used to transduce target cells with the transgene sequence, for example by contacting the recombinant AAV vector with a target cell.

In some embodiments, the AAV vector comprises a capsid protein comprising: an amino acid sequence with at least 70% identity (for example, at least 75%, at least 80%, at least 85%, at least 90%, at least 95%, at least 96%, at least 97%, at least 98%, at least 99%, at least 99.5%, or 100% identity, inclusive of all values and subranges that lie therebetween) to SEQ ID NO: 15. In some embodiments, In some embodiments, the AAV vector comprises a capsid protein comprising the amino acid sequence of SEQ ID NO: 15, or a sequence at least 90% identical thereto. In some embodiments, the AAV vector comprises a capsid protein comprising the amino acid sequence of SEQ ID NO: 15.

In some embodiments, the AAV vector comprises a capsid protein comprising: an amino acid sequence with at least 70% identity (for example, at least 75%, at least 80%, at least 85%, at least 90%, at least 95%, at least 96%, at least 97%, at least 98%, at least 99%, at least 99.5%, or 100% identity, inclusive of all values and subranges that lie therebetween) to SEQ ID NO: 16. In some embodiments, the AAV vector comprises a capsid protein comprising the amino acid sequence of SEQ ID NO: 16, or a sequence at least 90% identical thereto. In some embodiments, the AAV vector comprises a capsid protein comprising the amino acid sequence of SEQ ID NO: 16.

In some embodiments, the AAV vector comprises a capsid protein comprising: an amino acid sequence with at least 70% identity (for example, at least 75%, at least 80%, at least 85%, at least 90%, at least 95%, at least 96%, at least 97%, at least 98%, at least 99%, at least 99.5%, or 100% identity, inclusive of all values and subranges that lie therebetween) to SEQ ID NO: 17. In some embodiments, the AAV vector comprises a capsid protein comprising the amino acid sequence of SEQ ID NO: 17, or a sequence at least 90% identical thereto. In some embodiments, the AAV vector comprises a capsid protein comprising the amino acid sequence of SEQ ID NO: 17.

In some embodiments, the AAV vector comprises a capsid protein comprising: (i) the amino acid sequence of SEQ ID NO: 15, or a sequence at least 90% identical thereto, or (ii) the amino acid sequence of SEQ ID NO: 16, or a sequence at least 90% identical thereto, or (iii) the amino acid sequence of SEQ ID NO: 17, or a sequence at least 90% identical thereto.

### Methods of Expression and Treatment

The disclosure provides compositions comprising any one of the nucleic acids, AAV expression cassettes, plasmids, cells, or recombinant AAV vectors disclosed herein. In some embodiments, the compositions disclosed herein comprise at least one pharmaceutically acceptable carrier, excipient, and/or vehicle, for example, solvents, buffers, solutions, dispersion media, coatings, antibacterial and antifungal agents, isotonic and absorption delaying agents. In some embodiments, the pharmaceutically acceptable carrier, excipient, and/or vehicle may comprise saline, buffered saline, dextrose, water, glycerol, sterile isotonic aqueous buffer, and combinations thereof. In some embodiments, the pharmaceutically acceptable carrier, excipient, and/or vehicle comprises phosphate buffered saline, sterile saline, lactose, sucrose, calcium phosphate, dextran, agar, pectin, peanut oil, sesame oil, pharmaceutical grades of mannitol, lactose, starch, magnesium stearate, sodium saccharine, cellulose, magnesium carbonate, polyol (e.g., glycerol, propylene glycol, and liquid polyethylene glycol, and the like) or suitable mixtures thereof. In some embodiments, the compositions disclosed herein further comprise minor amounts of emulsifying or wetting agents, or pH buffering agents.

In some embodiments, the compositions disclosed herein further comprise other conventional pharmaceutical ingredients, such as preservatives, or chemical stabilizers, such as chlorobutanol, potassium sorbate, sorbic acid, sulfur dioxide, propyl gallate, the parabens, ethyl vanillin, glycerin, phenol, parachlorophenol or albumin. In some embodiments, the compositions disclosed herein may further comprise antibacterial and antifungal agents, such as, parabens, chlorobutanol, phenol, sorbic acid or thimerosal; isotonic agents, such as, sugars or sodium chloride and/or agents delaying absorption, such as, aluminum monostearate and gelatin.

The disclosure provides methods of expressing an Angelman syndrome-associated transgene in a cell, comprising: contacting the cell with any one of the nucleic acid molecules, plasmids, cells, recombinant AAV vectors, or compositions disclosed herein, thereby expressing the Angelman syndrome-associated transgene in the cell.

The disclosure provides methods of expressing an Angelman syndrome-associated transgene in a tissue, comprising: contacting the tissue with any one of the nucleic acid molecules, plasmids, cells, recombinant AAV vectors, or compositions disclosed herein, thereby expressing the Angelman syndrome-associated transgene in the tissue. In some embodiments, the tissue comprises at least one cell.

In some embodiments, the cell is a neuronal cell. In some embodiments, the cell is a dividing cell, such as a cultured cell in cell culture. In some embodiments, the cell is a non-dividing cell. In some embodiments, the Angelman syndrome-associated gene is delivered to the cell in vitro, e.g., to produce the Angelman syndrome-associated polypeptide in vitro or for ex vivo gene therapy.

In some embodiments, the contacting step is performed in vitro, ex vivo, or in vivo. In some embodiments, the contacting step is performed in vivo in a subject in need thereof. In some embodiments, the contacting step comprises administering a therapeutically effective amount of the nucleic acid molecule, the plasmid, the recombinant AAV vector, or the composition to the subject. In some embodiments, the subject suffers from, or is at a risk of developing the Angelman syndrome.

The disclosure provides methods for treating Angelman syndrome in a subject in need thereof, comprising administering to the subject a therapeutically effective amount of any one of the nucleic acid molecules, plasmids, cells, recombinant AAV vectors, or compositions disclosed herein, thereby treating Angelman syndrome in the subject. In some embodiments, the subject suffers from, or is at a risk of developing the Angelman syndrome. In some embodiments, the Angelman syndrome is associated with, promoted by, or caused by a genetic change. In some embodiments, the genetic change comprises one or more genetic changes (for example, one or more deletions, insertions, duplications and/or substitutions) to the UBE3A gene, as compared to the wild type UBE3A gene, and/or alterations to the expression and/or activity of the UBE3A protein, as compared with a wild type UBE3A protein. In some embodiments, the subject at a risk of developing Angelman syndrome is a newborn who is identified as carrying a mutation in the UBE3A gene. In some embodiments, the Angelman syndrome-associated gene (e.g., UBE3A) is targeted by gene therapy to increase its expression and/or function.

In some embodiments, the method comprises diminishing the severity of; delaying the onset or progression of; and/or eliminating a symptom of the Angelman syndrome. In some embodiments, the symptom of the Angelman syndrome comprises: (a) developmental delay, (b) intellectual disability, (c) speech impairment, (d) gait ataxia, (e) tremulousness of the limbs, (f) frequent laughing or smiling, (g) excitability, (h) microcephaly, (i) seizures, (j) trouble sleeping, (k) tongue thrusting, (l) hand flapping, (m) curved spine or (n) any combination thereof.

In some embodiments, the methods comprise prolonging the survival of the subject, as compared to a control subject having Angelman syndrome, wherein the control subject has not been administered the therapeutically effective amount. In some embodiments, the methods comprise prolonging the survival of the subject, as compared to the expected survival of the subject prior to administration of the therapeutically effective amount. In some embodiments, the methods comprise prolonging the survival of the subject by a value in the range of about 3 months to about 50 years (for example, about 6 months, about 1 year, about 5 years, about 10 years, about 15 years, about 20 years, about 25 years, about 30 years, about 35 years, about 40 years, about 45 years, about 50 years, including the subranges and values that lie therebetween), as compared to: (i) a control subject having Angelman syndrome, wherein the control subject has not been administered the therapeutically effective amount, or (ii) the expected survival of the subject prior to administration of the therapeutically effective amount.

Dosages of the recombinant AAV vector to be administered to a subject depend upon the mode of administration, the disease or condition to be treated and/or prevented, the individual subject's condition, the particular virus vector or capsid, the nucleic acid to be delivered, and the like, and can be determined in a routine manner. Exemplary doses for achieving therapeutic effects are titers of at least about 10⁵, about 10⁶, about 10⁷, about 10⁸, about 10⁹, about 10¹⁰, about 10¹¹, about 10¹², about 10¹³, about 10¹⁴, about 10¹⁵ transducing units, optionally about 10⁸ to about 10¹³ transducing units.

In particular embodiments, more than one administration (e.g., two, three, four or more administrations) may be employed to achieve the desired level of gene expression over a period of various intervals, e.g., daily, weekly, monthly, yearly, etc.

In some embodiments, the subject is a human subject. Exemplary modes of administration include oral, transmucosal, intrathecal, transdermal, parenteral (e.g., intravenous, subcutaneous, intradermal, intramuscular [including administration to skeletal, diaphragm and/or cardiac muscle], intradermal, intrapleural, intracerebral, and intraarticular), intracerebroventricular (ICV) injection (e.g. bilateral ICV injection), intralymphatic, and the like, as well as direct tissue or organ injection (e.g., to liver, skeletal muscle, cardiac muscle, diaphragm muscle, or brain). Delivery to a target tissue can also be achieved by delivering a depot comprising the virus vector and/or capsid.

In some embodiments, the methods disclosed herein may comprise administering to the subject a therapeutically effective amount of any one of the nucleic acids, AAV expression cassettes, plasmids, cells, recombinant AAV vectors, or compositions disclosed herein in combination with one or more secondary therapies targeting Angelman syndrome. In some embodiments, the methods of treating and/or delaying the onset of at least one symptom of Angelman syndrome in a subject disclosed herein may further comprise administering one or more secondary therapies targeting Angelman syndrome. The term administered “in combination,” as used herein, is understood to mean that two (or more) different treatments are delivered to the subject during the course of the subject's affliction with the disorder (e.g., Angelman syndrome), such that the effects of the treatments on the patient overlap at a point in time. In certain embodiments, the delivery of one treatment is still occurring when the delivery of the second begins, so that there is overlap in terms of administration. This is sometimes referred to herein as “simultaneous” or “concurrent” delivery. In other embodiments, the delivery of one treatment ends before the delivery of the other treatment begins, which may be referred to as “sequential” delivery.

In some embodiments, the treatment is more effective because of combined administration. For example, the second treatment is more effective, an equivalent effect is seen with less of the second treatment, or the second treatment reduces symptoms to a greater extent than would be seen if the second treatment were administered in the absence of the first treatment, or the analogous situation is seen with the first treatment. The effect of the two treatments can be partially additive, wholly additive, or greater than additive (synergistic).

All papers, publications and patents cited in this specification are herein incorporated by reference as if each individual paper, publication, or patent were specifically and individually indicated to be incorporated by reference and are incorporated herein by reference to disclose and describe the methods and/or materials in connection with which the publications are cited. However, mention of any reference, article, publication, patent, patent publication, and patent application cited herein is not, and should not be taken as an acknowledgment or any form of suggestion that they constitute valid prior art or form part of the common general knowledge in any country in the world.

Unless the context indicates otherwise, it is specifically intended that the various features described herein can be used in any combination.

Unless otherwise defined, all technical and scientific terms used herein have the same meaning as commonly understood by one of ordinary skill in the art to which this disclosure belongs.

It is to be understood that the description above as well as the examples that follow are intended to illustrate, and not limit, the scope of the invention. Other aspects, advantages, and modifications within the scope of the invention will be apparent to those skilled in the art to which the invention pertains.

### EXAMPLES

The following examples, which are included herein for illustration purposes only, are not intended to be limiting.

### Example 1: Design of Adeno-Associated Virus (AAV) Cassettes Encoding Human UBE3A

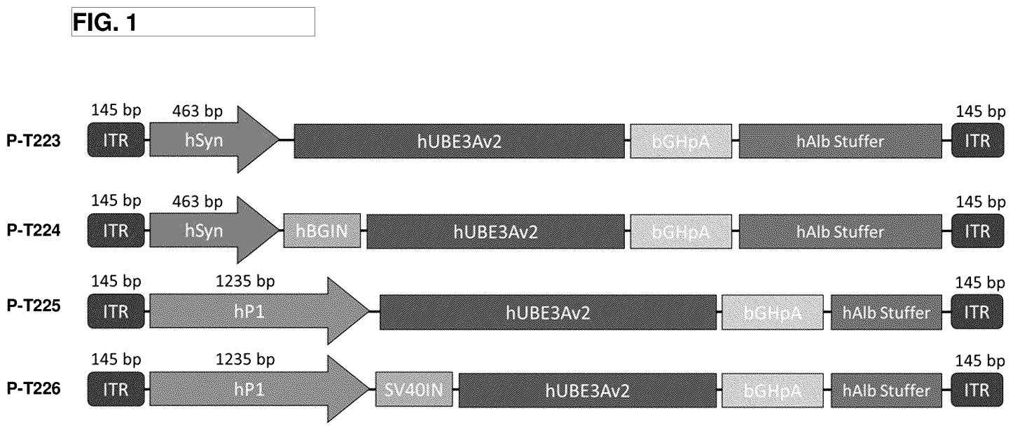

Several AAV cassettes comprising elements in various orders and combinations were generated to test the expression of a human ubiquitin protein ligase E3A (hUBE3A) gene and the production of functional UBE3A protein (FIG. 1). Each of the cassettes shown in FIG. 1 comprises a hUBE3A gene, comprising the nucleic acid substitution of G2556C; this mutant hUBE3A gene is referred to herein as hUBE3Av2 and comprises the nucleic acid sequence of SEQ ID NO: 5. Without being bound by a theory, it is thought that the nucleic acid substitution of G2556C acts as a silent mutation to remove a strongly predicted cryptic splice site.

Each of the cassettes also comprises one or more stuffer sequences, such as a human albumin (hAlb) stuffer sequence, and/or an intron (such as, a human β-globin intron (hBGIN) or a SV40 intron), inserted upstream and/or downstream of the hUBE3Av2 gene, as indicated in FIG. 1. Without being bound by a theory, it is thought that the inclusion of one or more stuffer sequences might enhance transgene expression. Each of the cassettes also comprises a bovine growth hormone polyA signal (bGHpA; SEQ ID NO: 6), a 5′ internal terminal repeat (ITR; SEQ ID NO: 2), and a 3′ ITR (SEQ ID NO: 8). The expression of hUBE3Av2 is driven by a human Synapsin (hSyn) promoter (SEQ ID NO: 3; used in cassettes P-T223 and P-T224), or the human putative endogenous promoter 1 (hP1 promoter comprising the sequence of SEQ ID NO: 24; e.g., in cassettes P-T225 and P-T226). Without being bound by a theory, it is thought that the hSyn promoter might drive tissue-specific expression of the gene, for example, in the brain. Each cassette contains a packaged genome of about 4.7 kilobases (kB).

The hBGIN used in these cassettes (comprising the nucleic acid sequence of SEQ ID NO: 4) was mutated at the 5′- and 3′-termini to contain the hBGIN Exon 2 splicing donor (AGG) and hBGIN Exon 3 splicing acceptor (CTC), respectively. Without being bound by a theory, it is thought that these mutations in the hBGIN might enable efficient splicing. Additionally, hBGIN was mutated at G74T and G205A to remove a strongly-predicted splice acceptor site. Without being bound by a theory, it is thought that the G205A mutation in the hBGIN might prevent premature splicing.

### Example 2: Expression of AAV Cassettes Encoding Human UBE3A in Induced Pluripotent Stem Cells (iPSCs)

The following AAV cassettes: P-T116, P-T178, P-T223, P-T224, P-T225, and P-T226 were packaged into AAV particles, which were then used to transduce iPSCs. The transduced iPSCs were transduced, lysed, and analyzed for mRNA expression by RT-qPCR to test the expression of hUBE3Av2. The arrangement of the elements in the P-T116 cassette is: pTR141-hP1-SV40IN-hUBE3Av1-SV40 pA, and the P-T116 cassette comprises the nucleic acid sequence of SEQ ID NO: 22. The arrangement of the elements in the P-T178 cassette is: pTR141-hSyn-SV40IN-hUBE3Av1-SV40 pA. P-T116 and P-T178 vary only in the promoter, and the P-T178 cassette comprises the nucleic acid sequence of SEQ ID NO: 23.

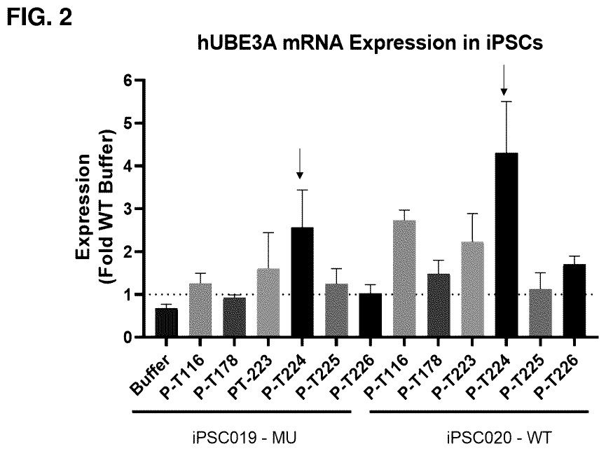

To measure gene expression, each cassette was transduced into both WT iPSCs and mutant (MU) UBE3^{-/+} iPSCs and the expression of hUBE3A mRNA was measured by RT-qPCR (FIG. 2). Of the six cassettes tested, the highest levels of hUBE3A mRNA were seen to be expressed from the cassette P-T224 (comprising the nucleic acid sequence of SEQ ID NO: 1) in both WT and MU iPSCs. These results indicate that the elements present in the P-T224 cassette, and the specific order and combination thereof, promotes efficient expression of the hUBE3A transgene from P-T224. These results also suggest that the inclusion of the mutated hBGIN disclosed herein results in higher expression of hUBE3A mRNA from the P-T224 cassette, as P-T224 is the only cassette with this sequence.

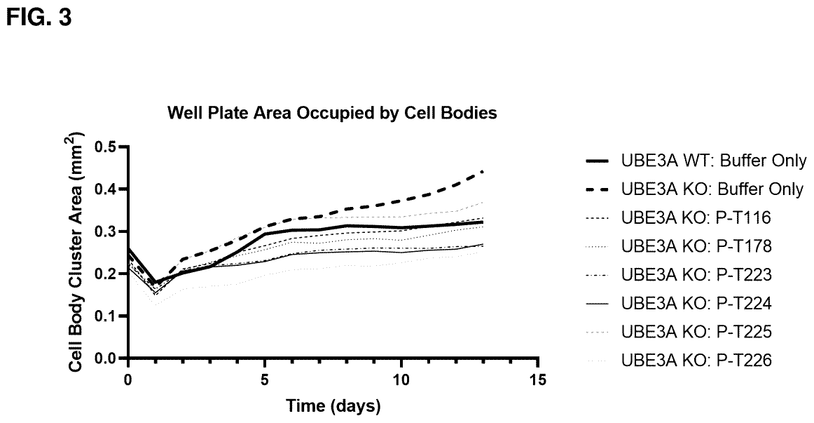

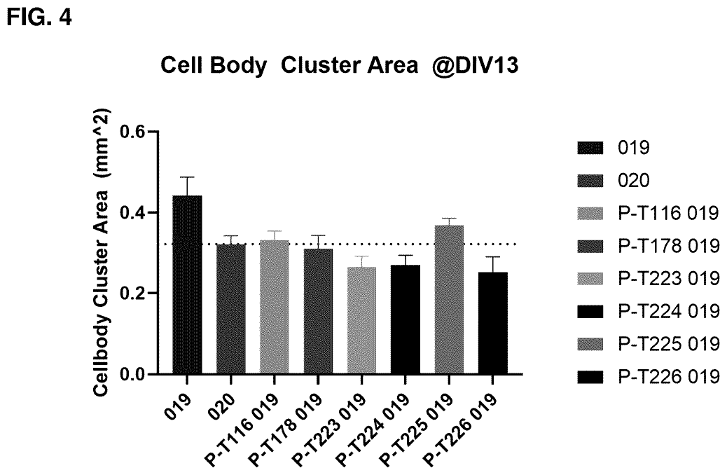

To evaluate whether the expression of the AAV cassettes encoding hUBE3A is capable of rescuing the phenotype of cells lacking hUBE3A function, each cassette was transduced into MU UBE3^{-/+} iPSCs and the cell body cluster areas (mm²) were measured (FIG. 3 and FIG. 4). The cell body cluster area is a measurement of the area in a well that is occupied by cell bodies (as opposed to cell bodies and neurites). Without being bound by a theory, it is thought that measurement of this marker indicates the rescue of UBE3A function in UBE3^{-/+} iPSCs by the transduced AAVs.

The cell body cluster areas were compared to WT or MU UBE3^{-/+} iPSCs and were measured across 13 days (FIG. 3). FIG. 4 shows that by day 13, the transduction of P-T223, P-T224, and P-T226 cassettes provided the lowest cell body cluster area, as compared to (i) the transduction of the other three cassettes, (ii) WT iPSCs or (iii) MU UBE3^{-/+} iPSCs (FIG. 4).

In sum, the results described above demonstrate the successful expression of hUBE3A mRNA from all six cassettes, while demonstrating that the expression of UBE3Av2 was highest from the cassette P-T224, as compared to the other five cassettes (P-T116, P-T178, P-T223, P-T225, and P-T226). The results also show that AAV-mediated expression of hUBE3A results in rescuing the loss of UBE3A function, as seen by the reduction in the cell body cluster area.

Overall, the results indicate that the elements present in the P-T224 cassette, and the specific order and combination thereof, promotes efficient expression of the hUBE3A transgene from P-T224. Moreover, these results demonstrate that AAV-mediated expression of the mutated version of hUBE3A disclosed herein using the expression cassette elements disclosed herein is capable of rescuing the phenotype of cells lacking UBE3A function.

### Example 3: Administration of AAV Vectors Comprising P-T224 Restores Wild-Type UBE3A Protein Levels in Brains of UBE3^{-/+} Mice

P-T223, P-T224, P-T225, and P-T226 cassettes were tested for expression of UBE3A mRNA and UBE3A protein in mice. Each of the AAV cassettes was packaged within an AAV capsid, comprising the AAV capsid protein (SEQ ID NO: 16), and then the resulting AAV particles were administered into P1 neonatal mice with a UBE3^{-/+} genotype, while a control vehicle was administered to mice with either a wild-type (WT) or UBE3^{-/+} genotype (heterozygous, HET)—see Table A below. The AAV particles were administered by bilateral intracerebroventricular (ICV) injection on postnatal day 1 (PND1) at a dosage of 1.6×10¹¹ vg in 2 μL per bilateral ventricle (4 μL total; flow rate: 1 μL/min). Three weeks post-injection, mice were assessed by molecular analysis and histology across brain (anterior and posterior) and liver tissue samples.

TABLE A

| | | | Cassette | Selected Cassette | |
|---|---|---|---|---|---|
| Group | Genotype | N | Number | Elements | Dose (vg/animal) |
| 1 | WT | 4 | NA | Vehicle | NA |
| 2 | Ube3a^{-/+} | 5 | NA | Vehicle | NA |
| 3 | Ube3a^{-/+} | 6 | P-T223 | Syn | 1.6e11 |
| 4 | Ube3a^{-/+} | 4 | P-T224 | Syn-hBGIN | 1.6e11 |
| 5 | Ube3a^{-/+} | 7 | P-T225 | hP1 | 1.6e11 |
| 6 | Ube3a^{-/+} | 6 | P-T226 | hP1-SV40IN | 1.6e11 |

The Ube3a″mouse model is a partial knockout (that is, the paternal allele is not mutated). Without being bound by a theory, it is thought that, in the neurons, paternal imprinting and a mutated maternal allele results in complete UBE3A knockout; however, this does not occur in other tissues (e.g. liver), which have reduced but detectable UBE3A protein.

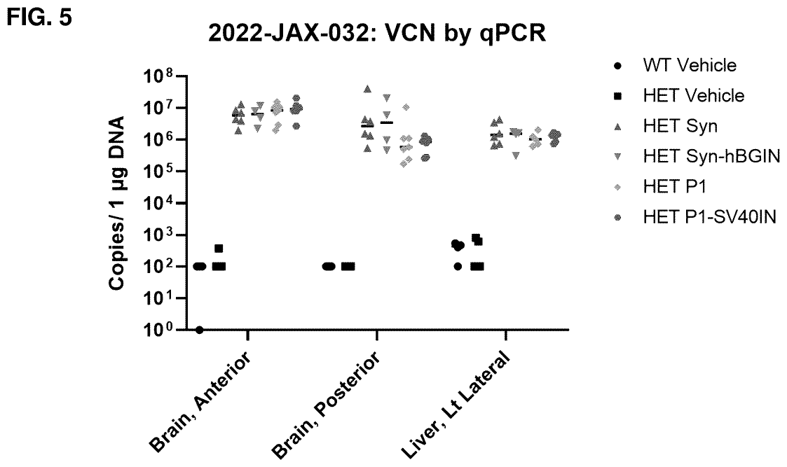

Administration of AAV particles comprising each of the AAV cassettes encoding UBE3A displayed high vector copy number (VCN) across the three tissue samples tested (anterior brain, posterior brain, and left lateral liver), as compared to the administration of vehicle control in WT and HET vehicle (FIG. 5). These results show that all the tested AAV particles are able to successfully transduce the tested tissues.

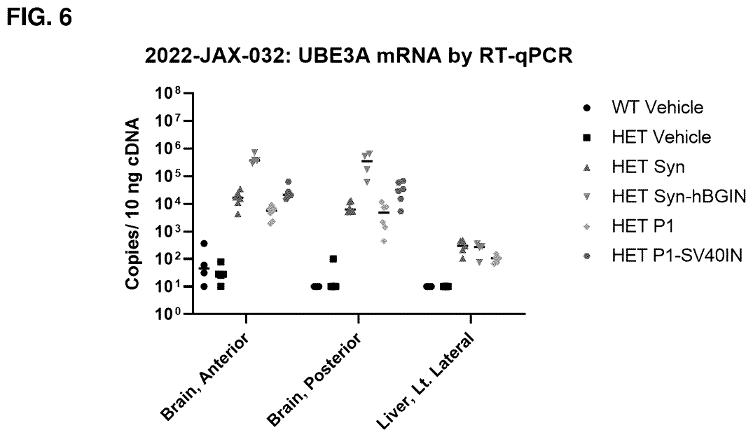

To evaluate the expression of UBE3A in these tissues, RT-qPCR was performed on the tissue samples to measure the levels of UBE3A mRNA. Surprisingly, even though AAV particles comprising each of the tested AAV cassettes transduced the tested tissues to similar levels, administration of AAV particles comprising cassette P-T224 (comprising hSyn and hBGIN; see FIG. 5 and Table A) resulted in higher levels of UBE3A mRNA expression in both brain anterior and brain posterior tissue samples as compared to P-T223, P-T225, and P-T226 (FIG. 6). Without being bound by a theory, it is thought that the inclusion of the hBGIN intron contributes to the about 1 log higher mRNA levels upon expression from the P-T224 cassette. Also, the expression of all the tested AAV cassettes was lower in the liver tissue, as compared to in the brain tissue, demonstrating the tissue specific expression of the cassettes.

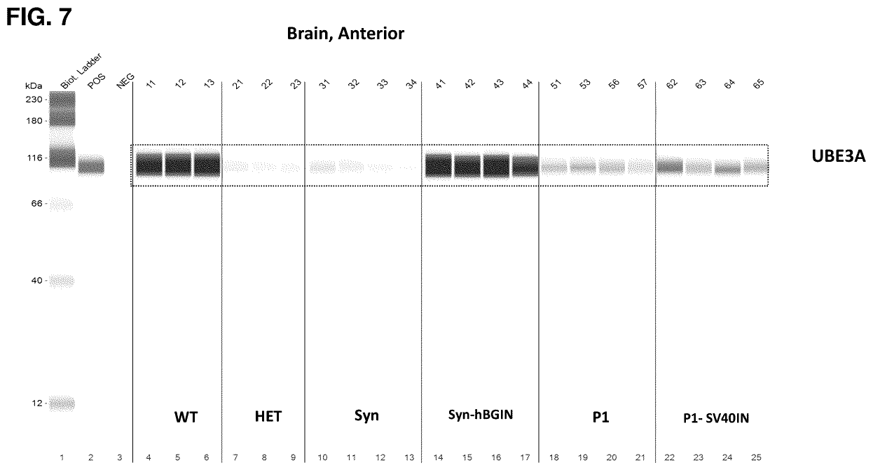

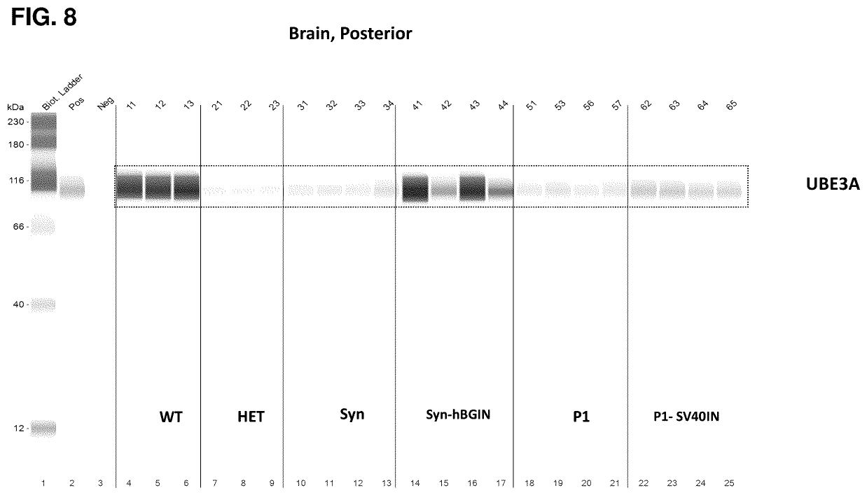

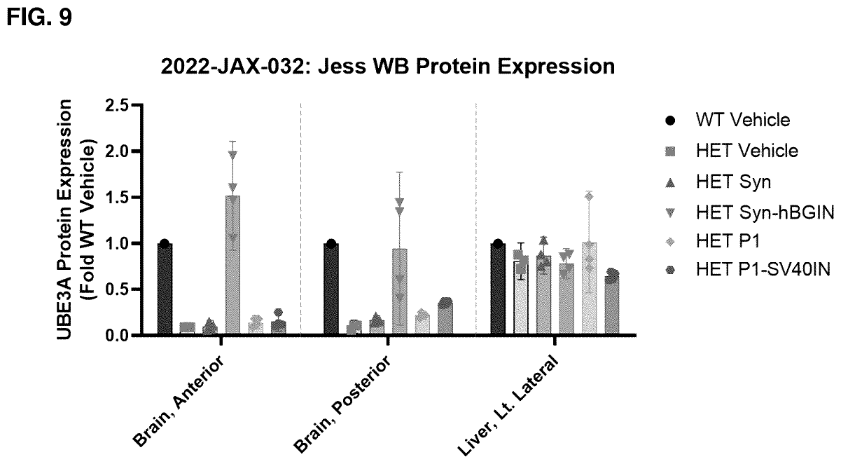

To evaluate the expression of UBE3A protein in these tissues, UBE3A protein levels were measured by Western blot analysis in brain anterior tissues (FIG. 7) and brain posterior tissues (FIG. 8). As shown in FIG. 7 and FIG. 8, the level of UBE3A protein expressed from AAV particles comprising P-T224 in brain tissue was comparable to UBE3A levels in wild type mice, indicating that the P-T224 cassette is able to drive effectively gene expression in neuronal cells of the brain tissue. Quantitation of the expressed UBE3A protein levels further confirmed that expression from the cassette P-T224 achieved restoration of UBE3A protein levels to near WT levels throughout both brain tissues (FIG. 9). Similar levels of UBE3A was seen to be expressed in the liver of all the different mice groups listed in Table A.

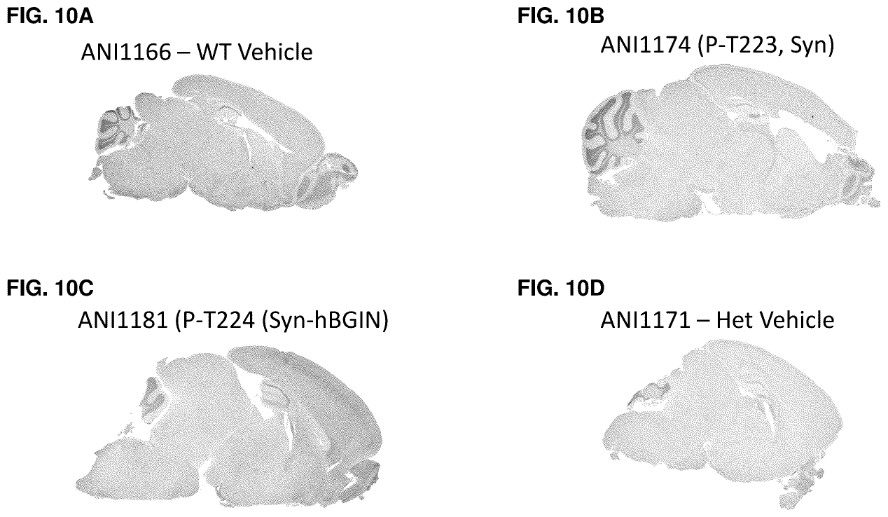

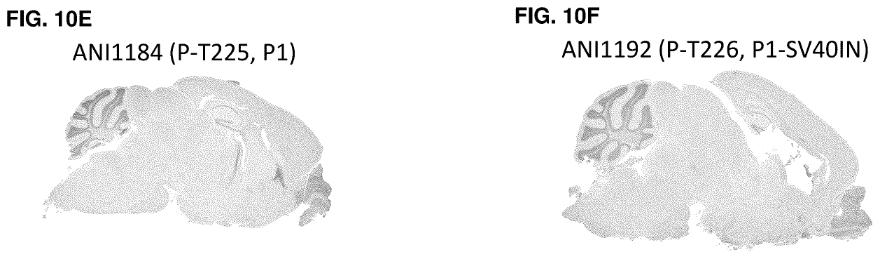

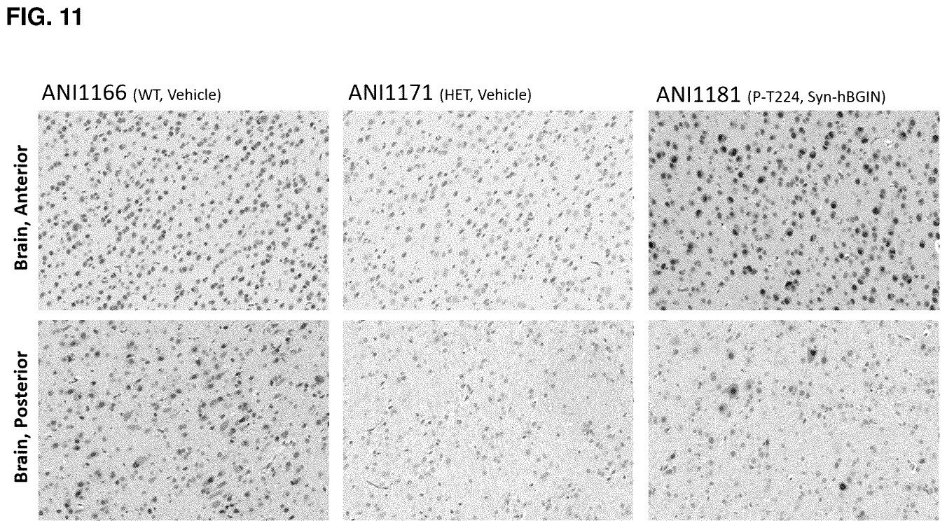

To further evaluate the expression and localization of the UBE3A protein in brain tissues, the following experiment was performed. Sagittal brain sections were analyzed with immunohistochemistry (IHC) using anti-hUBE3A antibody staining, which revealed that the AAV-mediated expression of UBE3A from the P-T224 cassette was able to produce detectable UBE3A protein in the both the anterior and posterior regions of the brain (FIG. 10A-FIG. 10F). UBE3A protein concentration was seen to be higher in the anterior brain regions as compared to the posterior brain regions (FIG. 11).

The results show that AAV-mediated expression of UBE3A from the P-T224 cassette results in superior UBE3A mRNA expression levels, UBE3A protein expression levels, and accurate localization of UBE3A in the target brain tissue in mice, as compared to the other AAV cassettes tested. Without being bound by a theory, it is thought that the unique combination of elements in the P-T224 cassette, such as, the hSyn promoter and the mutated hBGIN intronic sequence disclosed herein in combination with the mutated hUBE3Av2 gene disclosed herein contributes to the effective expression of the target gene from the P-T224 cassette, which can promote in successful rescue of one or more symptoms characteristic of the Angelman syndrome.

SEQUENCES

| SEQ | | |
|---|---|---|
| ID | | |
| NO | Description | SEQUENCE |
| 1 | pTR145K-hSyn- | TTGGCCACTCCCTCTCTGCGCGCTCGCTCGCTCACTGAGGCCGGGCGACCA |
| | hBGINv2- | AAGGTCGCCCGACGCCCGGGCTTTGCCCGGGCGGCCTCAGTGAGCGAGCGA |
| | hUBE3Av2-bGHpA- | GCGCGCAGAGAGGGAGTGGCCAACTCCATCACTAGGGGTTCCTTGCGTCGA |
| | hAlb | CAGGGCCCTGCGTATGAGTGCAAGTGGGTTTTAGGACCAGGATGAGGCGGG |
| | | GTGGGGGTGCCTACCTGACGACCGACCCCGACCCACTGGACAAGCACCCAA |
| | | CCCCCATTCCCCAAATTGCGCATCCCCTATCAGAGAGGGGGAGGGGAAACA |
| | | GGATGCGGCGAGGCGCGTGCGCACTGCCAGCTTCAGCACCGCGGACAGTGC |
| | | CTTCGCCCCCGCCTGGCGGCGCGCGCCACCGCCGCCTCAGCACTGAAGGCG |
| | | CGCTGACGTCACTCGCCGGTCCCCCGCAAACTCCCCTTCCCGGCCACCTTG |
| | | GTCGCGTCCGCGCCGCCGCCGGCCCAGCCGGACCGCACCACGCGAGGCGCG |
| | | AGATAGGGGGGCACGGGCGCGACCATCTGCGCTGCGGCGCCGGCGACTCAG |
| | | CGCTGCCTCAGTCTGCGGTGGGCAGCGGAGGAGTCGTGTCGTGCCTGAGAG |
| | | CGCAGAAGCTTAGGGTGAGTCTATGGGACCCTTGATGTTTTCTTTCCCCTT |
| | | CTTTTCTATGGTTAAGTTCATGTCATAGGAAGGGGATAAGTAACAGGGTAC |
| | | ACATATTGACCAAATCAGGGTAATTTTGCATTTGTAATTTTAAAAAATGCT |
| | | TTCTTCTTTTAATATACTTTTTTGTTTATCTTATTTCTAATACTTTCCCTA |
| | | ATCTCTTTCTTTCAAGGCAATAATGATACAATGTATCATGCCTCTTTGCAC |
| | | CATTCTAAAGAATAACAGTGATAATTTCTGGGTTAAGGCAATAGCAATATT |
| | | TCTGCATATAAATATTTCTGCATATAAATTGTAACTGATGTAAGAGGTTTC |
| | | ATATTGCTAATAGCAGCTACAATCCAGCTACCATTCTGCTTTTATTTTATG |
| | | GTTGGGATAAGGCTGGATTATTCTGAGTCCAAGCTAGGCCCTTTTGCTAAT |
| | | CATGTTCATACCTCTTATCTTCCTCCCACAGCTCCACCGGTCGCCACCATG |
| | | AAGCGAGCAGCTGCAAAGCATCTAATAGAACGCTACTACCACCAGTTAACT |
| | | GAGGGCTGTGGAAATGAAGCCTGCACGAATGAGTTTTGTGCTTCCTGTCCA |
| | | ACTTTTCTTCGTATGGATAATAATGCAGCAGCTATTAAAGCCCTCGAGCTT |
| | | TATAAGATTAATGCAAAACTCTGTGATCCTCATCCCTCCAAGAAAGGAGCA |
| | | AGCTCAGCTTACCTTGAGAACTCGAAAGGTGCCCCCAACAACTCCTGCTCT |
| | | GAGATAAAAATGAACAAGAAAGGCGCTAGAATTGATTTTAAAGATGTGACT |
| | | TACTTAACAGAAGAGAAGGTATATGAAATTCTTGAATTATGTAGAGAAAGA |
| | | GAGGATTATTCCCCTTTAATCCGTGTTATTGGAAGAGTTTTTTCTAGTGCT |
| | | GAGGCATTGGTACAGAGCTTCCGGAAAGTTAAACAACACACCAAGGAAGAA |
| | | CTGAAATCTCTTCAAGCAAAAGATGAAGACAAAGATGAAGATGAAAAGGAA |
| | | AAAGCTGCATGTTCTGCTGCTGCTATGGAAGAAGACTCAGAAGCATCTTCC |
| | | TCAAGGATAGGTGATAGCTCACAGGGAGACAACAATTTGCAAAAATTAGGC |
| | | CCTGATGATGTGTCTGTGGATATTGATGCCATTAGAAGGGTCTACACCAGA |
| | | TTGCTCTCTAATGAAAAAATTGAAACTGCCTTTCTCAATGCACTTGTATAT |
| | | TTGTCACCTAACGTGGAATGTGACTTGACGTATCACAATGTATACTCTCGA |
| | | GATCCTAATTATCTGAATTTGTTCATTATCGTAATGGAGAATAGAAATCTC |
| | | CACAGTCCTGAATATCTGGAAATGGCTTTGCCATTATTTTGCAAAGCGATG |
| | | AGCAAGCTACCCCTTGCAGCCCAAGGAAAACTGATCAGACTGTGGTCTAAA |
| | | TACAATGCAGACCAGATTCGGAGAATGATGGAGACATTTCAGCAACTTATT |
| | | ACTTATAAAGTCATAAGCAATGAATTTAACAGTCGAAATCTAGTGAATGAT |
| | | GATGATGCCATTGTTGCTGCTTCGAAGTGCTTGAAAATGGTTTACTATGCA |
| | | AATGTAGTGGGAGGGGAAGTGGACACAAATCACAATGAAGAAGATGATGAA |
| | | GAGCCCATCCCTGAGTCCAGCGAGCTGACACTTCAGGAACTTTTGGGAGAA |
| | | GAAAGAAGAAACAAGAAAGGTCCTCGAGTGGACCCCCTGGAAACTGAACTT |
| | | GGTGTTAAAACCCTGGATTGTCGAAAACCACTTATCCCTTTTGAAGAGTTT |
| | | ATTAATGAACCACTGAATGAGGTTCTAGAAATGGATAAAGATTATACTTTT |
| | | TTCAAAGTAGAAACAGAGAACAAATTCTCTTTTATGACATGTCCCTTTATA |
| | | TTGAATGCTGTCACAAAGAATTTGGGATTATATTATGACAATAGAATTCGC |
| | | ATGTACAGTGAACGAAGAATCACTGTTCTCTACAGCTTAGTTCAAGGACAG |
| | | CAGTTGAATCCATATTTGAGACTCAAAGTTAGACGTGACCATATCATAGAT |
| | | GATGCACTTGTCCGGCTAGAGATGATCGCTATGGAAAATCCTGCAGACTTG |
| | | AAGAAGCAGTTGTATGTGGAATTTGAAGGAGAACAAGGAGTTGATGAGGGA |
| | | GGTGTTTCCAAAGAATTTTTTCAGCTGGTTGTGGAGGAAATCTTCAATCCA |
| | | GATATTGGTATGTTCACATACGATGAATCTACAAAATTGTTTTGGTTTAAT |
| | | CCATCTTCTTTTGAAACTGAGGGTCAGTTTACTCTGATTGGCATAGTACTG |
| | | GGTCTGGCTATTTACAATAACTGTATACTGGATGTACATTTTCCCATGGTT |
| | | GTCTACAGGAAGCTAATGGGGAAAAAAGGAACTTTTCGTGACTTGGGAGAC |
| | | TCTCACCCAGTTCTATATCAGAGTTTAAAAGATTTATTGGAGTATGAAGGG |
| | | AATGTGGAAGATGACATGATGATCACTTTCCAGATATCACAGACAGATCTT |
| | | TTTGGTAACCCAATGATGTATGATCTAAAGGAAAATGGTGATAAAATTCCA |
| | | ATTACAAATGAAAACAGGAAGGAATTTGTCAATCTTTATTCTGACTACATT |
| | | CTCAATAAATCAGTAGAAAAACAGTTCAAGGCTTTTCGGAGAGGTTTTCAT |
| | | ATGGTGACCAATGAATCTCCCTTAAAGTACTTATTCAGACCAGAAGAAATT |
| | | GAATTGCTTATATGTGGAAGCCGGAATCTAGATTTCCAAGCACTAGAAGAA |
| | | ACTACAGAATATGACGGTGGCTATACCAGGGACTCTGTTCTGATTAGGGAG |
| | | TTCTGGGAAATCGTTCATTCATTTACAGATGAACAGAAAAGACTCTTCTTG |
| | | CAGTTTACAACGGGCACAGACAGAGCACCTGTGGGAGGACTAGGAAAATTA |
| | | AAGATGATTATAGCCAAAAATGGCCCAGACACAGAAAGGTTACCTACATCT |
| | | CATACTTGCTTTAATGTGCTTTTACTTCCGGAATACTCAAGCAAAGAAAAA |
| | | CTTAAAGAGAGATTGTTGAAGGCCATCACGTATGCCAAAGGATTTGGCATG |
| | | CTCTAAGTTTAAACGATTCGAACTGTGCCTTCTAGTTGCCAGCCATCTGTT |
| | | GTTTGCCCCTCCCCCGTGCCTTCCTTGACCCTGGAAGGTGCCACTCCCACT |
| | | GTCCTTTCCTAATAAAATGAGGAAATTGCATCGCATTGTCTGAGTAGGTGT |
| | | CATTCTATTCTGGGGGGTGGGGTGGGGCAGGACAGCAAGGGGGAGGATTGG |
| | | GAAGACAATAGCAGGCATGCTGGGGATGCGGTGGGCTCTATGGGTGAAGTG |
| | | GGTAACCTTTATTTCCCTTCTTTTTCTCTTTAGCTCTGCTTATTCCAGGGG |
| | | TGTGTTTAGGAGAGATGCACACAAGAGTGAGGTTGCTCATAGGTTTAAAGA |
| | | TTTGGGAGAAGAAAATTTCAAAGCCTTGGTGTTGATTGCCTTTGCTCAGTA |
| | | TCTTCAGCAGTGTCCATTTGAAGATCATGTAAAATTAGTGAATGAAGTAAC |
| | | TGAATTTGCAAAAACATGTGTTGCTGATGAGTCAGCTGAAAATTGTGACAA |
| | | ATCACTTCATACCCTTTTTGGAGACAAATTATGCACAGTTGCAACTCTTAG |
| | | GGAAACCTATGGTGAATAGGCTGACTGCTGTGCAAAACAAGAACCTGAGAG |
| | | AAATGAATGCTTCTTGCAACACAAAGATGACAACCCAAACCTCCCCAGATT |
| | | GGTGAGACCAGAGGTTGATGTGTAGTGCACTGCTTTTCATGACAATGAAGA |
| | | GACCTTTTTGAAAAAATACTTATATGAAATTGCCAGAAGACATCCTTACTT |
| | | TTATGCCCCTGAACTCCTTTTCTTTGCTAAAAGGTATAAAGCTGCTTTTAC |
| | | AGAATGTTGCCAAGCTGCTGATAAAGCTGCCGGCGCGCCATCGATGAGGAA |
| | | CCCCTAGTGATGGAGTTGGCCACTCCCTCTCTGCGCGCTCGCTCGCTCACT |
| | | GAGGCCGGGCGACCAAAGGTCGCCCGACGCCCGGGCTTTGCCCGGGCGGCC |
| | | TCAGTGAGCGAGCGAGCGCGCAGAGAGGGAGTGGCCAA |
| 2 | 5′-ITR 145 bp | TTGGCCACTCCCTCTCTGCGCGCTCGCTCGCTCACTGAGGCCGGGCGACCA |
| | | AAGGTCGCCCGACGCCCGGGCTTTGCCCGGGCGGCCTCAGTGAGCGAGCGA |
| | | GCGCGCAGAGAGGGAGTGGCCAACTCCATCACTAGGGGTTCCT |
| 3 | hSyn Promoter | AGGGCCCTGCGTATGAGTGCAAGTGGGTTTTAGGACCAGGATGAGGCGGGG |
| | | TGGGGGTGCCTACCTGACGACCGACCCCGACCCACTGGACAAGCACCCAAC |
| | | CCCCATTCCCCAAATTGCGCATCCCCTATCAGAGAGGGGGAGGGGAAACAG |
| | | GATGCGGCGAGGCGCGTGCGCACTGCCAGCTTCAGCACCGCGGACAGTGCC |
| | | TTCGCCCCCGCCTGGCGGCGCGCGCCACCGCCGCCTCAGCACTGAAGGCGC |
| | | GCTGACGTCACTCGCCGGTCCCCCGCAAACTCCCCTTCCCGGCCACCTTGG |
| | | TCGCGTCCGCGCCGCCGCCGGCCCAGCCGGACCGCACCACGCGAGGCGCGA |
| | | GATAGGGGGGCACGGGCGCGACCATCTGCGCTGCGGCGCCGGCGACTCAGC |
| | | GCTGCCTCAGTCTGCGGTGGGCAGCGGAGGAGTCGTGTCGTGCCTGAGAGC |
| | | GCAG |
| 4 | hBG intron v2 | AGGGTGAGTCTATGGGACCCTTGATGTTTTCTTTCCCCTTCTTTTCTATGG |
| | | TTAAGTTCATGTCATAGGAAGGGGATAAGTAACAGGGTACACATATTGACC |
| | | AAATCAGGGTAATTTTGCATTTGTAATTTTAAAAAATGCTTTCTTCTTTTA |
| | | ATATACTTTTTTGTTTATCTTATTTCTAATACTTTCCCTAATCTCTTTCTT |
| | | TCAAGGCAATAATGATACAATGTATCATGCCTCTTTGCACCATTCTAAAGA |
| | | ATAACAGTGATAATTTCTGGGTTAAGGCAATAGCAATATTTCTGCATATAA |
| | | ATATTTCTGCATATAAATTGTAACTGATGTAAGAGGTTTCATATTGCTAAT |
| | | AGCAGCTACAATCCAGCTACCATTCTGCTTTTATTTTATGGTTGGGATAAG |
| | | GCTGGATTATTCTGAGTCCAAGCTAGGCCCTTTTGCTAATCATGTTCATAC |
| | | CTCTTATCTTCCTCCCACAGCTC |
| 5 | hUBE3Av2 | ATGAAGCGAGCAGCTGCAAAGCATCTAATAGAACGCTACTACCACCAGTTA |
| | | ACTGAGGGCTGTGGAAATGAAGCCTGCACGAATGAGTTTTGTGCTTCCTGT |
| | | CCAACTTTTCTTCGTATGGATAATAATGCAGCAGCTATTAAAGCCCTCGAG |
| | | CTTTATAAGATTAATGCAAAACTCTGTGATCCTCATCCCTCCAAGAAAGGA |
| | | GCAAGCTCAGCTTACCTTGAGAACTCGAAAGGTGCCCCCAACAACTCCTGC |
| | | TCTGAGATAAAAATGAACAAGAAAGGCGCTAGAATTGATTTTAAAGATGTG |
| | | ACTTACTTAACAGAAGAGAAGGTATATGAAATTCTTGAATTATGTAGAGAA |
| | | AGAGAGGATTATTCCCCTTTAATCCGTGTTATTGGAAGAGTTTTTTCTAGT |
| | | GCTGAGGCATTGGTACAGAGCTTCCGGAAAGTTAAACAACACACCAAGGAA |
| | | GAACTGAAATCTCTTCAAGCAAAAGATGAAGACAAAGATGAAGATGAAAAG |
| | | GAAAAAGCTGCATGTTCTGCTGCTGCTATGGAAGAAGACTCAGAAGCATCT |
| | | TCCTCAAGGATAGGTGATAGCTCACAGGGAGACAACAATTTGCAAAAATTA |
| | | GGCCCTGATGATGTGTCTGTGGATATTGATGCCATTAGAAGGGTCTACACC |
| | | AGATTGCTCTCTAATGAAAAAATTGAAACTGCCTTTCTCAATGCACTTGTA |
| | | TATTTGTCACCTAACGTGGAATGTGACTTGACGTATCACAATGTATACTCT |
| | | CGAGATCCTAATTATCTGAATTTGTTCATTATCGTAATGGAGAATAGAAAT |
| | | CTCCACAGTCCTGAATATCTGGAAATGGCTTTGCCATTATTTTGCAAAGCG |
| | | ATGAGCAAGCTACCCCTTGCAGCCCAAGGAAAACTGATCAGACTGTGGTCT |
| | | AAATACAATGCAGACCAGATTCGGAGAATGATGGAGACATTTCAGCAACTT |
| | | ATTACTTATAAAGTCATAAGCAATGAATTTAACAGTCGAAATCTAGTGAAT |
| | | GATGATGATGCCATTGTTGCTGCTTCGAAGTGCTTGAAAATGGTTTACTAT |
| | | GCAAATGTAGTGGGAGGGGAAGTGGACACAAATCACAATGAAGAAGATGAT |
| | | GAAGAGCCCATCCCTGAGTCCAGCGAGCTGACACTTCAGGAACTTTTGGGA |
| | | GAAGAAAGAAGAAACAAGAAAGGTCCTCGAGTGGACCCCCTGGAAACTGAA |
| | | CTTGGTGTTAAAACCCTGGATTGTCGAAAACCACTTATCCCTTTTGAAGAG |
| | | TTTATTAATGAACCACTGAATGAGGTTCTAGAAATGGATAAAGATTATACT |
| | | TTTTTCAAAGTAGAAACAGAGAACAAATTCTCTTTTATGACATGTCCCTTT |
| | | ATATTGAATGCTGTCACAAAGAATTTGGGATTATATTATGACAATAGAATT |
| | | CGCATGTACAGTGAACGAAGAATCACTGTTCTCTACAGCTTAGTTCAAGGA |
| | | CAGCAGTTGAATCCATATTTGAGACTCAAAGTTAGACGTGACCATATCATA |
| | | GATGATGCACTTGTCCGGCTAGAGATGATCGCTATGGAAAATCCTGCAGAC |
| | | TTGAAGAAGCAGTTGTATGTGGAATTTGAAGGAGAACAAGGAGTTGATGAG |
| | | GGAGGTGTTTCCAAAGAATTTTTTCAGCTGGTTGTGGAGGAAATCTTCAAT |
| | | CCAGATATTGGTATGTTCACATACGATGAATCTACAAAATTGTTTTGGTTT |
| | | AATCCATCTTCTTTTGAAACTGAGGGTCAGTTTACTCTGATTGGCATAGTA |
| | | CTGGGTCTGGCTATTTACAATAACTGTATACTGGATGTACATTTTCCCATG |
| | | GTTGTCTACAGGAAGCTAATGGGGAAAAAAGGAACTTTTCGTGACTTGGGA |
| | | GACTCTCACCCAGTTCTATATCAGAGTTTAAAAGATTTATTGGAGTATGAA |
| | | GGGAATGTGGAAGATGACATGATGATCACTTTCCAGATATCACAGACAGAT |
| | | CTTTTTGGTAACCCAATGATGTATGATCTAAAGGAAAATGGTGATAAAATT |
| | | CCAATTACAAATGAAAACAGGAAGGAATTTGTCAATCTTTATTCTGACTAC |
| | | ATTCTCAATAAATCAGTAGAAAAACAGTTCAAGGCTTTTCGGAGAGGTTTT |
| | | CATATGGTGACCAATGAATCTCCCTTAAAGTACTTATTCAGACCAGAAGAA |
| | | ATTGAATTGCTTATATGTGGAAGCCGGAATCTAGATTTCCAAGCACTAGAA |
| | | GAAACTACAGAATATGACGGTGGCTATACCAGGGACTCTGTTCTGATTAGG |
| | | GAGTTCTGGGAAATCGTTCATTCATTTACAGATGAACAGAAAAGACTCTTC |
| | | TTGCAGTTTACAACGGGCACAGACAGAGCACCTGTGGGAGGACTAGGAAAA |
| | | TTAAAGATGATTATAGCCAAAAATGGCCCAGACACAGAAAGGTTACCTACA |
| | | TCTCATACTTGCTTTAATGTGCTTTTACTTCCGGAATACTCAAGCAAAGAA |
| | | AAACTTAAAGAGAGATTGTTGAAGGCCATCACGTATGCCAAAGGATTTGGC |
| | | ATGCTCTAA |
| 6 | bGH polyA signal | CTGTGCCTTCTAGTTGCCAGCCATCTGTTGTTTGCCCCTCCCCCGTGCCTT |
| | | CCTTGACCCTGGAAGGTGCCACTCCCACTGTCCTTTCCTAATAAAATGAGG |
| | | AAATTGCATCGCATTGTCTGAGTAGGTGTCATTCTATTCTGGGGGGTGGGG |
| | | TGGGGCAGGACAGCAAGGGGGAGGATTGGGAAGACAATAGCAGGCATGCTG |
| | | GGGATGCGGTGGGCTCTATGG |
| 7 | hAlb Stuffer | GTGAAGTGGGTAACCTTTATTTCCCTTCTTTTTCTCTTTAGCTCTGCTTAT |
| | | TCCAGGGGTGTGTTTAGGAGAGATGCACACAAGAGTGAGGTTGCTCATAGG |
| | | TTTAAAGATTTGGGAGAAGAAAATTTCAAAGCCTTGGTGTTGATTGCCTTT |
| | | GCTCAGTATCTTCAGCAGTGTCCATTTGAAGATCATGTAAAATTAGTGAAT |
| | | GAAGTAACTGAATTTGCAAAAACATGTGTTGCTGATGAGTCAGCTGAAAAT |
| | | TGTGACAAATCACTTCATACCCTTTTTGGAGACAAATTATGCACAGTTGCA |
| | | ACTCTTAGGGAAACCTATGGTGAATAGGCTGACTGCTGTGCAAAACAAGAA |
| | | CCTGAGAGAAATGAATGCTTCTTGCAACACAAAGATGACAACCCAAACCTC |
| | | CCCAGATTGGTGAGACCAGAGGTTGATGTGTAGTGCACTGCTTTTCATGAC |
| | | AATGAAGAGACCTTTTTGAAAAAATACTTATATGAAATTGCCAGAAGACAT |
| | | CCTTACTTTTATGCCCCTGAACTCCTTTTCTTTGCTAAAAGGTATAAAGCT |
| | | GCTTTTACAGAATGTTGCCAAGCTGCTGATAAAGCTGCC |
| 8 | 3′-ITR 145 bp | AGGAACCCCTAGTGATGGAGTTGGCCACTCCCTCTCTGCGCGCTCGCTCGC |
| | | TCACTGAGGCCGGGCGACCAAAGGTCGCCCGACGCCCGGGCTTTGCCCGGG |
| | | CGGCCTCAGTGAGCGAGCGAGCGCGCAGAGAGGGAGTGGCCAA |
| 9 | 5′-ITR 141 bp | CCTGCAGGCAGCTGCGCGCTCGCTCGCTCACTGAGGCCGCCCGGGCAAAGC |
| | | CCGGGCGTCGGGCGACCTTTGGTCGCCCGGCCTCAGTGAGCGAGCGAGCGC |
| | | GCAGAGAGGGAGTGGCCAACTCCATCACTAGGGGTTCCT |
| 10 | 3′-ITR 141 bp | AGGAACCCCTAGTGATGGAGTTGGCCACTCCCTCTCTGCGCGCTCGCTCGC |
| | | TCACTGAGGCCGGGCGACCAAAGGTCGCCCGACGCCCGGGCTTTGCCCGGG |
| | | CGGCCTCAGTGAGCGAGCGAGCGCGCAGCTGCCTGCAGG |
| 11 | pTR145K-hSyn- | CCTGCAGGCAGCTGCGCGCTCGCTCGCTCACTGAGGCCGCCCGGGCAAAGC |
| | hBGINv2- | CCGGGCGTCGGGCGACCTTTGGTCGCCCGGCCTCAGTGAGCGAGCGAGCGC |
| | hUBE3Av2-bGHpA- | GCAGAGAGGGAGTGGCCAACTCCATCACTAGGGGTTCCTTGCGTCGACAGG |
| | hAlb with | GCCCTGCGTATGAGTGCAAGTGGGTTTTAGGACCAGGATGAGGCGGGGTGG |
| | truncated | GGGTGCCTACCTGACGACCGACCCCGACCCACTGGACAAGCACCCAACCCC |
| | ITRs | CATTCCCCAAATTGCGCATCCCCTATCAGAGAGGGGGAGGGGAAACAGGAT |
| | | GCGGCGAGGCGCGTGCGCACTGCCAGCTTCAGCACCGCGGACAGTGCCTTC |
| | | GCCCCCGCCTGGCGGCGCGCGCCACCGCCGCCTCAGCACTGAAGGCGCGCT |
| | | GACGTCACTCGCCGGTCCCCCGCAAACTCCCCTTCCCGGCCACCTTGGTCG |
| | | CGTCCGCGCCGCCGCCGGCCCAGCCGGACCGCACCACGCGAGGCGCGAGAT |
| | | AGGGGGGCACGGGCGCGACCATCTGCGCTGCGGCGCCGGCGACTCAGCGCT |
| | | GCCTCAGTCTGCGGTGGGCAGCGGAGGAGTCGTGTCGTGCCTGAGAGCGCA |
| | | GAAGCTTAGGGTGAGTCTATGGGACCCTTGATGTTTTCTTTCCCCTTCTTT |
| | | TCTATGGTTAAGTTCATGTCATAGGAAGGGGATAAGTAACAGGGTACACAT |
| | | ATTGACCAAATCAGGGTAATTTTGCATTTGTAATTTTAAAAAATGCTTTCT |
| | | TCTTTTAATATACTTTTTTGTTTATCTTATTTCTAATACTTTCCCTAATCT |
| | | CTTTCTTTCAAGGCAATAATGATACAATGTATCATGCCTCTTTGCACCATT |
| | | CTAAAGAATAACAGTGATAATTTCTGGGTTAAGGCAATAGCAATATTTCTG |
| | | CATATAAATATTTCTGCATATAAATTGTAACTGATGTAAGAGGTTTCATAT |
| | | TGCTAATAGCAGCTACAATCCAGCTACCATTCTGCTTTTATTTTATGGTTG |
| | | GGATAAGGCTGGATTATTCTGAGTCCAAGCTAGGCCCTTTTGCTAATCATG |
| | | TTCATACCTCTTATCTTCCTCCCACAGCTCCACCGGTCGCCACCATGAAGC |
| | | GAGCAGCTGCAAAGCATCTAATAGAACGCTACTACCACCAGTTAACTGAGG |
| | | GCTGTGGAAATGAAGCCTGCACGAATGAGTTTTGTGCTTCCTGTCCAACTT |
| | | TTCTTCGTATGGATAATAATGCAGCAGCTATTAAAGCCCTCGAGCTTTATA |
| | | AGATTAATGCAAAACTCTGTGATCCTCATCCCTCCAAGAAAGGAGCAAGCT |
| | | CAGCTTACCTTGAGAACTCGAAAGGTGCCCCCAACAACTCCTGCTCTGAGA |
| | | TAAAAATGAACAAGAAAGGCGCTAGAATTGATTTTAAAGATGTGACTTACT |
| | | TAACAGAAGAGAAGGTATATGAAATTCTTGAATTATGTAGAGAAAGAGAGG |
| | | ATTATTCCCCTTTAATCCGTGTTATTGGAAGAGTTTTTTCTAGTGCTGAGG |
| | | CATTGGTACAGAGCTTCCGGAAAGTTAAACAACACACCAAGGAAGAACTGA |
| | | AATCTCTTCAAGCAAAAGATGAAGACAAAGATGAAGATGAAAAGGAAAAAG |
| | | CTGCATGTTCTGCTGCTGCTATGGAAGAAGACTCAGAAGCATCTTCCTCAA |
| | | GGATAGGTGATAGCTCACAGGGAGACAACAATTTGCAAAAATTAGGCCCTG |
| | | ATGATGTGTCTGTGGATATTGATGCCATTAGAAGGGTCTACACCAGATTGC |
| | | TCTCTAATGAAAAAATTGAAACTGCCTTTCTCAATGCACTTGTATATTTGT |
| | | CACCTAACGTGGAATGTGACTTGACGTATCACAATGTATACTCTCGAGATC |
| | | CTAATTATCTGAATTTGTTCATTATCGTAATGGAGAATAGAAATCTCCACA |
| | | GTCCTGAATATCTGGAAATGGCTTTGCCATTATTTTGCAAAGCGATGAGCA |
| | | AGCTACCCCTTGCAGCCCAAGGAAAACTGATCAGACTGTGGTCTAAATACA |
| | | ATGCAGACCAGATTCGGAGAATGATGGAGACATTTCAGCAACTTATTACTT |
| | | ATAAAGTCATAAGCAATGAATTTAACAGTCGAAATCTAGTGAATGATGATG |
| | | ATGCCATTGTTGCTGCTTCGAAGTGCTTGAAAATGGTTTACTATGCAAATG |
| | | TAGTGGGAGGGGAAGTGGACACAAATCACAATGAAGAAGATGATGAAGAGC |
| | | CCATCCCTGAGTCCAGCGAGCTGACACTTCAGGAACTTTTGGGAGAAGAAA |
| | | GAAGAAACAAGAAAGGTCCTCGAGTGGACCCCCTGGAAACTGAACTTGGTG |
| | | TTAAAACCCTGGATTGTCGAAAACCACTTATCCCTTTTGAAGAGTTTATTA |
| | | ATGAACCACTGAATGAGGTTCTAGAAATGGATAAAGATTATACTTTTTTCA |
| | | AAGTAGAAACAGAGAACAAATTCTCTTTTATGACATGTCCCTTTATATTGA |
| | | ATGCTGTCACAAAGAATTTGGGATTATATTATGACAATAGAATTCGCATGT |
| | | ACAGTGAACGAAGAATCACTGTTCTCTACAGCTTAGTTCAAGGACAGCAGT |
| | | TGAATCCATATTTGAGACTCAAAGTTAGACGTGACCATATCATAGATGATG |
| | | CACTTGTCCGGCTAGAGATGATCGCTATGGAAAATCCTGCAGACTTGAAGA |
| | | AGCAGTTGTATGTGGAATTTGAAGGAGAACAAGGAGTTGATGAGGGAGGTG |
| | | TTTCCAAAGAATTTTTTCAGCTGGTTGTGGAGGAAATCTTCAATCCAGATA |
| | | TTGGTATGTTCACATACGATGAATCTACAAAATTGTTTTGGTTTAATCCAT |
| | | CTTCTTTTGAAACTGAGGGTCAGTTTACTCTGATTGGCATAGTACTGGGTC |
| | | TGGCTATTTACAATAACTGTATACTGGATGTACATTTTCCCATGGTTGTCT |
| | | ACAGGAAGCTAATGGGGAAAAAAGGAACTTTTCGTGACTTGGGAGACTCTC |
| | | ACCCAGTTCTATATCAGAGTTTAAAAGATTTATTGGAGTATGAAGGGAATG |
| | | TGGAAGATGACATGATGATCACTTTCCAGATATCACAGACAGATCTTTTTG |
| | | GTAACCCAATGATGTATGATCTAAAGGAAAATGGTGATAAAATTCCAATTA |
| | | CAAATGAAAACAGGAAGGAATTTGTCAATCTTTATTCTGACTACATTCTCA |
| | | ATAAATCAGTAGAAAAACAGTTCAAGGCTTTTCGGAGAGGTTTTCATATGG |
| | | TGACCAATGAATCTCCCTTAAAGTACTTATTCAGACCAGAAGAAATTGAAT |
| | | TGCTTATATGTGGAAGCCGGAATCTAGATTTCCAAGCACTAGAAGAAACTA |
| | | CAGAATATGACGGTGGCTATACCAGGGACTCTGTTCTGATTAGGGAGTTCT |
| | | GGGAAATCGTTCATTCATTTACAGATGAACAGAAAAGACTCTTCTTGCAGT |
| | | TTACAACGGGCACAGACAGAGCACCTGTGGGAGGACTAGGAAAATTAAAGA |
| | | TGATTATAGCCAAAAATGGCCCAGACACAGAAAGGTTACCTACATCTCATA |
| | | CTTGCTTTAATGTGCTTTTACTTCCGGAATACTCAAGCAAAGAAAAACTTA |
| | | AAGAGAGATTGTTGAAGGCCATCACGTATGCCAAAGGATTTGGCATGCTCT |
| | | AAGTTTAAACGATTCGAACTGTGCCTTCTAGTTGCCAGCCATCTGTTGTTT |
| | | GCCCCTCCCCCGTGCCTTCCTTGACCCTGGAAGGTGCCACTCCCACTGTCC |
| | | TTTCCTAATAAAATGAGGAAATTGCATCGCATTGTCTGAGTAGGTGTCATT |
| | | CTATTCTGGGGGGTGGGGTGGGGCAGGACAGCAAGGGGGAGGATTGGGAAG |
| | | ACAATAGCAGGCATGCTGGGGATGCGGTGGGCTCTATGGGTGAAGTGGGTA |
| | | ACCTTTATTTCCCTTCTTTTTCTCTTTAGCTCTGCTTATTCCAGGGGTGTG |
| | | TTTAGGAGAGATGCACACAAGAGTGAGGTTGCTCATAGGTTTAAAGATTTG |
| | | GGAGAAGAAAATTTCAAAGCCTTGGTGTTGATTGCCTTTGCTCAGTATCTT |
| | | CAGCAGTGTCCATTTGAAGATCATGTAAAATTAGTGAATGAAGTAACTGAA |
| | | TTTGCAAAAACATGTGTTGCTGATGAGTCAGCTGAAAATTGTGACAAATCA |
| | | CTTCATACCCTTTTTGGAGACAAATTATGCACAGTTGCAACTCTTAGGGAA |
| | | ACCTATGGTGAATAGGCTGACTGCTGTGCAAAACAAGAACCTGAGAGAAAT |
| | | GAATGCTTCTTGCAACACAAAGATGACAACCCAAACCTCCCCAGATTGGTG |
| | | AGACCAGAGGTTGATGTGTAGTGCACTGCTTTTCATGACAATGAAGAGACC |
| | | TTTTTGAAAAAATACTTATATGAAATTGCCAGAAGACATCCTTACTTTTAT |
| | | GCCCCTGAACTCCTTTTCTTTGCTAAAAGGTATAAAGCTGCTTTTACAGAA |
| | | TGTTGCCAAGCTGCTGATAAAGCTGCCGGCGCGCCATCGATGAGGAACCCC |
| | | TAGTGATGGAGTTGGCCACTCCCTCTCTGCGCGCTCGCTCGCTCACTGAGG |
| | | CCGGGCGACCAAAGGTCGCCCGACGCCCGGGCTTTGCCCGGGCGGCCTCAG |
| | | TGAGCGAGCGAGCGCGCAGCTGCCTGCAGG |
| 12 | Wild type UBE3A | atgaagcgagcagctgcaaagcatctaatagaacgctactaccaccagtta |
| | human gene | actgagggctgtggaaatgaagcctgcacgaatgagttttgtgcttcctgt |
| | | ccaacttttcttcgtatggataataatgcagcagctattaaagccctcgag |
| | | ctttataagattaatgcaaaactctgtgatcctcatccctccaagaaagga |
| | | gcaagctcagcttaccttgagaactcgaaaggtgcccccaacaactcctgc |
| | | tctgagataaaaatgaacaagaaaggcgctagaattgattttaaagatgtg |
| | | acttacttaacagaagagaaggtatatgaaattcttgaattatgtagagaa |
| | | agagaggattattcccctttaatccgtgttattggaagagttttttctagt |
| | | gctgaggcattggtacagagcttccggaaagttaaacaacacaccaaggaa |
| | | gaactgaaatctcttcaagcaaaagatgaagacaaagatgaagatgaaaag |
| | | gaaaaagctgcatgttctgctgctgctatggaagaagactcagaagcatct |
| | | tcctcaaggataggtgatagctcacagggagacaacaatttgcaaaaatta |
| | | ggccctgatgatgtgtctgtggatattgatgccattagaagggtctacacc |
| | | agattgctctctaatgaaaaaattgaaactgcctttctcaatgcacttgta |
| | | tatttgtcacctaacgtggaatgtgacttgacgtatcacaatgtatactct |
| | | cgagatcctaattatctgaatttgttcattatcgtaatggagaatagaaat |
| | | ctccacagtcctgaatatctggaaatggctttgccattattttgcaaagcg |
| | | atgagcaagctaccccttgcagcccaaggaaaactgatcagactgtggtct |
| | | aaatacaatgcagaccagattcggagaatgatggagacatttcagcaactt |
| | | attacttataaagtcataagcaatgaatttaacagtcgaaatctagtgaat |
| | | gatgatgatgccattgttgctgcttcgaagtgcttgaaaatggtttactat |
| | | gcaaatgtagtgggaggggaagtggacacaaatcacaatgaagaagatgat |
| | | gaagagcccatccctgagtccagcgagctgacacttcaggaacttttggga |
| | | gaagaaagaagaaacaagaaaggtcctcgagtggaccccctggaaactgaa |
| | | cttggtgttaaaaccctggattgtcgaaaaccacttatcccttttgaagag |
| | | tttattaatgaaccactgaatgaggttctagaaatggataaagattatact |
| | | tttttcaaagtagaaacagagaacaaattctcttttatgacatgtcccttt |
| | | atattgaatgctgtcacaaagaatttgggattatattatgacaatagaatt |
| | | cgcatgtacagtgaacgaagaatcactgttctctacagcttagttcaagga |
| | | cagcagttgaatccatatttgagactcaaagttagacgtgaccatatcata |
| | | gatgatgcacttgtccggctagagatgatcgctatggaaaatcctgcagac |
| | | ttgaagaagcagttgtatgtggaatttgaaggagaacaaggagttgatgag |
| | | ggaggtgtttccaaagaattttttcagctggttgtggaggaaatcttcaat |
| | | ccagatattggtatgttcacatacgatgaatctacaaaattgttttggttt |
| | | aatccatcttcttttgaaactgagggtcagtttactctgattggcatagta |
| | | ctgggtctggctatttacaataactgtatactggatgtacattttcccatg |
| | | gttgtctacaggaagctaatggggaaaaaaggaacttttcgtgacttggga |
| | | gactctcacccagttctatatcagagtttaaaagatttattggagtatgaa |
| | | gggaatgtggaagatgacatgatgatcactttccagatatcacagacagat |
| | | ctttttggtaacccaatgatgtatgatctaaaggaaaatggtgataaaatt |
| | | ccaattacaaatgaaaacaggaaggaatttgtcaatctttattctgactac |
| | | attctcaataaatcagtagaaaaacagttcaaggcttttcggagaggtttt |
| | | catatggtgaccaatgaatctcccttaaagtacttattcagaccagaagaa |
| | | attgaattgcttatatgtggaagccggaatctagatttccaagcactagaa |
| | | gaaactacagaatatgacggtggctataccagggactctgttctgattagg |
| | | gagttctgggaaatcgttcattcatttacagatgaacagaaaagactcttc |
| | | ttgcagtttacaacgggcacagacagagcacctgtgggaggactaggaaaa |
| | | ttaaagatgattatagccaaaaatggcccagacacagaaaggttacctaca |
| | | tctcatacttgctttaatgtgcttttacttccggaatactcaagcaaagaa |
| | | aaacttaaagagagattgttgaaggccatcacgtatgccaaaggatttggc |
| | | atgctgtaa |
| 13 | unmodified hBGIN | gtgagtctatgggacccttgatgttttctttccccttcttttctatggtta |
| | | agttcatgtcataggaaggggagaagtaacagggtacacatattgaccaaa |
| | | tcagggtaattttgcatttgtaattttaaaaaatgctttcttcttttaata |
| | | tacttttttgtttatcttatttctaatactttccctaatctctttctttca |
| | | gggcaataatgatacaatgtatcatgcctctttgcaccattctaaagaata |
| | | acagtgataatttctgggttaaggcaatagcaatatttctgcatataaata |
| | | tttctgcatataaattgtaactgatgtaagaggtttcatattgctaatagc |
| | | agctacaatccagctaccattctgcttttattttatggttgggataaggct |
| | | ggattattctgagtccaagctaggcccttttgctaatcatgttcatacctc |
| | | ttatcttcctcccacag |
| 14 | Kozak sequence | gcgcgccatcgatgtgaagtgggtaacctttatttcccttctttttctctt |
| | | tagctcggcttattccaggggtgtgtttcgtcgagatgcacacaagagtga |
| | | ggttgctcatcggtttaaagatttgggagaagaaaatttcaaagccttggt |
| | | gttgattgcctttgctcagtatcttcagcagtgtccatttgaagatcatgt |
| | | aaaattagtgaatgaagtaactgaatttgcaaaaacatgtgttgctgatga |
| | | gtcagctgaaaattgtgacaaatcacttcataccctttttggagacaaatt |
| | | atgcacagttgcaactcttcgtgaaacctatggtgaaatggctgactgctg |
| | | tgcaaaacaagaacctgagagaaatgaatgcttcttgcaacacaaagatga |
| | | caacccaaacctcccccgattggtgagaccagaggttgatgtgatgtgcac |
| | | tgcttttcatgacaatgaagagacctttttgaaaaaatacttatacgaaat |
| | | tgccagaagacatccttacttttatgccccggaactccttttctttgctaa |
| | | aaggtataaagctgcttttacagaatgttgccaagctgctgataaagctgc |
| | | ctgcctgttgccaaagctcgatgaacttcgggatgaagggaaggcttcgtc |
| | | tgccaaacagagactcaagtgtgccagtctccaaaaatttggagaaagagc |
| | | tttcaaagcatgggcagtagctcgcctgagccagagatttcccaaagctga |
| | | gtttgcagaagtttccaagttagtgacagatcttaccaaagtccacacgga |
| | | atgctgccatggagatctgcttgaatgtgctgatgacagggcggaccttgc |
| | | caagtatatctgtgaaaatcaagattcgatctccagtaaactgaaggaatg |
| | | ctgtgaaaaacctctgttggaaaaatcccactgcattgccgaagtggaaaa |
| | | tgatgagatgcctgctgacttgccttcattagctgctgattttgttgaaag |
| | | taaggatgtttgcaaaaactatgctgaggcaaaggatgtcttcctgggcat |
| | | gtttttgtatgaatatgcaagaaggcatcctgattactctgtcgtgctgct |
| | | gctgagacttgccaagacctatgaaaccactctagagaagtgctgtgccgc |
| | | tgcagatcctcatgaatgctatgccaaagtgttcgatgaatttaaacctct |
| | | tgtggaagagcctcagaatttaatcaaacaaaattgtgagctttttgagca |
| | | gcttggagagtacaaattccagaatgcgctattagttcgttacaccaagaa |
| | | agtaccccaagtgtcaactccaactcttgtagaggtctcaagaaacctagg |
| | | aaaagtgggcagcaaatgttgtaaacatcctgaagcaaaaagaatgccctg |
| | | tgcagaagactatctatccgtggtcctgaaccagttatgtgtgttgcatga |
| | | gaaaacgccagtaagtgacagagtcaccaaatgctgcacagaatccttggt |
| | | gaacaggcgaccatgtcgtgaaacctatggtgaaatggctgactgctgtgc |
| | | aaaacaagaacctgagagaaatgaatgcttcttgcaacacaaagatgacaa |
| | | cccaaacctcccccgattggtgagaccagaggttgatgtgatgtgcactgc |
| | | ttttcatgacaatgaagagacctttttgaaaaaatacttatacgaaattgc |
| | | cagaagacatccttacttttatgccccggaactccttttctttgctaaaag |
| | | gtataaagctgcttttacagaatgttgccaagctgctgataaagctgcctg |
| | | cctgttgccaaagctcgatgaacttcgggatgaagggaaggcttcgtctgc |
| | | caaacagagactcaagtgtgccagtctccaaaaatttggagaaagagcttt |
| | | caaagcatgggcagtagctcgcctgagccagacttttcagctctggaagtc |
| | | gatgaaacatacgttcccaaagagtttaatgctgaaacattcaccttccat |
| | | gcagatatatgcacactttctgagaaggagagacaaatcaagaaacaaact |
| | | gcacttgttgagctcgtgaaacacaagcccaaggcaacaaaagagcaactg |
| | | aaagctgttatggatgatttcgc |
| 15 | STRV47 | MAADGYLPDWLEDNLSEGIREWWALKPGAPQPKANQQHQDNARGLVLPGYK |
| | | YLGPGNGLDKGEPVNAADAAALEHDKAYDQQLKAGDNPYLKYNHADAEFQE |
| | | RLKEDTSFGGNLGRAVFQAKKRLLEPLGLVEEAAKTAPGKKRPVEQSPQEP |
| | | DSSAGIGKSGAQPAKKRLNFGQTGDTESVPDPQPIGEPPAAPSGVGSLTMA |
| | | SGGGAPVADNNEGADGVGSSSGNWHCDSQWLGDRVITTSTRTWALPTYNNH |
| | | LYKQISNSTSGGSSNDNAYFGYSTPWGYFDFNRFHCHFSPRDWQRLINNNW |
| | | GFRPKRLNFKLFNIQVKEVTDNNGVKTIANNLTSTVQVFTDSDYQLPYVLG |
| | | SAHEGCLPPFPADVFMIPQYGYLTLNDGSQAVGRSSFYCLEYFPSQMLRTG |
| | | NNFQFSYEFENVPFHSSYAHSQSLDRLMNPLIDQYLYYLSKTIGVSLGGGQ |
| | | TLKFSVAGPSNMAVQGRNYIPGPSYRQQRVSTTVTQNNNSEFAWPGASSWA |
| | | LNGRNSLMNPGPAMASHKEGEDRFFPLSGSLIFGKQGTGRDNVDADKVMIT |
| | | NEEEIKTTNPVATESYGQVATNHQSAQAQAQTGWVQNQGILPGMVWQDRDV |
| | | YLQGPIWAKIPHTDGNFHPSPLMGGFGMKHPPPQILIKNTPVPADPPTAFN |
| | | KDKLNSFITQYSTGQVSVEIEWELQKENSKRWNPEIQYTSNYYKSNNVEFA |
| | | VNTEGVYSEPRPIGTRYLTRNL |
| 16 | STRV5 | MAADGYLPDWLEDNLSEGIREWWALKPGAPQPKANQQHQDNARGLVLPGYK |
| | | YLGPGNGLDKGEPVNAADAAALEHDKAYDQQLKAGDNPYLKYNHADAEFQE |
| | | RLKEDTSFGGNLGRAVFQAKKRLLEPLGLVEEAAKTAPGKKRPVEQSPQEP |
| | | DSSAGIGKSGAQPAKKRLNFGQTGDTESVPDPQPIGEPPAAPSGVGSLTMA |
| | | SGGGAPVADNNEGADGVGSSSGNWHCDSQWLGDRVITTSTRTWALPTYNNH |
| | | LYKQISNSTSGGSSNDNAYFGYSTPWGYFDFNRFHCHFSPRDWQRLINNNW |
| | | GFRPKRLNFKLFNIQVKEVTDNNGVKTIANNLTSTVQVFTDSDYQLPYVLG |
| | | SAHEGCLPPFPADVFMIPQYGYLTLNDGSQAVGRSSFYCLEYFPSQMLRTG |
| | | NNFQFSYEFENVPFHSSYAHSQSLDRLMNPLIDQYLYYLSKTRQDQPINAQ |
| | | TLKFSVAGPSNMAVQGRNYIPGPSYRQQRVSTTVTQNNNSEFAWPGASSWA |
| | | LNGRNSLMNPGPAMASHKEGEDRFFPLSGSLIFGKQGTGRDNVDADKVMIT |
| | | NEEEIKTTNPVATESYGQVATNHQSSKVESWTEWVQNQGILPGMVWQDRDV |
| | | YLQGPIWAKIPHTDGNFHPSPLMGGFGMKHPPPQILIKNTPVPADPPTAFN |
| | | KDKLNSFITQYSTGQVSVEIEWELQKENSKRWNPEIQYTSNYYKSNNVEFA |
| | | VNTEGVYSEPRPIGTRYLTRNL |
| 17 | STRV84 | MAADGYLPDWLEDNLSEGIREWWALKPGAPQPKANQQHQDNARGLVLPGYK |
| | | YLGPGNGLDKGEPVNAADAAALEHDKAYDQQLKAGDNPYLKYNHADAEFQE |
| | | RLKEDTSFGGNLGRAVFQAKKRLLEPLGLVEEAAKTAPGKKRPVEQSPQEP |
| | | DSSAGIGKSGAQPAKKRLNFGQTGDTESVPDPQPIGEPPAAPSGVGSLTMA |
| | | SGGGAPVADNNEGADGVGSSSGNWHCDSQWLGDRVITTSTRTWALPTYNNH |
| | | LYKQISNSTSGGSSNDNAYFGYSTPWGYFDFNRFHCHFSPRDWQRLINNNW |
| | | GFRPKRLNFKLFNIQVKEVTDNNGVKTIANNLTSTVQVFTDSDYQLPYVLG |
| | | SAHEGCLPPFPADVFMIPQYGYLTLNDGSQAVGRSSFYCLEYFPSQMLRTG |
| | | NNFQFSYEFENVPFHSSYAHSQSLDRLMNPLIDQYLYYLSKTINGSGQNQQ |
| | | TLKFSVAGPSNMAVQGRNYIPGPSYRQQRVSTTVTQNNNSEFAWPGASSWA |
| | | LNGRNSLMNPGPAMASHKEGEDRFFPLSGSLIFGKQGTGRDNVDADKVMIT |
| | | NEEEIKTTNPVATESYGQVATNHQNAVGALSTGWVQNQGILPGMVWQDRDV |
| | | YLQGPIWAKIPHTDGNFHPSPLMGGFGMKHPPPQILIKNTPVPADPPTAFN |
| | | KDKLNSFITQYSTGQVSVEIEWELQKENSKRWNPEIQYTSNYYKSNNVEFA |
| | | VNTEGVYSEPRPIGTRYLTRNL |
| 18 | CMV enhancer | tacataacttacggtaaatggcccgcctggctgaccgcccaacgacccccg |
| | | cccattgacgtcaataatgacgtatgttcccatagtaacgccaatagggac |
| | | tttccattgacgtcaatgggtggagtatttacggtaaactgcccacttggc |
| | | agtacatcaagtgtatcatatgccaagtacgccccctattgacgtcaatga |
| | | cggtaaatggcccgcctggcattatgcccagtacatgaccttatgggactt |
| | | tcctacttggcagtacatctacgtattagtcatcgctattaccatg |
| 19 | Human UBE3A | MKRAAAKHLIERYYHQLTEGCGNEACTNEFCASCPTFLRMDNNAAAIKALE |
| | protein sequence | LYKINAKLCDPHPSKKGASSAYLENSKGAPNNSCSEIKMNKKGARIDFKDV |
| | | TYLTEEKVYEILELCREREDYSPLIRVIGRVFSSAEALVQSFRKVKQHTKE |
| | | ELKSLQAKDEDKDEDEKEKAACSAAAMEEDSEASSSRIGDSSQGDNNLQKL |
| | | GPDDVSVDIDAIRRVYTRLLSNEKIETAFLNALVYLSPNVECDLTYHNVYS |
| | | RDPNYLNLFIIVMENRNLHSPEYLEMALPLFCKAMSKLPLAAQGKLIRLWS |
| | | KYNADQIRRMMETFQQLITYKVISNEFNSRNLVNDDDAIVAASKCLKMVYY |
| | | ANVVGGEVDTNHNEEDDEEPIPESSELTLQELLGEERRNKKGPRVDPLETE |
| | | LGVKTLDCRKPLIPFEEFINEPLNEVLEMDKDYTFFKVETENKFSFMTCPF |
| | | ILNAVTKNLGLYYDNRIRMYSERRITVLYSLVQGQQLNPYLRLKVRRDHII |
| | | DDALVRLEMIAMENPADLKKQLYVEFEGEQGVDEGGVSKEFFQLVVEEIEN |
| | | PDIGMFTYDESTKLFWFNPSSFETEGQFTLIGIVLGLAIYNNCILDVHFPM |
| | | VVYRKLMGKKGTFRDLGDSHPVLYQSLKDLLEYEGNVEDDMMITFQISQTD |
| | | LFGNPMMYDLKENGDKIPITNENRKEFVNLYSDYILNKSVEKQFKAFRRGF |
| | | HMVTNESPLKYLFRPEEIELLICGSRNLDFQALEETTEYDGGYTRDSVLIR |
| | | EFWEIVHSFTDEQKRLFLQFTTGTDRAPVGGLGKLKMIIAKNGPDTERLPT |
| | | SHTCFNVLLLPEYSSKEKLKERLLKAITYAKGFGML* |
| 20 | polyA signal | taagatacattgatgagtttggacaaaccacaactagaatgcagtgaaaaa |
| | | aatgctttatttgtgaaatttgtgatgctattgctttatttgtaaccatta |
| | | taagctgcaataaacaagtt |
| 21 | polyA signal | aataaaggaaatttattttcattgcaatagtgtgttggaattttttgtgtc |
| | | tctca |
| 22 | P-T116 sequence | tcgcgcgtttcggtgatgacggtgaaaacctctgacacatgcagctcccgg |
| | | agacggtcacagcttgtctgtaagcggatgccgggagcagacaagcccgtc |
| | | agggcgcgtcagcgggtgttggcgggtgtcggggctggcttaactatgcgg |
| | | catcagagcagattgtactgagagtgcacgatatcgggtccccaattgaca |
| | | ttattgaagcatttatcagggttattgtctcagaatttaaatgacgcctgc |
| | | aggcagctgcgcgctcgctcgctcactgaggccgcccgggcaaagcccggg |
| | | cgtcgggcgacctttggtcgcccggcctcagtgagcgagcgagcgcgcaga |
| | | gagggagtggccaactccatcactaggggttccttgcgTCGACagtgcaag |
| | | tgggttttaggaccaggatgaggcggggtgggggtgcctacctgacgaccg |
| | | accccgacccactggacaagcacccaacccccattccccaaattgcgcatc |
| | | ccctatcagagagggggaggggaaacaggatgcggcgaggcgcgtgcgcac |
| | | tgccagcttcagcaccgcggacagtgccttcgcccccgcctggcggcgcgc |
| | | gccaccgccgcctcagcactgaaggcgcgctgacgtcactcgccggtcccc |
| | | cgcaaactccccttcccggccaccttggtcgcgtccgcgccgccgccggcc |
| | | cagccggaccgcaccacgcgaggcgcgagataggggggcacgggcgcgacc |
| | | atctgcgctgcggcgccggcgactcagcgctgcctcagtctgcggtgggca |
| | | gcggaggagtcgtgtcgtgcctgagagcgcagatgcatgtaagtttagtct |
| | | ttttgtcttttatttcaggtcccggatccggtggtggtgcaaatcaaagaa |
| | | ctgctcctcagtggatgttgcctttacttctagcaccggtcgccaccatga |
| | | agcgagcagctgcaaagcatctaatagaacgctactaccaccagttaactg |
| | | agggctgtggaaatgaagcctgcacgaatgagttttgtgcttcctgtccaa |
| | | cttttcttcgtatggataataatgcagcagctattaaagccctcgagcttt |
| | | ataagattaatgcaaaactctgtgatcctcatccctccaagaaaggagcaa |
| | | gctcagcttaccttgagaactcgaaaggtgcccccaacaactcctgctctg |
| | | agataaaaatgaacaagaaaggcgctagaattgattttaaagatgtgactt |
| | | acttaacagaagagaaggtatatgaaattcttgaattatgtagagaaagag |
| | | aggattattcccctttaatccgtgttattggaagagttttttctagtgctg |
| | | aggcattggtacagagcttccggaaagttaaacaacacaccaaggaagaac |
| | | tgaaatctcttcaagcaaaagatgaagacaaagatgaagatgaaaaggaaa |
| | | aagctgcatgttctgctgctgctatggaagaagactcagaagcatcttcct |
| | | caaggataggtgatagctcacagggagacaacaatttgcaaaaattaggcc |
| | | ctgatgatgtgtctgtggatattgatgccattagaagggtctacaccagat |
| | | tgctctctaatgaaaaaattgaaactgcctttctcaatgcacttgtatatt |
| | | tgtcacctaacgtggaatgtgacttgacgtatcacaatgtatactctcgag |
| | | atcctaattatctgaatttgttcattatcgtaatggagaatagaaatctcc |
| | | acagtcctgaatatctggaaatggctttgccattattttgcaaagcgatga |
| | | gcaagctaccccttgcagcccaaggaaaactgatcagactgtggtctaaat |
| | | acaatgcagaccagattcggagaatgatggagacatttcagcaacttatta |
| | | cttataaagtcataagcaatgaatttaacagtcgaaatctagtgaatgatg |
| | | atgatgccattgttgctgcttcgaagtgcttgaaaatggtttactatgcaa |
| | | atgtagtgggaggggaagtggacacaaatcacaatgaagaagatgatgaag |
| | | agcccatccctgagtccagcgagctgacacttcaggaacttttgggagaag |
| | | aaagaagaaacaagaaaggtcctcgagtggaccccctggaaactgaacttg |
| | | gtgttaaaaccctggattgtcgaaaaccacttatcccttttgaagagttta |
| | | ttaatgaaccactgaatgaggttctagaaatggataaagattatacttttt |
| | | tcaaagtagaaacagagaacaaattctcttttatgacatgtccctttatat |
| | | tgaatgctgtcacaaagaatttgggattatattatgacaatagaattcgca |
| | | tgtacagtgaacgaagaatcactgttctctacagcttagttcaaggacagc |
| | | agttgaatccatatttgagactcaaagttagacgtgaccatatcatagatg |
| | | atgcacttgtccggctagagatgatcgctatggaaaatcctgcagacttga |
| | | agaagcagttgtatgtggaatttgaaggagaacaaggagttgatgagggag |
| | | gtgtttccaaagaattttttcagctggttgtggaggaaatcttcaatccag |
| | | atattggtatgttcacatacgatgaatctacaaaattgttttggtttaatc |
| | | catcttcttttgaaactgagggtcagtttactctgattggcatagtactgg |
| | | gtctggctatttacaataactgtatactggatgtacattttcccatggttg |
| | | tctacaggaagctaatggggaaaaaaggaacttttcgtgacttgggagact |
| | | ctcacccagttctatatcagagtttaaaagatttattggagtatgaaggga |
| | | atgtggaagatgacatgatgatcactttccagatatcacagacagatcttt |
| | | ttggtaacccaatgatgtatgatctaaaggaaaatggtgataaaattccaa |
| | | ttacaaatgaaaacaggaaggaatttgtcaatctttattctgactacattc |
| | | tcaataaatcagtagaaaaacagttcaaggcttttcggagaggttttcata |
| | | tggtgaccaatgaatctcccttaaagtacttattcagaccagaagaaattg |
| | | aattgcttatatgtggaagccggaatctagatttccaagcactagaagaaa |
| | | ctacagaatatgacggtggctataccagggactctgttctgattagggagt |
| | | tctgggaaatcgttcattcatttacagatgaacagaaaagactcttcttgc |
| | | agtttacaacgggcacagacagagcacctgtgggaggactaggaaaattaa |
| | | agatgattatagccaaaaatggcccagacacagaaaggttacctacatctc |
| | | atacttgctttaatgtgcttttacttccggaatactcaagcaaagaaaaac |
| | | ttaaagagagattgttgaaggccatcacgtatgccaaaggatttggcatgc |
| | | tgtaagtttaaacaagctttaagatacattgatgagtttggacaaaccaca |
| | | actagaatgcagtgaaaaaaatgctttatttgtgaaatttgtgatgctatt |
| | | gctttatttgtaaccattataagctgcaataaacaagttctcgagccatgg |
| | | gcgcgccatcgatgaggaacccctagtgatggagttggccactccctctct |
| | | gcgcgctcgctcgctcactgaggccgggcgaccaaaggtcgcccgacgccc |
| | | gggctttgcccgggcggcctcagtgagcgagcgagcgcgcagctgcctgca |
| | | ggctgatttaaatcctgcaggcatttaattaagcaagctgtagccaaccac |
| | | tagaactatagctagagtcctgggcgaacaaacgatgctcgccttccagaa |
| | | aaccgaggatgcgaaccacttcatccggggtcagcaccaccggcaagcgcc |
| | | gcgacggccgaggtcttccgatctcctgaagccagggcagatccgtgcaca |
| | | gcaccttgccgtagaagaacagcaaggccgccggccggccaatgcctgacg |
| | | atgcgtggagacctgtacaggcgtaatcatggtcatagctgtttcctgtgt |
| | | gaaattgttatccgctcacaattccacacaacatacgagccggaagcataa |
| | | agtgtaaagcctggggtgcctaatgagtgagctaactcacattaattgcgt |
| | | tgcgctcactgcccgctttccagtcgggaaacctgtcgtgccagctgcatt |
| | | aatgaatcggccaacgcgcggggagaggcggtttgcgtattgggcgctctt |
| | | ccgcttcctcgctcactgactcgctgcgctcggtcgttcggctgcggcgag |
| | | cggtatcagctcactcaaaggcggtaatacggttatccacagaatcagggg |
| | | ataacgcaggaaagaacatgtgagcaaaaggccagcaaaaggccaggaacc |
| | | gtaaaaaggccgcgttgctggcgtttttccataggctccgcccccctgacg |
| | | agcatcacaaaaatcgacgctcaagtcagaggtggcgaaacccgacaggac |
| | | tataaagataccaggcgtttccccctggaagctccctcgtgcgctctcctg |
| | | ttccgaccctgccgcttaccggatacctgtccgcctttctcccttcgggaa |
| | | gcgtggcgctttctcatagctcacgctgtaggtatctcagttcggtgtagg |
| | | tcgttcgctccaagctgggctgtgtgcacgaaccccccgttcagcccgacc |
| | | gctgcgccttatccggtaactatcgtcttgagtccaacccggtaagacacg |
| | | acttatcgccactggcagcagccactggtaacaggattagcagagcgaggt |
| | | atgtaggcggtgctacagagttcttgaagtggtggcctaactacggctaca |
| | | ctagaagaacagtatttggtatctgcgctctgctgaagccagttaccttcg |
| | | gaaaaagagttggtagctcttgatccggcaaacaaaccaccgctggtagcg |
| | | gtggtttttttgtttgcaagcagcagattacgcgcagaaaaaaaggatctc |
| | | aagaagatcctttgatcttttctacggggtctgacgctcagtggaacgaaa |
| | | actcacgttaagggattttggtcatgagattatcaaaaaggatcttcacct |
| | | agatccttttaaattaaaaatgaagttttaaatcaagcccaatctgaataa |
| | | tgttacaaccaattaaccaattctgattagaaaaactcatcgagcatcaaa |
| | | tgaaactgcaatttattcatatcaggattatcaataccatatttttgaaaa |
| | | agccgtttctgtaatgaaggagaaaactcaccgaggcagttccataggatg |
| | | gcaagatcctggtatcggtctgcgattccgactcgtccaacatcaatacaa |
| | | cctattaatttcccctcgtcaaaaataaggttatcaagtgagaaatcacca |
| | | tgagtgacgactgaatccggtgagaatggcaaaagtttatgcatttctttc |
| | | cagacttgttcaacaggccagccattacgctcgtcatcaaaatcactcgca |
| | | tcaaccaaaccgttattcattcgtgattgcgcctgagcgagacgaaatacg |
| | | cgatcgctgttaaaaggacaattacaaacaggaatcgaatgcaaccggcgc |
| | | aggaacactgccagcgcatcaacaatattttcacctgaatcaggatattct |
| | | tctaatacctggaatgctgtttttccggggatcgcagtggtgagtaaccat |
| | | gcatcatcaggagtacggataaaatgcttgatggtcggaagaggcataaat |
| | | tccgtcagccagtttagtctgaccatctcatctgtaacatcattggcaacg |
| | | ctacctttgccatgtttcagaaacaactctggcgcatcgggcttcccatac |
| | | aagcgatagattgtcgcacctgattgcccgacattatcgcgagcccattta |
| | | tacccatataaatcagcatccatgttggaatttaatcgcggcctcgacgtt |
| | | tcccgttgaatatggctcataacaccccttgtattactgtttatgtaagca |
| | | gacagttttattgttcatgatgatatatttttatcttgtgcaatgtaacat |
| | | cagagattttgagacacgggccagagctgca |
| 23 | P-T178 sequence | tcgcgcgtttcggtgatgacggtgaaaacctctgacacatgcagctcccgg |
| | | agacggtcacagcttgtctgtaagcggatgccgggagcagacaagcccgtc |
| | | agggcgcgtcagcgggtgttggcgggtgtcggggctggcttaactatgcgg |
| | | catcagagcagattgtactgagagtgcacgatatcgggtccccaattgaca |
| | | ttattgaagcatttatcagggttattgtctcagaatttaaatgacgcctgc |
| | | aggcagctgcgcgctcgctcgctcactgaggccgcccgggcaaagcccggg |
| | | cgtcgggcgacctttggtcgcccggcctcagtgagcgagcgagcgcgcaga |
| | | gagggagtggccaactccatcactaggggttccttgcgtcgacACACACAC |
| | | ACCCACACACTTGGCTATACAAAAAAGATGTTCCGAGACTGACAGTTAAAA |
| | | ATTACACTGCTGGCCGAGCACTGTGGCTCATGCCTGTAGTCCCAAAACTTT |
| | | GGGAGGCCGAGGTGGGAGGATCACTTGAGGCCAGGAGCTCGAGACCAGCCT |
| | | GGGCCTAACATAGCAAGACACCATTTCTAAGTTTAAAAATAAAAATAAATT |
| | | TAAAAAAGATACACTGCTATGTACGACTGTTGATATAAAAAATCTGAATAC |
| | | TAGACATGGGTACTCATCTAGTACTTCAAGGGCTTATCAACAAAGTTGCAA |
| | | GTTGTAACACTATGAATTGTTAGTGATACTCTTTTGGCTTTGCTAGCAAGT |
| | | GTTGTAAAGCTATACACACACACACACACACACACACACATACACACACAC |
| | | ACCTTTTAAAATGGTGACCCTGGTACCAAATATGACTTTAAATGGATTTAA |
| | | TTTTAATGGCTTTAACTACGTTCAGCTGTCATATGGATCAAAATTAGCCTC |
| | | TATCCAGCTGGGGTCAACCAGGGAGCCACTTTTCTTAACCGACGACCTACT |
| | | GAACGTCAACAACTGCAGGAGACGGGACTTTACCTTCGTCTCTGGTAAACT |
| | | AGTTGACACATCCTGTGTTGGCAAGAGGCCTAAGTAGATGACCTTGGTCCT |
| | | CTAAAATCTGGCCTGCACTCTCGGGGCACCCCTGCAACATCTACAAAGGCA |
| | | GCTCCAGATAGAAAAGGGTTGGGGTCGAAAAGCCAATAACGGCAGGCACCT |
| | | GCCCCGCCTCGGGGCTGGGGGGCTATTCCAGCGGCTTCAGCTAACTTTCAG |
| | | AGCCATTCGTTTCCCAACAAAGTCTGAGGCGTTCCTCTGCTGGGTACACCA |
| | | AGGGGCTCTGCAACCCTCCTGGGGGGGGGGGTGCCCAGAGGGCTTCCGGAA |
| | | GTCCCAGGTTTATTCTTTCGGGTCACAGACAGCAGAAACTAAAAAGAGGGA |
| | | TTACCCTTTCTGTCCAGTCGCAAGATGGCGACCGAGCCTGGTGGGACTCCG |
| | | AGGGGCCGCAGGCCACCTCCTCTTCCCAATGGCCCGTGCGCCGGCGGCGAC |
| | | GGCAAGCGGGAGGGAGGCGGGGCCGGCGAAGGAAGGAGGGGCGGAGCGCGG |
| | | CGCCCTCCCGCGCGTCTTGGCCCCGCCCCACGTCCCCGCGTCCCGGCCTGG |
| | | AGCCCTCGCCCGGCCGGGCGGCGCGCGCTGCCTGCCGGGATACTCGGCCCG |
| | | CCCcaCCGGTatgcatgtaagtttagtctttttgtcttttatttcaggtcc |
| | | cggatccggtggtggtgcaaatcaaagaactgctcctcagtggatgttgcc |
| | | tttacttctagcaccggtcgccaccatgaagcgagcagctgcaaagcatct |
| | | aatagaacgctactaccaccagttaactgagggctgtggaaatgaagcctg |
| | | cacgaatgagttttgtgcttcctgtccaacttttcttcgtatggataataa |
| | | tgcagcagctattaaagccctcgagctttataagattaatgcaaaactctg |
| | | tgatcctcatccctccaagaaaggagcaagctcagcttaccttgagaactc |
| | | gaaaggtgcccccaacaactcctgctctgagataaaaatgaacaagaaagg |
| | | cgctagaattgattttaaagatgtgacttacttaacagaagagaaggtata |
| | | tgaaattcttgaattatgtagagaaagagaggattattcccctttaatccg |
| | | tgttattggaagagttttttctagtgctgaggcattggtacagagcttccg |
| | | gaaagttaaacaacacaccaaggaagaactgaaatctcttcaagcaaaaga |
| | | tgaagacaaagatgaagatgaaaaggaaaaagctgcatgttctgctgctgc |
| | | tatggaagaagactcagaagcatcttcctcaaggataggtgatagctcaca |
| | | gggagacaacaatttgcaaaaattaggccctgatgatgtgtctgtggatat |
| | | tgatgccattagaagggtctacaccagattgctctctaatgaaaaaattga |
| | | aactgcctttctcaatgcacttgtatatttgtcacctaacgtggaatgtga |
| | | cttgacgtatcacaatgtatactctcgagatcctaattatctgaatttgtt |
| | | cattatcgtaatggagaatagaaatctccacagtcctgaatatctggaaat |
| | | ggctttgccattattttgcaaagcgatgagcaagctaccccttgcagccca |
| | | aggaaaactgatcagactgtggtctaaatacaatgcagaccagattcggag |
| | | aatgatggagacatttcagcaacttattacttataaagtcataagcaatga |
| | | atttaacagtcgaaatctagtgaatgatgatgatgccattgttgctgcttc |
| | | gaagtgcttgaaaatggtttactatgcaaatgtagtgggaggggaagtgga |
| | | cacaaatcacaatgaagaagatgatgaagagcccatccctgagtccagcga |
| | | gctgacacttcaggaacttttgggagaagaaagaagaaacaagaaaggtcc |
| | | tcgagtggaccccctggaaactgaacttggtgttaaaaccctggattgtcg |
| | | aaaaccacttatcccttttgaagagtttattaatgaaccactgaatgaggt |
| | | tctagaaatggataaagattatacttttttcaaagtagaaacagagaacaa |
| | | attctcttttatgacatgtccctttatattgaatgctgtcacaaagaattt |
| | | gggattatattatgacaatagaattcgcatgtacagtgaacgaagaatcac |
| | | tgttctctacagcttagttcaaggacagcagttgaatccatatttgagact |
| | | caaagttagacgtgaccatatcatagatgatgcacttgtccggctagagat |
| | | gatcgctatggaaaatcctgcagacttgaagaagcagttgtatgtggaatt |
| | | tgaaggagaacaaggagttgatgagggaggtgtttccaaagaattttttca |
| | | gctggttgtggaggaaatcttcaatccagatattggtatgttcacatacga |
| | | tgaatctacaaaattgttttggtttaatccatcttcttttgaaactgaggg |
| | | tcagtttactctgattggcatagtactgggtctggctatttacaataactg |
| | | tatactggatgtacattttcccatggttgtctacaggaagctaatggggaa |
| | | aaaaggaacttttcgtgacttgggagactctcacccagttctatatcagag |
| | | tttaaaagatttattggagtatgaagggaatgtggaagatgacatgatgat |
| | | cactttccagatatcacagacagatctttttggtaacccaatgatgtatga |
| | | tctaaaggaaaatggtgataaaattccaattacaaatgaaaacaggaagga |
| | | atttgtcaatctttattctgactacattctcaataaatcagtagaaaaaca |
| | | gttcaaggcttttcggagaggttttcatatggtgaccaatgaatctccctt |
| | | aaagtacttattcagaccagaagaaattgaattgcttatatgtggaagccg |
| | | gaatctagatttccaagcactagaagaaactacagaatatgacggtggcta |
| | | taccagggactctgttctgattagggagttctgggaaatcgttcattcatt |
| | | tacagatgaacagaaaagactcttcttgcagtttacaacgggcacagacag |
| | | agcacctgtgggaggactaggaaaattaaagatgattatagccaaaaatgg |
| | | cccagacacagaaaggttacctacatctcatacttgctttaatgtgctttt |
| | | acttccggaatactcaagcaaagaaaaacttaaagagagattgttgaaggc |
| | | catcacgtatgccaaaggatttggcatgctgtaagtttaaacaagctttaa |
| | | gatacattgatgagtttggacaaaccacaactagaatgcagtgaaaaaaat |
| | | gctttatttgtgaaatttgtgatgctattgctttatttgtaaccattataa |
| | | gctgcaataaacaagttctcgagccatgggcgcgccatcgatgaggaaccc |
| | | ctagtgatggagttggccactccctctctgcgcgctcgctcgctcactgag |
| | | gccgggcgaccaaaggtcgcccgacgcccgggctttgcccgggcggcctca |
| | | gtgagcgagcgagcgcgcagctgcctgcaggctgatttaaatcctgcaggc |
| | | atttaattaagcaagctgtagccaaccactagaactatagctagagtcctg |
| | | ggcgaacaaacgatgctcgccttccagaaaaccgaggatgcgaaccacttc |
| | | atccggggtcagcaccaccggcaagcgccgcgacggccgaggtcttccgat |
| | | ctcctgaagccagggcagatccgtgcacagcaccttgccgtagaagaacag |
| | | caaggccgccggccggccaatgcctgacgatgcgtggagacctgtacaggc |
| | | gtaatcatggtcatagctgtttcctgtgtgaaattgttatccgctcacaat |
| | | tccacacaacatacgagccggaagcataaagtgtaaagcctggggtgccta |
| | | atgagtgagctaactcacattaattgcgttgcgctcactgcccgctttcca |
| | | gtcgggaaacctgtcgtgccagctgcattaatgaatcggccaacgcgcggg |
| | | gagaggcggtttgcgtattgggcgctcttccgcttcctcgctcactgactc |
| | | gctgcgctcggtcgttcggctgcggcgagcggtatcagctcactcaaaggc |
| | | ggtaatacggttatccacagaatcaggggataacgcaggaaagaacatgtg |
| | | agcaaaaggccagcaaaaggccaggaaccgtaaaaaggccgcgttgctggc |
| | | gtttttccataggctccgcccccctgacgagcatcacaaaaatcgacgctc |
| | | aagtcagaggtggcgaaacccgacaggactataaagataccaggcgtttcc |
| | | ccctggaagctccctcgtgcgctctcctgttccgaccctgccgcttaccgg |
| | | atacctgtccgcctttctcccttcgggaagcgtggcgctttctcatagctc |
| | | acgctgtaggtatctcagttcggtgtaggtcgttcgctccaagctgggctg |
| | | tgtgcacgaaccccccgttcagcccgaccgctgcgccttatccggtaacta |
| | | tcgtcttgagtccaacccggtaagacacgacttatcgccactggcagcagc |
| | | cactggtaacaggattagcagagcgaggtatgtaggcggtgctacagagtt |
| | | cttgaagtggtggcctaactacggctacactagaagaacagtatttggtat |
| | | ctgcgctctgctgaagccagttaccttcggaaaaagagttggtagctcttg |
| | | atccggcaaacaaaccaccgctggtagcggtggtttttttgtttgcaagca |
| | | gcagattacgcgcagaaaaaaaggatctcaagaagatcctttgatcttttc |
| | | tacggggtctgacgctcagtggaacgaaaactcacgttaagggattttggt |
| | | catgagattatcaaaaaggatcttcacctagatccttttaaattaaaaatg |
| | | aagttttaaatcaagcccaatctgaataatgttacaaccaattaaccaatt |
| | | ctgattagaaaaactcatcgagcatcaaatgaaactgcaatttattcatat |
| | | caggattatcaataccatatttttgaaaaagccgtttctgtaatgaaggag |
| | | aaaactcaccgaggcagttccataggatggcaagatcctggtatcggtctg |
| | | cgattccgactcgtccaacatcaatacaacctattaatttcccctcgtcaa |
| | | aaataaggttatcaagtgagaaatcaccatgagtgacgactgaatccggtg |
| | | agaatggcaaaagtttatgcatttctttccagacttgttcaacaggccagc |
| | | cattacgctcgtcatcaaaatcactcgcatcaaccaaaccgttattcattc |
| | | gtgattgcgcctgagcgagacgaaatacgcgatcgctgttaaaaggacaat |
| | | tacaaacaggaatcgaatgcaaccggcgcaggaacactgccagcgcatcaa |
| | | caatattttcacctgaatcaggatattcttctaatacctggaatgctgttt |
| | | ttccggggatcgcagtggtgagtaaccatgcatcatcaggagtacggataa |
| | | aatgcttgatggtcggaagaggcataaattccgtcagccagtttagtctga |
| | | ccatctcatctgtaacatcattggcaacgctacctttgccatgtttcagaa |
| | | acaactctggcgcatcgggcttcccatacaagcgatagattgtcgcacctg |
| | | attgcccgacattatcgcgagcccatttatacccatataaatcagcatcca |
| | | tgttggaatttaatcgcggcctcgacgtttcccgttgaatatggctcataa |
| | | caccccttgtattactgtttatgtaagcagacagttttattgttcatgatg |
| | | atatatttttatcttgtgcaatgtaacatcagagattttgagacacgggcc |
| | | agagctgca |
| 24 | hP1 promoter | ACACACACACCCACACACTTGGCTATACAAAAAAGATGTTCCGAGACTGAC |
| | | AGTTAAAAATTACACTGCTGGCCGAGCACTGTGGCTCATGCCTGTAGTCCC |
| | | AAAACTTTGGGAGGCCGAGGTGGGAGGATCACTTGAGGCCAGGAGCTCGAG |
| | | ACCAGCCTGGGCCTAACATAGCAAGACACCATTTCTAAGTTTAAAAATAAA |
| | | AATAAATTTAAAAAAGATACACTGCTATGTACGACTGTTGATATAAAAAAT |
| | | CTGAATACTAGACATGGGTACTCATCTAGTACTTCAAGGGCTTATCAACAA |
| | | AGTTGCAAGTTGTAACACTATGAATTGTTAGTGATACTCTTTTGGCTTTGC |
| | | TAGCAAGTGTTGTAAAGCTATACACACACACACACACACACACACACATAC |
| | | ACACACACACCTTTTAAAATGGTGACCCTGGTACCAAATATGACTTTAAAT |
| | | GGATTTAATTTTAATGGCTTTAACTACGTTCAGCTGTCATATGGATCAAAA |
| | | TTAGCCTCTATCCAGCTGGGGTCAACCAGGGAGCCACTTTTCTTAACCGAC |
| | | GACCTACTGAACGTCAACAACTGCAGGAGACGGGACTTTACCTTCGTCTCT |
| | | GGTAAACTAGTTGACACATCCTGTGTTGGCAAGAGGCCTAAGTAGATGACC |
| | | TTGGTCCTCTAAAATCTGGCCTGCACTCTCGGGGCACCCCTGCAACATCTA |
| | | CAAAGGCAGCTCCAGATAGAAAAGGGTTGGGGTCGAAAAGCCAATAACGGC |
| | | AGGCACCTGCCCCGCCTCGGGGCTGGGGGGCTATTCCAGCGGCTTCAGCTA |
| | | ACTTTCAGAGCCATTCGTTTCCCAACAAAGTCTGAGGCGTTCCTCTGCTGG |
| | | GTACACCAAGGGGCTCTGCAACCCTCCTGGGGGGGGGGGTGCCCAGAGGGC |
| | | TTCCGGAAGTCCCAGGTTTATTCTTTCGGGTCACAGACAGCAGAAACTAAA |
| | | AAGAGGGATTACCCTTTCTGTCCAGTCGCAAGATGGCGACCGAGCCTGGTG |
| | | GGACTCCGAGGGGCCGCAGGCCACCTCCTCTTCCCAATGGCCCGTGCGCCG |
| | | GCGGCGACGGCAAGCGGGAGGGAGGCGGGGCCGGCGAAGGAAGGAGGGGCG |
| | | GAGCGCGGCGCCCTCCCGCGCGTCTTGGCCCCGCCCCACGTCCCCGCGTCC |
| | | CGGCCTGGAGCCCTCGCCCGGCCGGGCGGCGCGCGCTGCCTGCCGGGATAC |
| | | TCGGCCCGCCC |

### NUMBERED EMBODIMENTS

The following list of embodiments is included herein for illustration purposes only and is not intended to be comprehensive or limiting. The subject matter to be claimed is expressly not limited to the following embodiments.
Embodiment 1. A nucleic acid molecule, comprising an adeno-associated virus (AAV) expression cassette, wherein the AAV expression cassette comprises, from 5′ to 3′:
   (i) a 5′ AAV inverted terminal repeat (ITR);
   (ii) a promoter;
   (iii) an Angelman syndrome-associated transgene; and
   (iv) a 3′ AAV ITR.
Embodiment 2. The nucleic acid molecule of embodiment 1, wherein the promoter drives expression of the Angelman syndrome-associated transgene.
Embodiment 3. The nucleic acid molecule of embodiment 1 or 2, wherein the promoter is capable of expressing the transgene in a neuronal cell.
Embodiment 4. The nucleic acid molecule of any one of embodiments 1-3, wherein the promoter comprises a synapsin (SYN) promoter.
Embodiment 5. The nucleic acid molecule of embodiment 4, wherein the SYN promoter comprises a nucleic acid sequence derived from: (i) a human SYN promoter, (ii) a chicken SYN promoter, (iii) a mouse SYN promoter, or (iv) any combination thereof.
Embodiment 6. The nucleic acid molecule of embodiment 5, wherein the SYN promoter comprises a human SYN (hSYN) promoter.
Embodiment 7. The nucleic acid molecule of any one of embodiments 4-6, wherein the hSYN promoter comprises the nucleic acid sequence SEQ ID NO: 3, or a sequence at least 90% identical thereto.
Embodiment 8. The nucleic acid molecule of any one of embodiments 1-7, wherein the Angelman syndrome-associated transgene encodes a ubiquitin protein ligase E3A (UBE3A).
Embodiment 9. The nucleic acid molecule of any one of embodiments 1-8, wherein the Angelman syndrome-associated transgene encodes a human UBE3A (hUBE3A).
Embodiment 10. The nucleic acid molecule of embodiment 8 or 9, wherein the Angelman syndrome-associated transgene comprises a mutation capable of removing a predicted cryptic splice site.
Embodiment 11. The nucleic acid molecule of embodiment 10, wherein the Angelman syndrome-associated transgene comprises a nucleic acid substitution of G2556C, relative to the nucleic acid sequence of wild type human UBE3A gene.
Embodiment 12. The nucleic acid molecule of embodiment 11, wherein the Angelman syndrome-associated transgene comprises a nucleic acid sequence having at least 90% identity of SEQ ID NO: 12, and a nucleic acid substitution of G2556C, relative to SEQ ID NO: 12.
Embodiment 13. The nucleic acid molecule of any one of embodiments 1-12, wherein the Angelman syndrome-associated transgene comprises a nucleic acid sequence having at least 90% identity to SEQ ID NO: 5.
Embodiment 14. The nucleic acid molecule of any one of embodiments 1-13, wherein the Angelman syndrome-associated transgene comprises a nucleic acid sequence having at least 90% identity of SEQ ID NO: 5, and a nucleic acid substitution of G2556C, relative to SEQ ID NO: 12.
Embodiment 15. The nucleic acid molecule of any one of embodiments 1-14, wherein at least one of the 5′ ITR and the 3′ ITR is about 110 to about 160 nucleotides in length. Embodiment 16. The nucleic acid molecule of any one of embodiments 1-15, wherein the 5′ ITR is the same length as the 3′ ITR.
Embodiment 17. The nucleic acid molecule of any one of embodiments 1-16, wherein the 5′ ITR and the 3′ ITR are each about 145 nucleotides in length.
Embodiment 18. The nucleic acid molecule of any one of embodiments 1-16, wherein the 5′ ITR and the 3′ ITR are each about 141 nucleotides in length.
Embodiment 19. The nucleic acid molecule of any one of embodiments 1-18, wherein at least one of the 5′ ITR and the 3′ ITR is isolated or derived from the genome of AAV1, AAV2, AAV3, AAV4, AAV5, AAV6, AAV7, AAV8, AAV9, AAV10, AAV11, AAV12, AAVrh8, AAVrh10, AAVrh32.33, AAVrh74, Avian AAV or Bovine AAV.
Embodiment 20. The nucleic acid molecule of any one of embodiments 1-19, wherein the 5′ ITR and the 3′ ITR are each isolated or derived from the genome of AAV2.
Embodiment 21. The nucleic acid molecule of any one of embodiments 1-20, wherein the 5′ ITR comprises the sequence of SEQ ID NO: 2 or SEQ ID NO: 9.
Embodiment 22. The nucleic acid molecule of any one of embodiments 1-21, wherein the 3′ ITR comprises the sequence of SEQ ID NO: 8 or SEQ ID NO: 10.
Embodiment 23. The nucleic acid molecule of any one of embodiments 1-22, wherein the AAV expression cassette comprises an intron.
Embodiment 24. The nucleic acid molecule of embodiment 23, wherein the intron is derived from the human beta-globin gene (hBGIN).
Embodiment 25. The nucleic acid molecule of embodiment 24, wherein the intron comprises one or more of the following mutations relative to SEQ ID NO: 13: (i) mutation at the 5′ terminus to contain Exon 2 splicing donor (AGG), (ii) mutation at the 3′ terminus to contain Exon 3 splicing acceptor (CTC), and (iii) G74T and G205A.
Embodiment 26. The nucleic acid molecule of embodiment 24 or embodiment 25, wherein the intron comprises a nucleic acid sequence of SEQ ID NO: 4, or a sequence at least 90% identical thereto.
Embodiment 27. The nucleic acid molecule of any one of embodiments 1-26, wherein the AAV expression cassette comprises a polyadenylation signal.
Embodiment 28. The nucleic acid molecule of embodiment 27, wherein the polyadenylation signal is a polyadenylation signal isolated or derived from one or more of the following genes: simian virus 40 (SV40), rBG, α-globin, β-globin, human collagen, human growth hormone (hGH), polyoma virus, human growth hormone (hGH) or bovine growth hormone (bGH).
Embodiment 29. The nucleic acid molecule of embodiment 27 or embodiment 28, wherein the AAV expression cassette comprises a bGH polyadenylation signal.
Embodiment 30. The nucleic acid molecule of embodiment 29, wherein the bGH polyadenylation signal comprises a nucleic acid sequence of SEQ ID NO: 6, or a sequence at least 90% identical thereto.
Embodiment 31. The nucleic acid molecule of any one of embodiments 1-30, wherein the AAV expression cassette comprises at least one stuffer sequence.
Embodiment 32. The nucleic acid molecule of embodiment 31, wherein the at least one stuffer sequence comprises a nucleic acid sequence of SEQ ID NO: 7, or a sequence at least 90% identical thereto.
Embodiment 33. The nucleic acid molecule of any one of embodiments 1-32, wherein the AAV expression cassette comprises a Kozak sequence.
Embodiment 34. The nucleic acid molecule of embodiment 33, wherein the Kozak sequence comprises the nucleic acid sequence of SEQ ID NO: 14, or a sequence at least 90% identical thereto; or the nucleic acid sequence of acagccacc, or a sequence at least 90% identical thereto.
Embodiment 35. The nucleic acid molecule of any one of embodiments 1-34, wherein the AAV expression cassette comprises an enhancer.
Embodiment 36. The nucleic acid molecule of any one of embodiments 1-35, wherein the AAV expression cassette comprises a nucleic acid sequence SEQ ID NO: 1, or a sequence at least 90% identical thereto.
Embodiment 37. The nucleic acid molecule of any one of embodiments 1-35, wherein the AAV expression cassette comprises a nucleic acid sequence SEQ ID NO: 11, or a sequence at least 90% identical thereto.
Embodiment 38. A plasmid, comprising the nucleic acid molecule of any one of embodiments 1-37.
Embodiment 39. A cell, comprising the nucleic acid molecule of any one of embodiments 1-37 or the plasmid of embodiment 38.
Embodiment 40. A method of producing a recombinant AAV vector, the method comprising contacting an AAV producer cell with the nucleic acid molecule of any one of embodiments 1-37 or the plasmid of embodiment 38.
Embodiment 41. A recombinant AAV vector produced by the method of embodiment 40.
Embodiment 42. The recombinant AAV vector of embodiment 41, wherein the vector is of a serotype selected from AAV1, AAV2, AAV3, AAV4, AAV5, AAV6, AAV7, AAV8, AAV9, AAV10, AAV11, AAV12, AAVrh8, AAVrh10, AAVrh32.33, AAVrh74, Avian AAV and Bovine AAV.
Embodiment 43. The recombinant AAV vector of embodiment 41 or embodiment 42, wherein the recombinant AAV vector is a single-stranded AAV (ssAAV).
Embodiment 44. The recombinant AAV vector of embodiment 41 or embodiment 42, wherein the recombinant AAV vector is a self-complementary AAV (scAAV).
Embodiment 45. The recombinant AAV vector of any one of embodiments 41-44, wherein the AAV vector comprises a capsid protein of AAV1, AAV2, AAV3, AAV4, AAV5, AAV6, AAV7, AAV8, AAV9, AAV10, AAV11, AAV12, AAVrh8, AAVrh10, AAVrh32.33, AAVrh74, Avian AAV or Bovine AAV.
Embodiment 46. The recombinant AAV vector of any one of embodiments 41-45, wherein the AAV vector comprises a capsid protein with one or more substitutions or mutations, as compared to a wild type AAV capsid protein.
Embodiment 47. The recombinant AAV vector of any one of embodiments 41-46, wherein the AAV vector comprises a capsid protein comprising:
(i) (i) the amino acid sequence of SEQ ID NO: 15, or a sequence at least 90% identical thereto, or
(ii) (ii) the amino acid sequence of SEQ ID NO: 16, or a sequence at least 90% identical thereto, or
(iii) (iii) the amino acid sequence of SEQ ID NO: 17, or a sequence at least 90% identical thereto.
Embodiment 48. The recombinant AAV vector of embodiment 47, wherein the AAV vector comprises a capsid protein comprising the amino acid sequence of SEQ ID NO: 15, or a sequence at least 90% identical thereto.
Embodiment 49. The recombinant AAV vector of embodiment 48, wherein the AAV vector comprises a capsid protein comprising the amino acid sequence of SEQ ID NO: 15.
Embodiment 50. The recombinant AAV vector of embodiment 47, wherein the AAV vector comprises a capsid protein comprising the amino acid sequence of SEQ ID NO: 16, or a sequence at least 90% identical thereto.
Embodiment 51. The recombinant AAV vector of embodiment 50, wherein the AAV vector comprises a capsid protein comprising the amino acid sequence of SEQ ID NO: 16.
Embodiment 52. The recombinant AAV vector of embodiment 47, wherein the AAV vector comprises a capsid protein comprising the amino acid sequence of SEQ ID NO: 17, or a sequence at least 90% identical thereto.
Embodiment 53. The recombinant AAV vector of embodiment 52, wherein the AAV vector comprises a capsid protein comprising the amino acid sequence of SEQ ID NO: 17.
Embodiment 54. A composition, comprising: (a) the nucleic acid molecule of any one of embodiments 1-37, the plasmid of embodiment 38, the cell of embodiment 39, or the recombinant AAV vector of any one of embodiments 41-53; and (b) a pharmaceutically acceptable carrier.
Embodiment 55. A method of expressing an Angelman syndrome-associated transgene in a tissue, comprising: contacting the tissue with the nucleic acid molecule of any one of embodiments 1-37, the plasmid of embodiment 38, the recombinant AAV vector of any one of embodiments 41-53, or the composition of embodiment 54, thereby expressing the Angelman syndrome-associated transgene in the tissue.
Embodiment 56. The method of embodiment 55, wherein the tissue comprises brain tissue.
Embodiment 57. The method of embodiment 55 or embodiment 56, wherein the tissue comprises neuronal cells.
Embodiment 58. The method of any one of embodiments 55-57, wherein the contacting step is performed in vitro, ex vivo, or in vivo.
Embodiment 59. The method of embodiment 58, wherein the contacting step is performed in vivo in a subject in need thereof.
Embodiment 60. The method of embodiment 59, wherein the contacting step comprises administering a therapeutically effective amount of the nucleic acid molecule, the plasmid, the recombinant AAV vector, or the composition to the subject.
Embodiment 61. The method of embodiment 59 or embodiment 60, wherein the subject suffers from, or is at a risk of developing, the Angelman syndrome.
Embodiment 62. A method for treating Angelman syndrome in a subject in need thereof, comprising administering to the subject a therapeutically effective amount of the nucleic acid molecule of any one of embodiments 1-37, the plasmid of embodiment 38, the cell of embodiment 39, the recombinant AAV vector of any one of embodiments 41-53, or the composition of embodiment 54, thereby treating Angelman syndrome in the subject.
Embodiment 63. The method of embodiment 62, wherein the subject suffers from, or is at a risk of developing, the Angelman syndrome.
Embodiment 64. The method of any one of embodiments 61-63, wherein the Angelman syndrome is associated with, promoted by, or caused by a genetic mutation.
Embodiment 65. The method of embodiment 64, wherein the genetic mutation comprises a mutation in the human UBE3A gene.
Embodiment 66. The method of embodiment 64, wherein the genetic mutation comprises a mutation in the chromosomal region 15q11-q13.
Embodiment 67. The method of any one of embodiments 61-66, wherein the method comprises diminishing the severity of; delaying the onset or progression of; and/or eliminating a symptom of the Angelman syndrome.
Embodiment 68. The method of embodiment 67, wherein the symptom of the Angelman syndrome comprises: (a) developmental delay, (b) intellectual disability, (c) speech impairment, (d) gait ataxia, (e) tremulousness of the limbs, (f) frequent laughing or smiling, (g) excitability, (h) microcephaly, (i) seizures, (j) trouble sleeping, (k) tongue thrusting, (l) hand flapping, (m) curved spine or (n) any combination thereof.
Embodiment 69. The method of any one of embodiments 61-68, wherein the method comprises prolonging the survival of the subject, as compared to a control subject having Angelman syndrome, wherein the control subject has not been administered the therapeutically effective amount, or as compared to the expected survival of the subject prior to administration of the therapeutically effective amount.
Embodiment 70. The method of any one of embodiment 60-69, wherein the subject is a human subject.

What is claimed is:

1. A nucleic acid molecule, comprising an adeno-associated virus (AAV) expression cassette, wherein the AAV expression cassette comprises, from 5′ to 3′:
(i) a 5′ AAV inverted terminal repeat (ITR);
(ii) a promoter;
(iii) an Angelman syndrome-associated transgene; and
(iv) a 3′ AAV ITR.

2. The nucleic acid molecule of claim 1, wherein the promoter drives expression of the Angelman syndrome-associated transgene.

3. The nucleic acid molecule of claim 1 or 2, wherein the promoter drives expression of the transgene in a neuronal cell.

4. The nucleic acid molecule of any one of claims 1-3, wherein the promoter comprises a synapsin (SYN) promoter.

5. The nucleic acid molecule of claim 4, wherein the SYN promoter comprises a nucleic acid sequence derived from: (i) a human SYN promoter, (ii) a chicken SYN promoter, (iii) a mouse SYN promoter, or (iv) any combination thereof.

6. The nucleic acid molecule of claim 5, wherein the SYN promoter comprises a human SYN (hSYN) promoter.

7. The nucleic acid molecule of any one of claims 4-6, wherein the hSYN promoter comprises the nucleic acid sequence SEQ ID NO: 3, or a sequence at least 90% identical thereto.

8. The nucleic acid molecule of any one of claims 1-7, wherein the Angelman syndrome-associated transgene encodes a ubiquitin protein ligase E3A (UBE3A).

9. The nucleic acid molecule of any one of claims 1-8, wherein the Angelman syndrome-associated transgene encodes a human UBE3A (hUBE3A).

10. The nucleic acid molecule of claim 8 or 9, wherein the Angelman syndrome-associated transgene comprises a mutation capable of removing a predicted cryptic splice site.

11. The nucleic acid molecule of claim 10, wherein the Angelman syndrome-associated transgene comprises a nucleic acid substitution of G2556C, relative to the nucleic acid sequence of wild type human UBE3A gene.

12. The nucleic acid molecule of claim 11, wherein the Angelman syndrome-associated transgene comprises a nucleic acid sequence having at least 90% identity of SEQ ID NO: 12, and a nucleic acid substitution of G2556C, relative to SEQ ID NO: 12.

13. The nucleic acid molecule of any one of claims 1-12, wherein the Angelman syndrome-associated transgene comprises a nucleic acid sequence having at least 90% identity to SEQ ID NO: 5.

14. The nucleic acid molecule of any one of claims 1-13, wherein the Angelman syndrome-associated transgene comprises a nucleic acid sequence having at least 90% identity of SEQ ID NO: 5, and a nucleic acid substitution of G2556C, relative to SEQ ID NO: 12.

15. The nucleic acid molecule of any one of claims 1-14, wherein at least one of the 5′ ITR and the 3′ ITR is about 110 to about 160 nucleotides in length.

16. The nucleic acid molecule of any one of claims 1-15, wherein the 5′ ITR is the same length as the 3′ ITR.

17. The nucleic acid molecule of any one of claims 1-16, wherein the 5′ ITR and the 3′ ITR are each about 145 nucleotides in length.

18. The nucleic acid molecule of any one of claims 1-16, wherein the 5′ ITR and the 3′ ITR are each about 141 nucleotides in length.

19. The nucleic acid molecule of any one of claims 1-18, wherein at least one of the 5′ ITR and the 3′ ITR is isolated or derived from the genome of AAV1, AAV2, AAV3, AAV4, AAV5, AAV6, AAV7, AAV8, AAV10, AAV11, AAV12, AAVrh8, AAVrh10, AAVrh32.33, AAVrh74, Avian AAV or Bovine AAV.

20. The nucleic acid molecule of any one of claims 1-19, wherein the 5′ ITR and the 3′ ITR are each isolated or derived from the genome of AAV2.

21. The nucleic acid molecule of any one of claims 1-20, wherein the 5′ ITR comprises the sequence of SEQ ID NO: 2 or SEQ ID NO: 9.

22. The nucleic acid molecule of any one of claims 1-21, wherein the 3′ ITR comprises the sequence of SEQ ID NO: 8 or SEQ ID NO: 10.

23. The nucleic acid molecule of any one of claims 1-22, wherein the AAV expression cassette comprises an intron.

24. The nucleic acid molecule of claim 23, wherein the intron is derived from the human beta-globin gene (hBGIN).

25. The nucleic acid molecule of claim 24, wherein the intron comprises one or more of the following mutations relative to SEQ ID NO: 13: (i) mutation at the 5′ terminus to contain Exon 2 splicing donor (AGG), (ii) mutation at the 3′ terminus to contain Exon 3 splicing acceptor (CTC), and (iii) G74T and G205A.

26. The nucleic acid molecule of claim 24 or claim 25, wherein the intron comprises a nucleic acid sequence of SEQ ID NO: 4, or a sequence at least 90% identical thereto.

27. The nucleic acid molecule of any one of claims 1-26, wherein the AAV expression cassette comprises a polyadenylation signal.

28. The nucleic acid molecule of claim 27, wherein the polyadenylation signal is a polyadenylation signal isolated or derived from one or more of the following genes: simian virus 40 (SV40), rBG, α-globin, β-globin, human collagen, human growth hormone (hGH), polyoma virus, human growth hormone (hGH) or bovine growth hormone (bGH).

29. The nucleic acid molecule of claim 27 or claim 28, wherein the AAV expression cassette comprises a bGH polyadenylation signal.

30. The nucleic acid molecule of claim 29, wherein the bGH polyadenylation signal comprises a nucleic acid sequence of SEQ ID NO: 6, or a sequence at least 90% identical thereto.

31. The nucleic acid molecule of any one of claims 1-30, wherein the AAV expression cassette comprises at least one stuffer sequence.

32. The nucleic acid molecule of claim 31, wherein the at least one stuffer sequence comprises a nucleic acid sequence of SEQ ID NO: 7, or a sequence at least 90% identical thereto.

33. The nucleic acid molecule of any one of claims 1-32, wherein the AAV expression cassette comprises a Kozak sequence.

34. The nucleic acid molecule of claim 33, wherein the Kozak sequence comprises the nucleic acid sequence of SEQ ID NO: 14, or a sequence at least 90% identical thereto; or the nucleic acid sequence of acagccacc, or a sequence at least 90% identical thereto.

35. The nucleic acid molecule of any one of claims 1-34, wherein the AAV expression cassette comprises an enhancer.

36. The nucleic acid molecule of any one of claims 1-35, wherein the AAV expression cassette comprises a nucleic acid sequence SEQ ID NO: 1, or a sequence at least 90% identical thereto.

37. The nucleic acid molecule of any one of claims 1-35, wherein the AAV expression cassette comprises a nucleic acid sequence SEQ ID NO: 11, or a sequence at least 90% identical thereto.

38. A plasmid, comprising the nucleic acid molecule of any one of claims 1-37.

39. A cell, comprising the nucleic acid molecule of any one of claims 1-37 or the plasmid of claim 38.

40. A method of producing a recombinant AAV vector, the method comprising contacting an AAV producer cell with the nucleic acid molecule of any one of claims 1-37 or the plasmid of claim 38.

41. A recombinant AAV vector produced by the method of claim 40.

42. The recombinant AAV vector of claim 41, wherein the vector is of a serotype selected from AAV1, AAV2, AAV3, AAV4, AAV5, AAV6, AAV7, AAV8, AAV10, AAV11, AAV12, AAVrh8, AAVrh10, AAVrh32.33, AAVrh74, Avian AAV and Bovine AAV.

43. The recombinant AAV vector of claim 41 or claim 42, wherein the recombinant AAV vector is a single-stranded AAV (ssAAV).

44. The recombinant AAV vector of claim 41 or claim 42, wherein the recombinant AAV vector is a self-complementary AAV (scAAV).

45. The recombinant AAV vector of claim 41, wherein the AAV vector comprises a capsid protein of AAV1, AAV2, AAV3, AAV4, AAV5, AAV6, AAV7, AAV8, AAV9, AAV10, AAV11, AAV12, AAVrh8, AAVrh10, AAVrh32.33, AAVrh74, Avian AAV or Bovine AAV.

46. The recombinant AAV vector of claim 41, wherein the AAV vector comprises a capsid protein with one or more substitutions or mutations, as compared to a wild type AAV capsid protein.

47. The recombinant AAV vector of claim 41, wherein the AAV vector comprises a capsid protein comprising:
a. (i) the amino acid sequence of SEQ ID NO: 15, or a sequence at least 90% identical thereto, or
b. (ii) the amino acid sequence of SEQ ID NO: 16, or a sequence at least 90% identical thereto, or
c. (iii) the amino acid sequence of SEQ ID NO: 17, or a sequence at least 90% identical thereto.

48. The recombinant AAV vector of claim 47, wherein the AAV vector comprises a capsid protein comprising the amino acid sequence of SEQ ID NO: 15, or a sequence at least 90% identical thereto.

49. The recombinant AAV vector of claim 48, wherein the AAV vector comprises a capsid protein comprising the amino acid sequence of SEQ ID NO: 15.

50. The recombinant AAV vector of claim 47, wherein the AAV vector comprises a capsid protein comprising the amino acid sequence of SEQ ID NO: 16, or a sequence at least 90% identical thereto.

51. The recombinant AAV vector of claim 50, wherein the AAV vector comprises a capsid protein comprising the amino acid sequence of SEQ ID NO: 16.

52. The recombinant AAV vector of claim 47, wherein the AAV vector comprises a capsid protein comprising the amino acid sequence of SEQ ID NO: 17, or a sequence at least 90% identical thereto.

53. The recombinant AAV vector of claim 52, wherein the AAV vector comprises a capsid protein comprising the amino acid sequence of SEQ ID NO: 17.

54. A composition, comprising: (a) the nucleic acid molecule of any one of claims 1-37, the plasmid of claim 38, the cell of claim 39, or the recombinant AAV vector of any one of claims 41-53; and (b) a pharmaceutically acceptable carrier.

55. A method of expressing an Angelman syndrome-associated transgene in a tissue, comprising: contacting the tissue with the nucleic acid molecule of any one of claims 1-37, the plasmid of claim 38, the recombinant AAV vector of any one of claims 41-53, or the composition of claim 54, thereby expressing the Angelman syndrome-associated transgene in the tissue.

56. The method of claim 55, wherein the tissue comprises brain tissue.

57. The method of claim 55 or claim 56, wherein the tissue comprises neuronal cells.

58. The method of any one of claims 55-57, wherein the contacting step is performed in vitro, ex vivo, or in vivo.

59. The method of claim 58, wherein the contacting step is performed in vivo in a subject in need thereof.

60. The method of claim 59, wherein the contacting step comprises administering a therapeutically effective amount of the nucleic acid molecule, the plasmid, the recombinant AAV vector, or the composition to the subject.

61. The method of claim 59 or claim 60, wherein the subject suffers from, or is at a risk of developing, the Angelman syndrome.

62. A method for treating Angelman syndrome in a subject in need thereof, comprising administering to the subject a therapeutically effective amount of the nucleic acid molecule of any one of claims 1-37, the plasmid of claim 38, the cell of claim 39, the recombinant AAV vector of any one of claims 41-53, or the composition of claim 54, thereby treating Angelman syndrome in the subject.

63. The method of claim 62, wherein the subject suffers from, or is at a risk of developing, the Angelman syndrome.

64. The method of any one of claims 61-63, wherein the Angelman syndrome is associated with, promoted by, or caused by a genetic mutation.

65. The method of claim 64, wherein the genetic mutation comprises a mutation in the human UBE3A gene.

66. The method of claim 64, wherein the genetic mutation comprises a mutation in the chromosomal region 15q11-q13.

67. The method of any one of claims 61-66, wherein the method comprises diminishing the severity of; delaying the onset or progression of; and/or eliminating a symptom of the Angelman syndrome.

68. The method of claim 67, wherein the symptom of the Angelman syndrome comprises: (a) developmental delay, (b) intellectual disability, (c) speech impairment, (d) gait ataxia, (e) tremulousness of the limbs, (f) frequent laughing or smiling, (g) excitability, (h) microcephaly, (i) seizures, (j) trouble sleeping, (k) tongue thrusting, (l) hand flapping, (m) curved spine or (n) any combination thereof.

69. The method of any one of claims 61-68, wherein the method comprises prolonging the survival of the subject, as compared to a control subject having Angelman syndrome, wherein the control subject has not been administered the therapeutically effective amount, or as compared to the expected survival of the subject prior to administration of the therapeutically effective amount.

70. The method of any one of claim 60-69, wherein the subject is a human subject.
